(19) United States
(12) Patent Application Publication
(10) Pub. No.: US 2015/0045367 A1
(43) Pub. Date: Feb. 12, 2015

(54) CINNAMIC ACID HYDROXYAMIDES AS INHIBITORS OF HISTONE DEACETYLASE 8

(71) Applicant: PHARMACYCLICS, INC., Sunnyvale, CA (US)

(72) Inventors: Erik Verner, Belmont, CA (US); Wei Chen, Fremont, CA (US); Sriram Balasubramanian, San Carlos, CA (US)

(21) Appl. No.: 14/368,475

(22) PCT Filed: Dec. 19, 2012
(86) PCT No.: PCT/US2012/070671

§ 371 Date: Jun. 24, 2014

Related U.S. Application Data
(60) Provisional application No. 61/581,459, filed on Dec. 29, 2011.

(51) Int. Cl. C07D 405/06; C07D 213/89; C07D 211/46; C07D 295/088; C07D 211/42; C07D 213/82; C07C 235/38; C07C 311/08; C07C 235/34; C07D 401/06

(52) U.S. Cl. CPC C07D 405/06; C07C 235/34; C07D 213/89; C07D 211/46; C07D 401/06; C07D 211/42; C07D 213/82; C07C 235/38; C07C 311/08; C07D 295/088
USPC 514/239.2; 564/161; 514/622; 546/300; 514/351; 546/242; 514/315; 546/194; 514/318; 546/214; 514/326; 546/316; 514/355; 564/155; 514/616; 564/162; 514/618; 544/174

(57) ABSTRACT

Described herein are compounds and pharmaceutical compositions containing such compounds, which inhibit the activity of histone deacetylase 8 (HDAC8). Also described herein are methods of using such HDAC8 inhibitors, alone and in combination with other compounds, for treating diseases or conditions that would benefit from inhibition of HDAC8 activity.

### RELATED APPLICATIONS

This application claims the benefit of U.S. provisional patent application No. 61/581,459 entitled “CINNAMIC ACID HYDROXYAMIDES AS INHIBITORS OF HISTONE DEACETYLASE 8” filed on Dec. 29, 2011, which is incorporated by reference in its entirety.

### FIELD OF THE INVENTION

Described herein are compounds, methods of making such compounds, pharmaceutical compositions and medicaments that include such compounds, and methods of using such compounds to inhibit the activity of histone deacetylase 8.

### BACKGROUND OF THE INVENTION

Histone deacetylases (HDACs) catalyze the removal of acetyl groups from histones, proteins that organize and modulate the structure of chromatin in nucleosomes. HDAC-mediated deacetylation of chromatin-bound histones regulates the expression of a variety of genes throughout the genome. Importantly, HDACs have been linked to cancer, as well as other health conditions. To date, eleven major HDAC isoforms have been described (HDACs 1-11). HDACs are categorized into two classes. Class I HDACs include HDAC1, HDAC2, HDAC3, HDAC8 and HDAC11. Class II HDACs include HDAC4, HDAC5, HDAC6, HDAC7, HDAC9 and HDAC10. Small molecule HDAC inhibitors that are isoform-selective are useful as therapeutic agents with reduced toxicity and as tools for probing the biology of the HDAC isoforms.

### SUMMARY OF THE INVENTION

In one aspect provided herein are substituted cinnamic acid hydroxyamide compounds and other HDAC8 inhibitors, pharmaceutically acceptable salts, pharmaceutically acceptable N-oxides, pharmaceutically active metabolites, pharmaceutically acceptable prodrugs, and pharmaceutically acceptable solvates thereof. In some embodiments, the compounds described herein inhibit HDAC8 activity. In some embodiments, the compounds described herein are used to treat mammals where inhibition of HDAC8 activity provides benefit. Compounds described herein are HDAC8 inhibitors.

In one aspect, described herein is a compound having a structure of Formula (I):

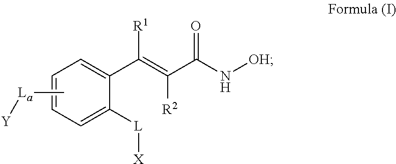

wherein:
R¹ and R² are each independently H, OH, halogen, or C₁-C₆alkyl;
L and Lₐ are each independently a bond, O, S, NR³, —NR¹⁰C(═O)—R¹¹, S(═O), S(═O)₂, NHS(═O)₂, —C₁-C₆alkylene-, —C₂-C₆alkenylene-, —C₂-C₆alkynylene-, —C₁-C₆heteroalkylene-, —C₁-C₆alkylene-O—, —C₁-C₃alkylene-O—C₁-C₃alkylene-, —C₁-C₆alkylene-NR³—, —C₁-C₃alkylene-NR³—C₁-C₃alkylene-, —C₁-C₆alkylene-C(═O)NR³—, —C₁-C₃alkylene-C(═O)NR³—C₁-C₃alkylene-, —C₁-C₆alkylene-NR³C(═O)—, —C₁-C₃alkylene-NR³C(═O)—C₁-C₃alkylene-, —C₁-C₆alkylene-S—, —C₁-C₃alkylene-S—C₁-C₃alkylene-, —C₁-C₆alkylene-S(═O)—, —C₁-C₃alkylene-S(═O)—C₁-C₃alkylene, —C₁-C₆alkylene-S(═O)₂—, —C₁-C₃alkylene-S(═O)₂—C₁-C₃alkylene, —C(═O)—, or —C(═O)—C₁-C₆alkylene;
X is a substituted or unsubstituted group selected from among aryl, heteroaryl, C₃-C₁₀cycloalkyl, and C₂-C₁₀heterocycloalkyl; where if X is substituted, then X is substituted with 1, 2, 3, 4, or 5 groups selected from among halogen, C₁-C₆alkoxy, C₁-C₆-fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹)₂, —S(═O)₂N(R¹)₂, —NR—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
Y is H or a substituted or unsubstituted group selected from among C₁-C₆alkyl, —CO₂R¹⁰, —C(═O)R¹¹, —NR¹⁰C(═O)—R¹¹, —C(═O)N(R¹)₂, aryl, heteroaryl, C₃-C₁₀cycloalkyl, and C₂-C₁₀heterocycloalkyl; where if Y is substituted, then Y is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
   R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
   R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is H, C₁-C₆alkyl, phenyl or benzyl;

or a pharmaceutically acceptable salt, pharmaceutically acceptable N-oxide, or pharmaceutically acceptable prodrug thereof.

For any and all of the embodiments, substituents are selected from among from a subset of the listed alternatives. For example, in some embodiments, R¹ is hydrogen or C₁-C₆alkyl. In some other embodiments, R² is hydrogen or C₁-C₆alkyl. In other embodiments, R¹ and R² are each hydrogen.

In one embodiment, L is O or S.

In another embodiment, X is a substituted or unsubstituted aryl. In yet another embodiment, aryl is phenyl.

In a further embodiment, phenyl is substituted with at least one Cl, Br, I, or F. In yet a further embodiment, phenyl is substituted with at least two of Cl, Br, I, or F.

In one embodiment, phenyl is substituted with at least one C₁-C₆alkyl. In another embodiment, C₁-C₆alkyl is methyl, ethyl, n-propyl, iso-propyl, n-butyl, iso-butyl, or tert-butyl.

In yet another embodiment, C₁-C₆alkyl is methyl.

In a further embodiment, phenyl is substituted with at least one C₁-C₆alkoxy. In yet a further embodiment, C₁-C₆alkoxy is selected from methoxy or ethoxy.

In one embodiment, X is a substituted or unsubstituted heteroaryl. In another embodiment, heteroaryl is pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, 4-azaindolyl, 5-azaindolyl, 6-azaindolyl, 7-azaindolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, imidazo[1,2-a]pyridinyl, thiophenopyridinyl, and furopyridinyl.

In yet another embodiment, heteroaryl is pyridinyl.

In a further embodiment, X is a substituted or unsubstituted C₃-C₁₀cycloalkyl. In yet a further embodiment, C₃-C₁₀cycloalkyl is cyclopropyl, cyclobutyl, cyclopentyl and cyclohexyl.

In one embodiment, X is a substituted or unsubstituted C₂-C₁₀heterocycloalkyl.

In another embodiment, C₂-C₁₀heterocycloalkyl is quinolizinyl, dioxinyl, piperidinyl, morpholinyl, thiomorpholinyl, thiazinyl, tetrahydropyridinyl, piperazinyl, oxazinanonyl, dihydropyrrolyl, dihydroimidazolyl, tetrahydrofuranyl, tetrahydropyranyl, dihydrooxazolyl, oxiranyl, pyrrolidinyl, pyrazolidinyl, dihydrothienyl, imidazolidinonyl, pyrrolidinonyl, dihydrofuranonyl, dioxolanonyl, thiazolidinyl, piperidinonyl, indolinyl, tetrahydroquinolinyl, tetrahydroisoquinolinyl, and tetrahydrothienyl. In yet another embodiment, C₂-C₁₀heterocycloalkyl is piperidinyl. In a further embodiment, piperidinyl is substituted with —CO₂R¹⁰, —C(═O)R¹¹ or —C(═O)N(R¹⁰)₂. In yet a further embodiment, piperidinyl is substituted with C(═O)R¹¹.

In one embodiment, R¹¹ is a substituted or unsubstituted C₁-C₆alkyl. In another embodiment, C₁-C₆alkyl is methyl, ethyl, n-propyl, iso-propyl, n-butyl, iso-butyl or tert-butyl.

In yet another embodiment, C₁-C₆alkyl is methyl or iso-propyl.

In a further embodiment, R¹¹ is a substituted or unsubstituted aryl. In yet a further embodiment, aryl is a phenyl group.

In one embodiment, R¹¹ is a substituted or unsubstituted heteroaryl. In another embodiment, heteroaryl is pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, 4-azaindolyl, 5-azaindolyl, 6-azaindolyl, 7-azaindolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, imidazo[1,2-a]pyridinyl, thiophenopyridinyl, and furopyridinyl. In yet another embodiment, heteroaryl is pyridinyl or furanyl.

In one aspect is a compound selected from (E)-N-hydroxy-3-(2-(3-methoxyphenoxy)phenyl)acrylamide; (E)-3-(2-(3-fluorophenoxy)phenyl)-N-hydroxyacrylamide; (E)-N-hydroxy-3-(2-(pyridin-3-yloxy)phenyl)acrylamide; (E)-N-hydroxy-3-(2-(pyridin-4-yloxy)phenyl)acrylamide; (E)-N-hydroxy-3-(2-(4-methoxyphenoxy)phenyl)acrylamide; (E)-3-(2-(3-chlorophenoxy)phenyl)-N-hydroxyacrylamide; (E)-3-(2-(3,4-dichlorophenoxy)phenyl)-N-hydroxyacrylamide; (E)-N-hydroxy-3-(2-(m-tolyloxy)phenyl)acrylamide; (E)-N-hydroxy-3-(2-phenoxyphenyl)acrylamide; (E)-N-hydroxy-3-(2-(p-tolyloxy)phenyl)acrylamide; (E)-3-(2-(4-chlorophenoxy)phenyl)-N-hydroxyacrylamide; (E)-3-(2-(4-fluorophenoxy)phenyl)-N-hydroxyacrylamide; (S,E)-3-(2-(1-benzoylpiperidin-3-yloxy)phenyl)-N-hydroxyacrylamide; (S,E)-3-(2-(1-acetylpiperidin-3-yloxy)phenyl)-N-hydroxyacrylamide; (S,E)-N-hydroxy-3-(2-(1-isobutyrylpiperidin-3-yloxy)phenyl)acrylamide; (E)-3-(2-(1-(furan-2-carbonyl)piperidin-4-yloxy)phenyl)-N-hydroxyacrylamide; (E)-3-(2-(1-acetylpiperidin-4-yloxy)phenyl)-N-hydroxyacrylamide; (E)-N-hydroxy-3-(2-(1-isobutyrylpiperidin-4-yloxy)phenyl)acrylamide; (E)-3-(2-(1-benzoylpiperidin-4-yloxy)phenyl)-N-hydroxyacrylamide; (E)-N-hydroxy-3-(2-(1-nicotinoylpiperidin-4-yloxy)phenyl)acrylamide; (R,E)-3-(2-(1-acetylpiperidin-3-yloxy)phenyl)-N-hydroxyacrylamide; (S,E)-3-(2-(1-(furan-2-carbonyl)piperidin-3-yloxy)phenyl)-N-hydroxyacrylamide; (R,E)-3-(2-(1-(furan-2-carbonyl)piperidin-3-yloxy)phenyl)-N-hydroxyacrylamide; (R,E)-N-hydroxy-3-(2-(1-isobutyrylpiperidin-3-yloxy)phenyl)acrylamide; (R,E)-3-(2-(1-benzoylpiperidin-3-yloxy)phenyl)-N-hydroxyacrylamide; (R,E)-N-hydroxy-3-(2-(1-nicotinoylpiperidin-3-yloxy)phenyl)acrylamide; (S,E)-N-hydroxy-3-(2-(1-nicotinoylpiperidin-3-yloxy)phenyl)acrylamide; (E)-N-(4-(4-fluorophenoxy)-3-(3-(hydroxyamino)-3-oxoprop-1-enyl)phenyl)nicotinamide; (E)-3-(5-acetamido-2-(4-fluorophenoxy)phenyl)-N-hydroxyacrylamide; (E)-3-(5-acetamido-2-(3-chlorophenoxy)phenyl)-N-hydroxyacrylamide; (E) —N-(4-(3-chlorophenoxy)-3-(3-(hydroxyamino)-3-oxoprop-1-enyl)phenyl)nicotinamide; (E)-N-(4-(3-fluorophenoxy)-3-(3-(hydroxyamino)-3-oxoprop-1-enyl)phenyl)nicotinamide; (E)-N-(4-(3,4-dichlorophenoxy)-3-(3-(hydroxyamino)-3-oxoprop-1-enyl)phenyl)nicotinamide; (E)-3-(5-acetamido-2-(3-fluorophenoxy)phenyl)-N-hydroxyacrylamide; (E)-3-(2-(4-fluorophenoxy)-5-(methylsulfonamido)phenyl)-N-hydroxyacrylamide; (E)-3-(2-(3-chlorophenoxy)-5-(methylsulfonamido)phenyl)-N-hydroxyacrylamide; (E)-3-(2-(4-fluorophenoxy)-5-(pyridin-3-ylmethylamino)phenyl)-N-hydroxyacrylamide; (E)-3-(2-(3-chlorophenoxy)-5-(pyridin-3-ylmethylamino)phenyl)-N-hydroxyacrylamide; (E)-3-(2-(3-chlorophenoxy)-5-(pyridin-2-ylmethylamino)phenyl)-N-hydroxyacrylamide; (E)-3-(2-(4-fluorophenoxy)-5-(pyridin-2-ylmethylamino)phenyl)-N-hydroxyacrylamide; (E)-3-(2-(3-fluorophenoxy)-5-(pyridin-3-ylmethylamino)phenyl)-N-hydroxyacrylamide; (E)-3-(2-(3,4-dichlorophenoxy)-5-(pyridin-3-ylmethylamino)phenyl)-N-hydroxyacrylamide; (E)-3-(2-(3-fluorophenoxy)-5-(pyridin-2-ylmethylamino)phenyl)-N-hydroxyacrylamide; (E)-3-(2-(3,4-dichlorophenoxy)-5-(pyridin-2-ylmethylamino)phenyl)-N-hydroxyacrylamide; (E)-3-(2-(3-chlorophenoxy)-4-(pyridin-3-ylmethoxy)phenyl)-N-hydroxyacrylamide; (E)-3-(2-(3-chlorophenoxy)-4-(2-morpholinoethoxy)phenyl)-N-hydroxyacrylamide; (E)-3-(2-(3-chlorophenoxy)-4-(2-(4-methylpiperazin-1-ypethoxy)phenyl)-N-hydroxyacrylamide; (E)-3-(2-(3-chlorophenoxy)-4-(2-(dimethylamino)ethoxy)phenyl)-N-hydroxyacrylamide; (E)-3-(2-(3-chlorophenoxy)-4-(2-methoxyethoxy)phenyl)-N-hydroxyacrylamide; (E)-3-(2-(3-fluorophenoxy)-4-(2-methoxyethoxy)phenyl)-N-hydroxyacrylamide; (E)-3-(4-(2-acetamidoethoxy)-2-(3-chlorophenoxy)phenyl)-N-hydroxyacrylamide; (E)-3-(2-(3-fluorophenoxy)-4-(2-(dimethylamino)ethoxy)phenyl)-N-hydroxyacrylamide; (E)-3-(2-(3-fluorophenoxy)-4-(2-(4-methylpiperazin-1-ypethoxy)phenyl)-N-hydroxyacrylamide; (E)-3-(2-(4-fluorophenoxy)-4-(2-methoxyethoxy)phenyl)-N-hydroxyacrylamide; (E)-3-(2-(4-fluorophenoxy)-4-(pyridin-3-ylmethoxy)phenyl)-N-hydroxyacrylamide; (E)-3-(2-(4-fluorophenoxy)-4-(2-(dimethylamino)ethoxy)phenyl)-N-hydroxyacrylamide; (E)-3-(4-(2-acetamidoethoxy)-2-(4-fluorophenoxy)phenyl)-N-hydroxyacrylamide; (E)-3-(2-(4-fluorophenoxy)-4-(2-(4-methylpiperazin-1-ypethoxy)phenyl)-N-hydroxyacrylamide; (E)-3-(2-(3,4-dichlorophenoxy)-4-(2-methoxyethoxy)phenyl)-N-hydroxyacrylamide; (E)-3-(4-(2-acetamidoethoxy)-2-(3,4-dichlorophenoxy)phenyl)-N-hydroxyacrylamide; (E)-3-(2-(3,4-dichlorophenoxy)-4-(2-morpholinoethoxy)phenyl)-N-hydroxyacrylamide; (E)-3-(2-(3,4-dichlorophenoxy)-4-(pyridin-3-ylmethoxy)phenyl)-N-hydroxyacrylamide; an active metabolite, pharmaceutically acceptable solvate, pharmaceutically acceptable salt, pharmaceutically acceptable N-oxide, or pharmaceutically acceptable prodrug thereof.

Further disclosed herein are pharmaceutical compositions comprising a compound of Formula (I), (II), (III), (IIIa), (IV), (IVa), (IVb), or (IVc) or an active metabolite, pharmaceutically acceptable solvate, pharmaceutically acceptable salt, pharmaceutically acceptable N-oxide, or pharmaceutically acceptable prodrug thereof and a pharmaceutically acceptable diluent, excipient, or carrier. In some embodiments, the pharmaceutical compositions are formulated for intravenous injection, subcutaneous injection, oral administration, inhalation, nasal administration, topical administration, ophthalmic administration or otic administration. In some embodiments, the pharmaceutical compositions are formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

Additionally disclosed herein are pharmaceutical compositions comprising a HDAC8 inhibitor compound described herein, or a pharmaceutically acceptable salt, pharmaceutically acceptable N-oxide, or pharmaceutically acceptable prodrug thereof and a pharmaceutically acceptable diluent, excipient, or carrier. In some emvodiments, the pharmaceutical compositions are formulated for intravenous injection, subcutaneous injection, oral administration, inhalation, nasal administration, topical administration, ophthalmic administration or otic administration. In some embodiments, the pharmaceutical compositions are formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

Disclosed herein, in certain embodimens, are methods of treating T-cell lymphoma or leukemia in a mammal comprising administering a HDAC8 8 inhibitor compound described herein. In one aspect, the mammal is a human. In some embodiments, compounds described herein are orally administered.

In one aspect is the use of a HDAC8 8 inhibitor compound described herein for treating T-cell lymphoma or leukemia in a mammal. In one aspect, the mammal is a human. In some embodiments, compounds described herein are orally administered.

In one aspect is the use of a HDAC8 8 inhibitor compound described herein in the manufacture of a medicament for treating T-cell lymphoma or leukemia in a mammal. In one aspect, the mammal is a human. In some embodiments, compounds described herein are orally administered.

Also described herein are methods of treating a disease or condition mediated by interleukin-1 beta (IL-1b) or IL-18 in a mammal in need thereof, comprising administering to the mammal a therapeutically effective amount of a HDAC8 8 inhibitor compound described herein, or a pharmaceutically acceptable salt, pharmaceutically acceptable N-oxide, pharmaceutically active metabolite, pharmaceutically acceptable prodrug, or pharmaceutically acceptable solvate thereof. In one aspect, the disease or condition is selected from among osteoarthritis, rheumatoid arthritis, septic arthritis, gout, pseudogout, juvenile arthritis, Still's disease, Ankylosing spondylitis, systemic lupus erythematosus (SLE), Henoch-Schnlein purpura, psoriatic arthritis, reactive arthritis (Reiter's syndrome), hemochromatosis, hepatitis, Wegener's granulomatosis, Familial Mediterranean fever (FMF), HIDS (hyperimmunoglobulinemia D and periodic fever syndrome), TRAPS (TNF-alpha receptor associated periodic fever syndrome), inflammatory bowel disease, Crohn's Disease, ulcerative colitis, recurrent fever, anemia, leukocytosis, asthma, chronic obstructive pulmonary disease, and myalgia. In one aspect, the method further comprises administering to the mammal a second therapeutic agent, selected from among tacrolimus, cyclosporin, rapamicin, methotrexate, cyclophosphamide, azathioprine, mercaptopurine, mycophenolate, or FTY720, prednisone, cortisone acetate, prednisolone, methylprednisolone, dexamethasone, betamethasone, triamcinolone, beclometasone, fludrocortisone acetate, deoxycorticosterone acetate, aldosterone, aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502, JTE-522, L-745,337 and NS398, leflunomide, gold thioglucose, gold thiomalate, aurofin, sulfasalazine, hydroxychloroquinine, minocycline, infliximab, etanercept, adalimumab, abatacept, anakinra, interferon-β, interferon-γ, interleukin-2, allergy vaccines, antihistamines, antileukotrienes, beta-agonists, theophylline, and anticholinergics. In one aspect, the mammal is a human. In some embodiments, compounds described herein are orally administered.

In one aspect, HDAC8 8 inhibitor compounds described herein are for use in treating a disease or condition mediated by interleukin-1 beta (IL-1b) or IL-18 in a mammal. In one aspect, the disease or condition is selected from among osteoarthritis, rheumatoid arthritis, septic arthritis, gout, pseudogout, juvenile arthritis, Still's disease, Ankylosing spondylitis, systemic lupus erythematosus (SLE), Henoch-Schnlein purpura, psoriatic arthritis, reactive arthritis (Reiter's syndrome), hemochromatosis, hepatitis, Wegener's granulomatosis, Familial Mediterranean fever (FMF), HIDS (hyperimmunoglobulinemia D and periodic fever syndrome), TRAPS (TNF-alpha receptor associated periodic fever syndrome), inflammatory bowel disease, Crohn's Disease, ulcerative colitis, recurrent fever, anemia, leukocytosis, asthma, chronic obstructive pulmonary disease, and myalgia. In a further aspect, the HDAC8 8 inhibitor compounds described herein are used in combination with a second therapeutic agent, selected from among tacrolimus, cyclosporin, rapamicin, methotrexate, cyclophosphamide, azathioprine, mercaptopurine, mycophenolate, or FTY720, prednisone, cortisone acetate, prednisolone, methylprednisolone, dexamethasone, betamethasone, triamcinolone, beclometasone, fludrocortisone acetate, deoxycorticosterone acetate, aldosterone, aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502, JTE-522, L-745,337 and NS398, leflunomide, gold thioglucose, gold thiomalate, aurofin, sulfasalazine, hydroxychloroquinine, minocycline, infliximab, etanercept, adalimumab, abatacept, anakinra, interferon-β, interferon-γ, interleukin-2, allergy vaccines, antihistamines, antileukotrienes, beta-agonists, theophylline, and anticholinergics. In one aspect, the mammal is a human. In some embodiments, compounds described herein are orally administered.

In one aspect is the use of a HDAC8 8 inhibitor compounds described herein in the manufacture of a medicament for treating a disease or condition mediated by interleukin-1 beta (IL-1b) or IL-18 in a mammal. In one aspect, the mammal is a human. In some embodiments, compounds described herein are orally administered.

In any of the aforementioned embodiments involving the treatment with a HDAC8 inhibitor compound are further embodiments comprising administering at least one additional agent in addition to the administration of a HDAC8 inhibitor compound. Each agent is administered in any order, including simultaneously.

In some embodiments, compounds described herein are used for inhibiting the activity of HDAC8 or for the treatment of a disease or condition that would benefit from inhibition of the activity of HDAC8.

In some embodiments, compounds described herein are used for the formulation of a medicament for the inhibition of HDAC8 activity.

Articles of manufacture, which include packaging material, a HDAC8 inhibitor compound described herein, within the packaging material, and a label that indicates that the compound or composition, or pharmaceutically acceptable salt, pharmaceutically acceptable N-oxide, prodrug, or pharmaceutically acceptable solvate thereof, is used for inhibiting the activity of HDAC8, or for the treatment, prevention or amelioration of one or more symptoms of a disease or condition that would benefit from inhibition of the activity of HDAC8, are provided.

Other objects, features and advantages of the methods, compounds, and compositions described herein will become apparent from the following detailed description. It should be understood, however, that the detailed description and the specific examples, while indicating specific embodiments, are given by way of illustration only, since various changes and modifications within the spirit and scope of the disclosure will become apparent from this detailed description.

### DETAILED DESCRIPTION

Covalent modification of histone proteins through acetylation and deacetylation is an important determinant of chromatin structure and a regulator of gene expression. Acetylation of histone proteins occurs on lysine residues near the N-termini of these proteins. In conjunction with other modifications of histone proteins and DNA, the acetylation state of histones determines whether the chromatin is in a condensed, transcriptionally silent state or in a form more accessible to the transcription machinery of the cell. In general, hyperacetylation of histone proteins is associated with transcriptional activation of genes. The steady-state histone acetylation level arises from the opposing action of histone acetyltransferase (HAT) and histone deacetylase (HDAC) enzymes.

Histone deacetylases (HDACs) catalyze the removal of acetyl groups from lysine ε-amino groups near the N-termini of histones. This reaction promotes the condensation of chromatin, leading to repression of transcription.

HDAC inhibitors (HDIs) modify gene expression positively or negatively in a cell- and gene-specific manner. HDIs increase the accumulation of acetylated histones, directly influencing chromatin structure and, thereby, the relationship of the nucleosome to gene promoter elements.

Histone deacetylase (HDAC) enzymes modulate gene expression through the deacetylation of acetylated lysine residues on histone proteins. They operate in biological systems as part of multiprotein corepressor complexes. Histone deacetylases have been grouped into three classes. Class I and class II histone deacetylases (HDACs) are zinc containing hydrolase enzymes. The division of the proteins into classes I and II is based on protein size, sequence similarity, and organization of the protein domains.

Members of class I are related to the yeast RPD3 gene product. Class I HDACs include: HDAC1; HDAC2; HDAC3; HDAC8; HDAC11.

HDAC8 is a 377 residue, 42 kDa protein localized to the nucleus of a wide array of tissues, as well as several human tumor cell lines. The wild-type form of full length HDAC8 is described in GenBank Accession Number NP 060956; Buggy, J. J. et al., Biochem. J., 350 (Pt 1), 199-205 (2000). The HDAC8 structure was solved with four different hydroxamate inhibitors bound (Somoza et al., Structure, 2004, 12, 1325).

Class II are homologues of the yeast HDA1 protein, and include: HDAC4; HDAC5; HDAC6; HDAC7; HDAC9; HDAC O.

Class II HDACs have been further subdivided into classes IIa (HDACs 4, 5, 7, and 9) and IIb (HDACs 6 and 10).

The third class of deacetylases consists of the members of the Sir2 family of enzymes. These enzymes have histone deacetylase activity but are structurally and evolutionarily unrelated to the class I and class II proteins. They are (nicotinamide adenine dinucleotide) NAD-dependent and unlike class I HDACs and class II HDACs, they do not contain a catalytic zinc site.

In the cell, HDAC proteins are recruited as part of multicomponent repressor complexes. Several HDAC containing complexes have been characterized, including the N-CoR/SMRT, Sin3, NuRD, and CoREST complexes. Within these complexes, HDACs 1 and 2 typically interact with the mSin3, Mi-2, or CoREST proteins. HDAC3 and the class IIa HDACs have been shown to interact with SMRT and the related N-CoR protein. A large number of transcription factors have been shown to bind to one of the corepressor complexes as a means of regulating transcription. The recruitment of HDACs by DNA-binding proteins allows histone deacetylation to be directed toward specific regions of the chromatin in order to promote targeted transcriptional repression.

HDAC proteins are promising therapeutic targets on account of their involvement in regulating genes involved in cell cycle progression and control. Inhibition of HDACs has been shown to upregulate genes, including p21WAF/CIP1, p2′7, p53, and cyclin E, and to downregulate genes such as cyclin A and cyclin D. Growth inhibition in several lines of cancer cells has been observed upon treatment with HDAC inhibitors, and in vivo studies have shown that some of these inhibitors are efficacious in slowing tumor growth. The biological activity of each of the HDAC isozymes is determined by a combination of the intrinsic activity of the enzyme and the effects of cofactor binding on reactivity and substrate recognition (Schultz et al., Biochemistry, 2004, 43, 11083-11091).

Non-selective HDAC inhibitors inhibit the deacetylase activity of most, if not all, of the HDACs with equal potency. The mechanisms of the anticancer effects of SAHA, a non-selective HDAC inhibitor, are not completely understood, and likely result from both altered gene expression and altered function of proteins regulating cell proliferation and cell death pathways. Non-selective HDAC inhibitors, such as SAHA, induce the accumulation of acetylated histone proteins and non histone proteins. Non-histone proteins that are acetylated include, but are not limited to:

Bcl-6 (Oncoprotein); LEF/TCF (Lymphoid enhancer factor); P53 (Tumor suppressor); Ku70 (Autoantigen with multiple function, including DNA repair); H₁F-1a (angiogenesis); GATA-1 (Transcription factor); WRN (Werner helicase); E2F-1 (Transcription factor); Smad7 (Transcription factor); Rb (Tumor suppressor); TFIIF (Transcription machinery); c-Jun (Transcription factor); α-Tubulin (Structural protein); HMGI(Y) (Chromatin structure); ACTR (Nuclear receptor coactivator); Androgen Receptor (Signal transduction); EKLF (Erythroid kruppel-like factor); YY-1 (Transcription factor); NF-κB(ReIA) (Transcription factor); MyoD (Transcription factor); Importin a7 (Nuclear pore protein); Hsp90 (Chaperone protein); TFIIE (Transcription machinery); b-Catenin (Signaltransduction); TFJB (Transcription factor).

Genes whose transcription is altered by histone deacetylase inhibitors include:

1) Genes that are induced by HDAC inhibitors: Cell cycle (pl and cyclin E); Proapoptotic (Bak, BAX, CD95, and its ligand gelsolin, GADD45(3, p53, Apaf-1 DFF45a, Bim, BAD, TRAIL, DRS, Fas and its ligand, and Caspase 9, -8 and -3); Redox Components (Thioredoxin-binding protein-1, thioredoxin, glutaredoxin and methallothionein 1L); Chromatin structure (Histone H₂B); Retinoic acid pathway (RARβ).

2) Genes that are repressed by HDAC inhibitors: Cell cycle (Cyclin D1 and A, and thymidylate synthase); Antiapoptotic (Bc1-2, Bc1-XL, c-FLIP, survivin, XIAP); Angiogenic factor (Vascular endothelial growth factor and HIF-Loc); Lipopolysaccharide-induced inflammatory cytokines (TNF-a, IFN-g and IL-1b and -6); Signaltransducer and activator of transcription 5-controlled genes (STATS).

HDAC enzymes or isoforms appear to be involved in many different types of cancer. Inhibition of HDACs with HDAC inhibitors results in multiple and desirable anti-cancer effects such as, but not limited to, (i) the inhibition of cancer cell proliferation, (ii) the induction of apoptosis (cell death) of cancer cells, (iii) cell cycle regulation, (iv) the induction of tumour suppressor genes, and (v) the blocking of tumour angiogenesis (development of new tumour blood vessels). These multiple effects provided by HDAC inhibitors provide a method of treating cancer.

Interest in histone deacetylase enzymes (HDACs) as targets for pharmaceutical development has centered on the role of HDACs in regulating genes associated with cell-cycle progression and the development and progression of cancer (Kramer et. al. Trends Endocrinol. Metab. 12, 294-300, (2001)). Several studies have shown that treatment of various cell lines with HDAC inhibitors leads to hyper acetylation of histone proteins and cell-cycle arrest in late G₁ phase or at the G₂/M transition. Genes involved in the cell cycle that have been shown to be up regulated by HDAC inhibitors include p21, p2′7, p53 and cyclin E. Cyclin A and cyclin D have been reported to be down regulated by HDAC inhibitors. In tumor cell lines, several studies have shown that treatment with HDAC inhibitors lead to growth inhibition, growth arrest, terminal differentiation and/or apoptosis. In vivo studies have demonstrated growth inhibition of tumors and a reduction in tumor metastasis as a result of treatment with HDAC inhibitors.

The clearest link between abnormal HDAC activity and cancer occurs in acute promyelocytic leukemia. In this condition, a chromosomal translocation leads to the fusion of the retinoic acid receptor RARa with the promyelocytic leukemia (PML) or promyelocytic leukemia zinc-finger (PLZF) proteins. Both PML-RARa and PLZF-RARa promote the progression of leukemia by repressing retinoic acid-regulated genes through the abnormal recruitment of SMRT-mSin3-HDAC complex (Lin et. al. Nature 391, 811-814 (1998)); Grignani et al. Nature 391, 815-818 (1998)). Whereas the PML-RARa form of the disease is treatable with retinoic acid, the PLZF-RARα form is resistant to this treatment. For a patient with the retinoic acid-resistant form of the disease, the addition of the HDAC inhibitor sodium butyrate to the dosing regimen led to complete clinical and cytogenic remission (Warrell et al. J. Natl. Cancer. Inst. 90, 1621-1625, (1998)). HDACs have also been associated with Huntington's disease (Steffan, et al., Nature 413:739-744, “Histone deacetylase inhibitors arrest polyglutamine-dependent neurodegeneration in Drosophila”).

In general, almost all of the inhibitors targeting HDACs are broad spectrum compounds, inhibiting all of the HDAC isoforms with equal potency. These broad spectrum HDAC inhibitors cause the induction of differentiation, growth arrest and/or apoptosis in a large number of tumor cell lines in vitro.

Clinical administration of broad spectrum HDAC inhibitors (pan HDAC inhibitors) has been associated with many dose limiting toxicities. These include thrombocytopenia, and other hematological toxicities, QTc prolongation and other cardiac toxicities, nausea, fever, fatigue, and anorexia (For example, see Clinical Cancer Research 2003, 9(10), 3578-3588; Clinical Cancer Research 2002, 8(7), 2142-2148; and Proceedings of the American Association of Cancer Research 2005, 46, Abs 3978). Selective HDAC inhibitors that selectively inhibit only one HDAC isoform, as opposed to a pan-selective inhibitor, is expected to produce a drug with an improved toxicity profile.

Adverse effects in humans have been reported in several clinical trials using pan-HDAC inhibitors. Originally designed for oncological applications, such toxicities might not be crucial when taking into consideration their therapeutic effects and the high mortality rate of cancer.

Described herein are HDAC8 inhibitor compounds. Compounds described herein selectively inhibit HDAC8 over other HDAC isoforms (e.g. HDACs 1, 2, 3, 6, 10, and 11).

As described herein, HDAC8 is expressed primarily in delta cells of the islets of Langerhans in the pancreas; in small intestinal epithelial cells; and in neuroendocrine cells. Of note, delta cells express and secrete somatostatin, a peptide hormone that inhibits the secretion of insulin and growth hormone. Without being bound by theory, it is believed that HDAC8 activity drives the expression of somatostatin in delta cells. Thus, inhibiting HDAC8 activitity is expected to decrease somatostatin expression and secretion from delta cells, and consequently increase systemic insulin and growth hormone levels.

Described herein are methods for inhibiting somatostatin expression in a subject by administering to the subject a selective HDAC8 inhibitor composition. Further, described herein are methods for treating a subject suffering from an insulin deficiency or a growth hormone deficiency by administering a selective HDAC8 inhibitor to the subject.

### T-cell Lymphomas or Leukemias

HDAC8 is expressed at unusually high levels in tumor cell lines, e.g., Jurkat, HuT78, K562, PC3, and OVCR-3. In fact, as described herein, inhibiting HDAC8 activity decreases proliferation of T-cell derived tumor cells (e.g., Jurkat cells) by apoptosis. In contrast, HDAC8 inhibition does not affect the proliferation of either non-cancerous cells (e.g., peripheral blood mononuclear cells) or tumor cell lines other than T-cell derived lines. Thus, selective HDAC8 inhibitors are useful for slowing or arresting the progression of T-cell derived cancers with lessened or no toxicity to non-cancerous cells.

Described herein are methods for treating a subject suffering from a T-cell lymphoma by administering to the subject a selective HDAC8 inhibitor composition. Also described herein are methods for treating a subject suffering from a T-cell lymphoma by administering to the subject a population of autologous T-cells that have been exposed to a selective HDAC8 inhibitor composition ex vivo.

In some embodiments, selective HDAC8 inhibitor compounds and compositions thereof are used to treat a subject suffering from a T-cell lymphoma, e.g., a peripheral T-cell lymphoma, a lymphoblastic lymphoma, a cutaneous T-cell lymphoma, or an adult T-cell lymphoma.

In some embodiments, the T-cell lymphoma treatment method includes administering to a subject a therapeutically effective amount of a selective HDAC8 inhibitor pharmaceutical composition.

In other embodiments, the T-cell lymphoma treatment includes administering, in addition to a selective HDAC8 inhibitor pharmaceutical composition, one or more additional anti-cancer agents described herein in any combination.

The methods described herein include administering a pharmaceutical composition containing a selective HDAC8 inhibitor in a quantity sufficient to decrease HDAC8 deacetylase activity in vivo by a therapeutically effective amount. In some embodiments, cells derived from a subject to be treated (i.e. autologous cells) are exposed, ex vivo, to a pharmaceutical composition containing a selective HDAC8 inhibitor composition in a quantity sufficient to decrease HDAC8 deacetylase activity in vitro.

In one embodiment, T-cells from a donor subject suffering a T-cell lymphoma are cultured and expanded, ex vivo, in the presence of a selective HDAC8 inhibitor at a concentration that is effective for selectively killing transformed T-cells. Afterwards, the expanded T-cell population, free of transformed T-cells, are introduced into the donor subject. T-cell culture, in vitro expansion, and in vivo transfer is described in, e.g., Porter et al. (2006), Blood, 107(4):1325-1331; Rapoport et al. (2005), Nat. Med., 1230-1237; Laport et al. (2003), Blood, 102(6):2004-2013.

### Cytokine-Modulated Health Conditions

In some embodiments, a subject is administered a therapeutically effective amount of a selective HDAC8 inhibitor to decrease secretion of one or more inflammatory cytokines (e.g., IL-113).

In some embodiments a selective HDAC8 inhibitor compound is administered to a subject to decrease the systemic levels of one or more inflammatory cytokines including, e.g., IL-1β, IL-6, IL-18, TNF-α, MCP-1, or MIP-1α.

As described herein, selective HDAC8 inhibitor compounds described herein reduce the secretion of proinflammatory cytokines including but not limited to interleukin-1 beta (IL-1β). Thus, HDAC8 is the HDAC enzyme involed in cytokine secretion. The use of selective HDAC8 inhibitor compounds provides a method of reducing cytokine secretion with reduced toxicity, due to the selective inhibition of one HDAC isoform (vs. the use of pan-HDAC inhibitors that inhibit all of the HDAC isoforms).

Selective HDAC8 inhibitor compounds described herein inhibit, in a dose dependent fashion, lipopolysaccharide (LPS) and/or ATP stimulated secretion of IL-1β from purified human peripheral blood mononuclear cells (PBMCs) as well as from the monocyte cell line THP-1. In some embodiments, the EC₅₀ for inhibition ranges from about 0.5 micromolar to about 5 micromolar.

The production and secretion of IL-1β is via a non-classical pathway of protein secretion, involving potassium efflux, the autocatalytic processing of procaspase-1, the cleavage by active caspase-1 of the IL-1β precursor, the influx of calcium ions, and the activation of specific phospholipases including PLA-2. In some embodiments, selective HDAC8 inhibitor compounds described herein inhibit one or more steps in this secretory pathway.

As described herein, selective HDAC8 inhibitors are used to treat diseases or conditions that are mediated or linked to IL-1β secretion and activity. In certain autoimmune diseases or conditions, IL-1β is contributes to the signs and symptoms of the diseases or conditions (for examples of such Burger et al., Best Practice & Research Clinical Rheumatology, Vol. 20, No. 5, pp. 879-896, 2006; Dayer et al., Current Opinions in Rheum., 2001, 13:170-176; Abramson et al., Rheumatology, 2002; 41; 972-980); selective HDAC8 inhibitor compounds are used to treat such diseases or conditions. As described herein, selective HDAC8 inhibitor compounds are used to inhibit IL-1β secretion and thus find utility in the treatment of diseases or conditions that are linked to IL-1β secretion and activity, which include, but are not limited to, osteoarthritis, rheumatoid arthritis, septic arthritis, gout, pseudogout, juvenile arthritis, Still's disease, Ankylosing spondylitis, systemic lupus erythematosus (SLE), Henoch-Schnlein purpura, psoriatic arthritis, reactive arthritis (Reiter's syndrome), hemochromatosis, hepatitis, Wegener's granulomatosis, Familial Mediterranean fever (FMF), HIDS (hyperimmunoglobulinemia D and periodic fever syndrome), TRAPS (TNF-alpha receptor associated periodic fever syndrome), inflammatory bowel disease, Crohn's Disease, ulcerative colitis, recurrent fever, anemia, leukocytosis, asthma, chronic obstructive pulmonary disease, myalgia; Adult Still's disease, Systemic-onset juvenile idiopathic arthritis, Lupus arthritis, Ankylosing spondylitis, familial Mediterranean fever (FMF), TNF receptor-associated periodic syndrome (TRAPS), hyperimmunoglobulinemia D with periodic fever syndrome (HIDS), Blau syndrome, FCAS, MWS, neonatal-onset multisystem inflammatory disease (NOMID) and cryopyrin-associated periodic syndrome (CAPS), familial cold autoinflammatory syndrome (FCAS); Muckle-Wells syndrome (MWS); neonatal-onset multisystem inflammatory disease (NOMID); chronic infantile neurologic, cutaneous, articular syndrome (CINCA); cryopyrin-associated periodic syndrome (CAPS); pyogenic sterile arthritis, pyoderma gangrenosum, and acne syndrome (PAPA).

In further embodiments, the methods described herein are used to treat an inflammatory disease, which includes, but is not limited to asthma, inflammatory bowel disease, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, and vulvitis.

In yet other embodiments, the methods described herein are used to treat an inflammatory skin condition. Inflammatory skin conditions are those conditions of the skin in which inflammatory cells (e.g., polymorphonuclear neutrophils and lymphocytes) infiltrate the skin with no overt or known infectious etiology. Symptoms of inflammatory skin conditions generally include erythema (redness), edema (swelling), pain, pruritus, increased surface temperature and loss of function. As used herein, inflammatory skin conditions include, but are not limited to, allergic contact dermatitis, urticarial dermatitis, psoriasis, eczema and related conditions, insect bites, erythroderma, mycosis fungoides and related conditions, pyoderma gangrenosum, erythema multiforme, rosacea, onychomycosis, and acne and related conditions, but excluding psoriasis and its related conditions.

In some embodiments, the methods described herein are used to treat an autoimmune disease, which includes, but is not limited to, rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease Sjogren's syndrome, multiple sclerosis, Guillain-Barr syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, and vulvodynia.

In some embodiments, the methods described herein are used to treat heteroimmune conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Chronic inflammation in patients has been linked to cancer development (Coussens et al., Nature, 420, 860-867, 2002). Cancers associated with chronic inflammation include, but are not limited to, lung, esophageal, gastric, pancreatic, cervical, bladder, prostate and colorectal cancers. The role of the inflammatory microenvironment as a causative factor in the etiology of cancer is also supported by findings that regular use of non-steroidal anti-inflammatory drugs (NSAIDs) is associated with a reduced incidence of colorectal, breast and gastric cancer. Pro-inflammatory cytokines are mediators of chronic inflammatory responses, and have effects on malignant processes.

Pro-inflammatory cytokines are involved in carcinogenesis and malignant transformation, tumor growth, invasion and metastasis. Persistent expression of proinflammatory cytokines, in or near tumors, exerts a range of effects, including but not limited to, increasing growth and invasiveness of the malignant cells, metastsis, tumorigenesis, to activation of immune-mediated mechanisms, leading to the destruction of tumor cells and inhibition of tumor growth. IL-1β-transfected tumor cells have been reported to fail to induce effective antitumor immune responses. In several human cancers, local IL-1β expression by the malignant cells or the microenvironment has been associated with aggressive tumor growth and poor prognosis.

In IL-1β-transfected fibrosarcoma cells, an up-regulation of MMP-2 and MMP-9 and TGFβ, genes that are involved in invasiveness, was observed, as opposed to the shut-off of these genes in IL-1α-transfected fibrosarcomas cells. IL-1β is thought to also enhance the invasiveness of already existing tumor cells by switching on angiogenesis and by the induction of inflammatory molecules, such as MMPs, heparanase, chemokines or integrins on the malignant cells or endothelial cells, leading to tumor dissemination and metastasis. IL-1β induces secretion of growth and invasiveness-promoting factors, e.g. matrix metalloproteinases and angiogenic factors (i.e. VEGF and bFGF and ELR-positive CXC chemokines, i.e. IL-8 and MCP-1). (Apte et al., seminars in Cancer Biology, vol. 12, 2002, 277-290).

Secreted IL-1β has been implicated in tumor growth and invasion. Inhibition of IL-1β secretion, e.g. by using selective HDAC8 compounds, in malignant cells, or in the tumor's microenvironment provides a method for cancer therapy.

Thus in one embodiment, selective HDAC8 compounds described herein, are used in cancer therapy. In one embodiment, selective HDAC8 compounds described herein, are used in the treatment of sarcomas. In another embodiment, selective HDAC8 compounds described herein, are used in the treatment of sarcomas selected from among alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, malignant fibrous histiocytoma, neurofibrosarcoma, rhabdomyosarcoma, synovial sarcoma, askin's tumor, ewing's, malignant hemangioendothelioma, malignant schwannoma, osteosarcoma, chondrosarcoma.

Symptoms, diagnostic tests, and prognostic tests for each of the above-mentioned conditions are known. See, e.g., “Harrison's Principles of Internal Medicine©,” 16th ed., 2004, The McGraw-Hill Companies, Inc.

In various embodiments described herein, a subject suffers from more than one condition that is treated by administration of a therapeutically effective amount of a selective HDAC8 inhibitor composition. Thus, it is to be understood that the methods described herein are effective for treating a subject suffering from any combination of health conditions amenable to treatment by administration of a selective HDAC8 inhibitor composition. For example, in some embodiments, a subject suffering from a T-cell lymphoma also suffers from an inflammatory condition and vice versa.

### Compounds

Compounds described herein, pharmaceutically acceptable salts, pharmaceutically acceptable N-oxides, pharmaceutically active metabolites, pharmaceutically acceptable prodrugs, or pharmaceutically acceptable solvates thereof, inhibit HDAC8 activity, and are used to treat patients where inhibition of HDAC8 activity provides benefit. Compounds described herein are HDAC8 inhibitor compounds.

In some embodiments of the methods described herein, the selective HDAC8 inhibitor has an IC₅₀ for HDAC8 that is at least about 10 fold lower than the IC₅₀ for HDAC1, HDAC2, HDAC3, HDAC6, HDAC10, or HDAC11. In some embodiments of any of the methods described herein, the selective HDAC8 inhibitor has an IC₅₀ for HDAC8 that is less than about 100 nM and that is at least about 10 fold lower than the IC₅₀ for HDAC1, HDAC2, HDAC3, HDAC6, HDAC10, or HDAC11. In some embodiments of any of the methods described herein, the selective HDAC8 inhibitor has an IC₅₀ for HDAC8 that is less than about 50 nM and that is at least about 10 fold lower than the IC₅₀ of the selective inhibitor for HDAC1, HDAC2, HDAC3, HDAC6, HDAC10, or HDAC11.

In some embodiments, selective HDAC8 inhibitors described herein have an IC₅₀ for HDAC8 that is at least about 15 fold lower than the IC₅₀ for HDAC1, HDAC2, HDAC3, HDAC6, and HDAC10. In some embodiments, selective HDAC8 inhibitors described herein have an IC₅₀ for HDAC8 that is at least about 20 fold lower than the IC₅₀ for HDAC1, HDAC2, HDAC3, HDAC6, and HDAC10. In some embodiments, selective HDAC8 inhibitors described herein have an IC₅₀ for HDAC8 that is at least about 100 fold lower than the IC₅₀ for HDAC1, HDAC2, HDAC3, HDAC6, and HDAC10. In addition, selective HDAC8 inhibitors described herein have an IC₅₀ for HDAC8 that is less than about 100 nM while the IC₅₀ for HDAC1, HDAC2, HDAC3, HDAC6, and HDAC10 is greater than about 100 nM.

In some embodiments, selective HDAC8 inhibitors described herein have an IC₅₀ for HDAC8 that is at least about 10 fold lower than the IC₅₀ for HDAC1. In some embodiments, selective HDAC8 inhibitors described herein have an IC₅₀ for HDAC8 that is at least about 20 fold lower than the IC₅₀ for HDAC1. In some embodiments, selective HDAC8 inhibitors described herein have an IC₅₀ for HDAC8 that is at least about 40 fold lower than the IC₅₀ for HDAC1. In some embodiments, selective HDAC8 inhibitors described herein have an IC₅₀ for HDAC8 that is at least about 100 fold lower than the IC₅₀ for HDAC1. In some embodiments, selective HDAC8 inhibitors described herein have an IC₅₀ for HDAC8 that is at least about 150 fold lower than the IC₅₀ for HDAC1. In yet other embodiments, selective HDAC8 inhibitors described herein have an IC₅₀ for HDAC8 that is at least about 200 fold lower than the IC₅₀ for HDAC1.

In some embodiments, selective HDAC8 inhibitors described herein have IC₅₀ for HDAC8 that is less than about 100 nM and that is at least about 20 fold lower than the IC₅₀ for other HDAC isoforms (HDAC1, HDAC2, HDAC3, HDAC6, HDAC10), wherein the IC₅₀ for the other HDAC isoforms is greater than about 100 nM.

In one embodiment, described herein are selective histone deacetylase 8 (HDAC8) inhibitors. In one embodiment, the selective HDAC8 inhibitor has an IC₅₀ for histone deacetylase 8 activity that is at least about 10 fold lower than the IC₅₀ of the selective HDAC8 inhibitor for activity of histone deacetylase 1, histone deacetylase 2, histone deacetylase 3, histone deacetylase 6, histone deacetylase 10, or histone deacetylase 11.

In one aspect is a compound having a structure of Formula (I):

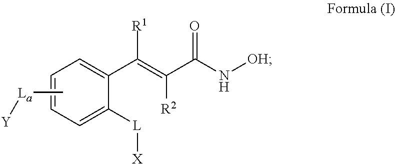

wherein:
R¹ and R² are each independently H, OH, halogen, or C₁-C₆alkyl;
L and Lₐ are each independently a bond, O, S, NR³, —NR¹⁰C(═O)—R¹¹, S(═O), S(═O)₂, NHS(═O)₂, —C₁-C₆alkylene-, —C₂-C₆alkenylene-, —C₂-C₆alkynylene-, —C₁-C₆heteroalkylene-, —C₁-C₆alkylene-O—, —C₁-C₃alkylene-O—C₁-C₃alkylene-, —C₁-C₆ alkylene-NR³—, —C₁-C₃alkylene-NR³—C₁-C₃alkylene-, —C₁-C₆alkylene-C(═O)NR³—, —C₁-C₃alkylene-C(═O)NR³—C₁-C₃alkylene-, —C₁-C₆alkylene-NR³C(═O)—, —C₁-C₃ alkylene-NR³C(═O)—C₁-C₃alkylene-, —C₁-C₆alkylene-S—, —C₁-C₃alkylene-S—C₁-C₃ alkylene-, —C₁-C₆alkylene-S(═O)—, —C₁-C₃alkylene-S(═O)—C₁-C₃alkylene, —C₁-C₆alkylene-S(═O)₂—, —C₁-C₃alkylene-S(═O)₂—C₁-C₃alkylene, —C(═O)—, or —C(═O)—C₁-C₆ alkylene;
X is a substituted or unsubstituted group selected from among aryl, heteroaryl, C₃-C₁₀cycloalkyl, and C₂-C₁₀heterocycloalkyl; where if X is substituted, then X is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR ° S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
Y is H or a substituted or unsubstituted group selected from among C₁-C₆alkyl, —CO₂R¹⁰, —C(═O)R¹¹, —NR¹⁰C(═O)—R¹¹, —C(═O)N(R¹⁰)₂ aryl, heteroaryl, C₃-C₁₀cycloalkyl, and C₂-C₁₀heterocycloalkyl; where if Y is substituted, then Y is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)₂—R¹¹, —S(═O)₂—R¹¹, —NR—R¹¹, —C(═O)N(R¹⁰)₂, —S(═O)₂N(R¹⁰)₂, —NR¹⁰S(═O)₂—R¹¹, —OC(═O)N(R¹⁰)₂—NR¹⁰C(═O)O—R¹¹, —OC(═O)O—R¹¹, —NHC(═O)NH—R¹¹—OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
   R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
   R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is H, C₁-C₆alkyl, phenyl or benzyl;

or a pharmaceutically acceptable salt, pharmaceutically acceptable N-oxide, or pharmaceutically acceptable prodrug thereof.

In one embodiment, is a substituted cinnamic acid hydroxyamide compound, wherein the substituent at the 2-position is L-X, wherein:
L is a bond, O, S, NR³, S(═O), S(═O)₂, —C₁-C₆alkylene-, —C₂-C₆alkenylene-, —C₂-C₆alkynylene-, —C₁-C₆heteroalkylene-, —C₁-C₆alkylene-O—, —C₁-C₃alkylene-O—C₁-C₃alkylene-, —C₁-C₆alkylene-NR³—, —C₁-C₃alkylene-NR³—C₁-C₃alkylene-, —C₁-C₆alkylene-C(═O)NR³—, —C₁-C₃alkylene-C(═O)NR³—C₁-C₃alkylene-, —C₁-C₆alkylene-NR³C(═O)—, —C₁-C₃alkylene-NR³C(═O)—C₁-C₃alkylene-, —C₁-C₆alkylene-S—, —C₁-C₃alkylene-S—C₁-C₃alkylene-, —C₁-C₆alkylene-S(═O)—, —C₁-C₃alkylene-S(═O)—C₁-C₃alkylene, —C₁-C₆alkylene-S(═O)₂—, —C₁-C₃alkylene-S(═O)₂—C₁-C₃alkylene, —C(═O)—, or —C(═O)—C₁-C₆alkylene;
X is a substituted or unsubstituted group selected from among aryl, heteroaryl, C₃-C₁₀cycloalkyl, and C₂-C₁₀heterocycloalkyl; where if X is substituted, then X is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
   or an active metabolite, pharmaceutically acceptable solvate, pharmaceutically acceptable salt, pharmaceutically acceptable N-oxide, or pharmaceutically acceptable prodrug thereof.

For any and all embodiments, substituents are selected from among a subset of the listed alternatives. For example, in some embodiments, L is O, S, or NR³. In other embodiments, L is O. In some embodiments, L is S. In some embodiments, L is NR³ wherein R³ is hydrogen. In one embodiment, L is NR³ wherein R³ is C₁-C₆alkyl. In another embodiment, L is NR³ wherein R³ is methyl, ethyl, n-propyl, iso-propyl, n-butyl, iso-butyl, or tert-butyl. In another embodiment, L is NR³ wherein R³ is methyl. In yet another embodiment, L is a bond. In yet another embodiment, L is S(═O) or S(═O)₂. In another embodiment, L is —C₁-C₆alkylene-, —C₂-C₆alkenylene-, or —C₂-C₆alkynylene-. In yet another embodiment, L is C₁-C₆alkylene selected from methylene, ethylene or propylene. In another embodiment, L is —C₁-C₆heteroalkylene-, —C₁-C₆alkylene-O—, —C₁-C₃alkylene-O—C₁-C₃alkylene-, —C₁-C₆alkylene-NR³—, —C₁-C₆alkylene-S—, —C₁-C₃alkylene-S—C₁-C₃alkylene-, or —C₁-C₃alkylene-NR³—C₁-C₃alkylene-. In one embodiment, L is —CH₂—S—. In another embodiment, L is —CH₂NR³—. In yet a further embodiment, L is —CH₂NR³ wherein R³ is H. In one embodiment, L is —CH₂—O—. In yet another embodiment, L is —C₁-C₆alkylene-C(═O)NR³—, —C₁-C₃alkylene-C(═O)NR³—C₁-C₃alkylene-, —C₁-C₆alkylene-NR³C(═O)—, or —C₁-C₃alkylene-NR³C(═O)—C₁-C₃alkylene-. In yet another embodiment, L is —C₁-C₆alkylene-S(═O)—, —C₁-C₃alkylene-S(═O)—C₁-C₃alkylene, —C₁-C₆alkylene-S(═O)₂—, —C₁-C₃alkylene-S(═O)₂—C₁-C₃alkylene. In yet a further embodiment, L is —C(═O)—, or —C(═O)—C₁-C₆alkylene.

Also described herein is a compound of Formula I( ), wherein R¹ and R² are each independently H or C₁-C₆alkyl. In another embodiment, R¹ is H. In yet another embodiment, R² is H. In a further embodiment, both R¹ and R² are H. In yet a further embodiment, R¹ is C₁-C₆alkyl selected from methyl, ethyl, n-propyl, iso-propyl, n-butyl, iso-butyl, tert-butyl, n-pentyl, or n-hexyl. In another embodiment, R² is C₁-C₆alkyl selected from methyl, ethyl, n-propyl, iso-propyl, n-butyl, iso-butyl, tert-butyl, n-pentyl, or n-hexyl.

In another embodiment, is a compound of Formula (II):

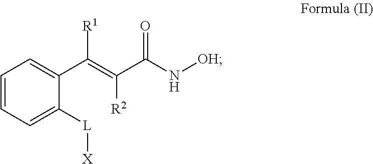

wherein:
R¹ and R² are each independently H, OH, halogen, or C₁-C₆alkyl;
L is a bond, O, S, NR³, —NR¹⁰C(═O)—R¹¹, S(═O), S(═O)₂, —C₁-C₆alkylene-, —C₂-C₆alkenylene-, —C₂-C₆alkynylene-, —C₁-C₆heteroalkylene-, —C₁-C₆alkylene-O—, —C₁-C₃alkylene-O—C₁-C₃alkylene-, —C₁-C₆alkylene-NR³—, —C₁-C₃alkylene-NR³—C₁-C₃alkylene-, —C₁-C₆alkylene-C(═O)NR³—, —C₁-C₃alkylene-C(═O)NR³—C₁-C₃alkylene-, —C₁-C₆alkylene-NR³C(═O)—, —C₁-C₃alkylene-NR³C(═O)—C₁-C₃alkylene-, —C₁-C₆alkylene-S—, —C₁-C₃alkylene-S—C₁-C₃alkylene-, —C₁-C₆alkylene-S(═O)—, —C₁-C₃alkylene-S(═O)—C₁-C₃alkylene, —C₁-C₆alkylene-S(═O)₂—, —C₁-C₃alkylene-S(═O)₂—C₁-C₃alkylene, —C(═O)—, or —C(═O)—C₁-C₆alkylene;
X is a substituted or unsubstituted group selected from among aryl, heteroaryl, C₃-C₁₀cycloalkyl, and C₂-C₁₀heterocycloalkyl; where if X is substituted, then X is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is H, C₁-C₆alkyl, phenyl or benzyl;
   or a pharmaceutically acceptable salt, pharmaceutically acceptable N-oxide, or pharmaceutically acceptable prodrug thereof.

In another embodiment, is a compound of Formula (IIA):

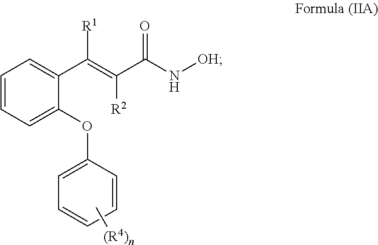

wherein:

R¹ and R² are each independently H, OH, halogen, or C₁-C₆alkyl;
R⁴ is selected from among hydrogen, halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
n is an integer from 0 to 5;
   R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
   R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
      or a pharmaceutically acceptable salt, pharmaceutically acceptable N-oxide, or pharmaceutically acceptable prodrug thereof.

In one embodiment is a compound of Formula (IIA) wherein R¹ and R² are each independently H. In another embodiment is a compound of Formula (IIA) wherein R⁴ is halogen. In another embodiment, R⁴ is Cl. In a further embodiment, R⁴ is F. In another embodiment, R⁴ is Br. In another embodiment R⁴ is C₁-C₆alkyl. In yet another embodiment, R⁴ is methyl, ethyl, n-propyl, iso-propyl, iso-butyl, and tert-butyl. In a further embodiment, R⁴ is methyl. In one embodiment, R⁴ is C₁-C₆alkoxy. In another embodiment, R⁴ is methoxy. In a further embodiment, R⁴ is ethoxy. In another embodiment is a compound of Formula (IIA) wherein n is 1. In a further embodiment, n is 2. In yet a further embodiment R⁴ is substituted para to the ether linker. In another embodiment, R⁴ is substituted meta to the ether linker. In yet another embodiment, R⁴ is substituted ortho to the ether linker

In another embodiment, is a compound of Formula (IIB):

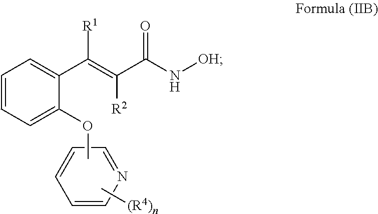

wherein:
R¹ and R² are each independently H, OH, halogen, or C₁-C₆alkyl;
R⁴ is selected from among hydrogen, halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈ cycloalkyl, substituted or unsubstituted C₂-C₁₀heterocycloalkyl, substituted or unsubstituted aryl, and substituted or unsubstituted heteroaryl;
n is an integer from 0 to 4;
   R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
   R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
      or a pharmaceutically acceptable salt, pharmaceutically acceptable N-oxide, or pharmaceutically acceptable prodrug thereof.

In one embodiment is a compound of Formula (IIB) wherein R¹ and R² are each independently H. In one embodiment is a compound of Formula (IIB) wherein R⁴ is hydrogen. In another embodiment is a compound of Formula (IIB) wherein R⁴ is halogen. In another embodiment, R⁴ is Cl. In a further embodiment, R⁴ is F. In another embodiment, R⁴ is Br. In another embodiment R⁴ is C₁-C₆alkyl. In yet another embodiment, R⁴ is methyl, ethyl, n-propyl, iso-propyl, iso-butyl, and tert-butyl. In a further embodiment, R⁴ is methyl. In one embodiment, R⁴ is C₁-C₆alkoxy. In another embodiment, R⁴ is methoxy. In a further embodiment, R⁴ is ethoxy. In another embodiment is a compound of Formula (IIA) wherein n is 1. In a further embodiment, n is 2. In yet a further embodiment R⁴ is substituted para to the ether linker. In another embodiment, R⁴ is substituted meta to the ether linker. In yet another embodiment, R⁴ is substituted ortho to the ether linker

In another embodiment, is a compound of Formula (IIC):

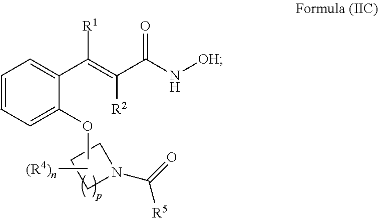

wherein:
R¹ and R² are each independently H, OH, halogen, or C₁-C₆alkyl;
R⁴ and R⁵ are each independently selected from among hydrogen, halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
p is an integer from 0 to 4;
n is an integer from 0 to 5;
   R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
   R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
      or a pharmaceutically acceptable salt, pharmaceutically acceptable N-oxide, or pharmaceutically acceptable prodrug thereof.

In one embodiment is a compound of Formula (IIC) wherein R¹ and R² are each independently H. In another embodiment is a compound of Formula (IIC) wherein p is 2. In another embodiment is a compound of Formula (IIC) wherein p is 3. In a further embodiment is a compound of Formula (IIC) wherein p is 4.

In one embodiment is a compound selected from Formula (IID):

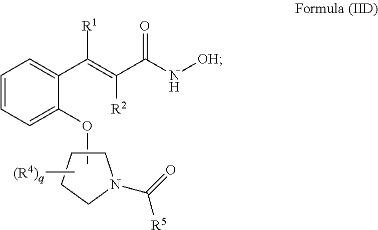

wherein:
R¹ and R² are each independently H, OH, halogen, or C₁-C₆alkyl;
R⁴ and R⁵ are each independently selected from among hydrogen, halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
q is an integer from 0 to 3;
   R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
   R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
      or a pharmaceutically acceptable salt, pharmaceutically acceptable N-oxide, or pharmaceutically acceptable prodrug thereof.

In one embodiment is a compound of Formula (IID) wherein q is 0. In another embodiment is a compound of Formula (IID) wherein R¹ and R² are each independently H. In a further embodiment is a compound of Formula (IID) wherein R⁵ is C₁-C₆alkyl. In another embodiment, R⁵ is methyl, ethyl, n-propyl, iso-propyl, n-butyl, iso-butyl, or tert-butyl. In a further embodiment R⁵ is methyl. In yet a further embodiment, R⁵ is iso-propyl. In yet a further embodiment, R⁵ is aryl. In another embodiment, R⁵ is phenyl. In yet another embodiment, R⁵ is heteroaryl. In one embodiment, R⁵ is selected from pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, 4-azaindolyl, 5-azaindolyl, 6-azaindolyl, 7-azaindolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, imidazo[1,2-a]pyridinyl, thiophenopyridinyl, and furopyridinyl. In another embodiment, R⁵ is pyridinyl. In a further embodiment, R⁵ is furyl. In a further embodiment, R⁵ is thienyl.

In another embodiment is a compound of Formula (IIE):

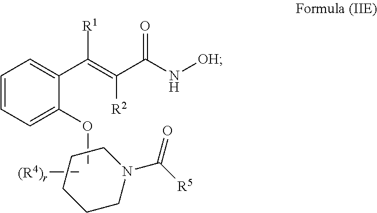

wherein:
R¹ and R² are each independently H, OH, halogen, or C₁-C₆alkyl;
R⁴ and R⁵ are each independently selected from among hydrogen, halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹)₂, —S(═O)₂N(R¹)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
r is an integer from 0 to 4;
   R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
   R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
      or a pharmaceutically acceptable salt, pharmaceutically acceptable N-oxide, or pharmaceutically acceptable prodrug thereof.

In one embodiment is a compound of Formula (IIE) wherein r is 0. In another embodiment is a compound of Formula (IIE) wherein R¹ and R² are each independently H. In a further embodiment is a compound of Formula (IIE) wherein R⁵ is C₁-C₆alkyl. In another embodiment, R⁵ is methyl, ethyl, n-propyl, iso-propyl, n-butyl, iso-butyl, or tert-butyl. In a further embodiment R⁵ is methyl. In yet a further embodiment, R⁵ is aryl. In another embodiment, R⁵ is phenyl. In yet another embodiment, R⁵ is heteroaryl. In one embodiment, R⁵ is selected from pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, 4-azaindolyl, 5-azaindolyl, 6-azaindolyl, 7-azaindolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, imidazo[1,2-a]pyridinyl, thiophenopyridinyl, and furopyridinyl. In another embodiment, R⁵ is pyridinyl. In a further embodiment, R⁵ is furyl. In a further embodiment, R⁵ is thienyl.

In yet another embodiment is a compound of Formula (IIF):

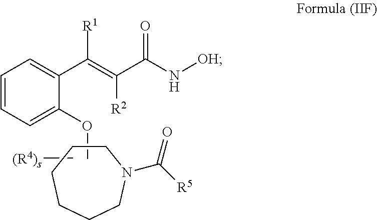

wherein:
R¹ and R² are each independently H, OH, halogen, or C₁-C₆alkyl;
R⁴ and R⁵ are each independently selected from among hydrogen, halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
s is an integer from 0 to 5;
   R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
   R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
      or a pharmaceutically acceptable salt, pharmaceutically acceptable N-oxide, or pharmaceutically acceptable prodrug thereof.

In one embodiment is a compound of Formula (IIF) wherein s is 0. In another embodiment is a compound of Formula (IIF) wherein R¹ and R² are each independently H. In a further embodiment is a compound of Formula (IIF) wherein R⁵ is C₁-C₆alkyl. In another embodiment, R⁵ is methyl, ethyl, n-propyl, iso-propyl, n-butyl, iso-butyl, or tert-butyl. In a further embodiment R⁵ is methyl. In yet a further embodiment, R⁵ is aryl. In another embodiment, R⁵ is phenyl. In yet another embodiment, R⁵ is heteroaryl. In one embodiment, R⁵ is selected from pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, 4-azaindolyl, 5-azaindolyl, 6-azaindolyl, 7-azaindolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, imidazo[1,2-a]pyridinyl, thiophenopyridinyl, and furopyridinyl. In another embodiment, R⁵ is pyridinyl. In a further embodiment, R⁵ is furyl. In a further embodiment, R⁵ is thienyl.

In yet another embodiment is a compound of Formula (III):

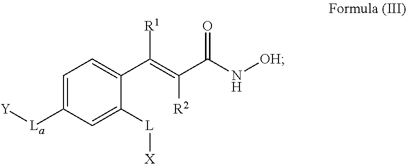

wherein:
R¹ and R² are each independently H, OH, halogen, or C₁-C₆alkyl;
L and Lₐ are each independently a bond, O, S, NR³, —NR¹⁰C(═O)—R¹¹, S(═O), S(═O)₂, NHS(═O)₂, —C₁-C₆alkylene-, —C₂-C₆alkenylene-, —C₂-C₆alkynylene-, —C₁-C₆heteroalkylene-, —C₁-C₆alkylene-O—, —C₁-C₃alkylene-O—C₁-C₃alkylene-, —C₁-C₆ alkylene-NR³—, —C₁-C₃alkylene-NR³—C₁-C₃alkylene-, —C₁-C₆alkylene-C(═O)NR³—, —C₁-C₃alkylene-C(═O)NR³—C₁-C₃alkylene-, —C₁-C₆alkylene-NR³C(═O)—, —C₁-C₃ alkylene-NR³C(═O)—C₁-C₃alkylene-, —C₁-C₆alkylene-S—, —C₁-C₃alkylene-S—C₁-C₃ alkylene-, —C₁-C₆alkylene-S(═O)—, —C₁-C₃alkylene-S(═O)—C₁-C₃alkylene, —C₁-C₆alkylene-S(═O)₂—, —C₁-C₃alkylene-S(═O)₂—C₁-C₃alkylene, —C(═O)—, or —C(═O)—C₁-C₆ alkylene;
X is a substituted or unsubstituted group selected from among aryl, heteroaryl, C₃-C₁₀cycloalkyl, and C₂-C₁₀heterocycloalkyl; where if X is substituted, then X is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
Y is H or a substituted or unsubstituted group selected from among C₁-C₆alkyl, —CO₂R¹⁰, —C(═O)R¹¹, —NR¹⁰C(O)—R¹¹, —C(═O)N(R¹⁰)₂, aryl, heteroaryl, C₃-C₁₀cycloalkyl, and C₂-C₁₀heterocycloalkyl; where if Y is substituted, then Y is substituted with 1, 2, 3, 4, or 5 groups selected from among halogen, C₁-C₆alkoxy, C₁-C₆fluoroalkoxy, aminoC₁-C₆ alkoxy, C₁-C₃ alkylamino C₁-C₃ alkoxy, hydroxyC₁-C₃ alkylamino C₁-C₃ alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —C(═O)O—R¹¹, —NHC(═O)NH—R¹¹—OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈ cycloalkyl, substituted or unsubstituted C₂-C₁₀heterocycloalkyl, substituted or unsubstituted aryl, and substituted or unsubstituted heteroaryl;

R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈ cycloalkyl, C₂-C₈ heterocycloalkyl, aryl, and heteroaryl;
R³ is H, C₁-C₆alkyl, phenyl or benzyl;
   or a pharmaceutically acceptable salt, pharmaceutically acceptable N-oxide, or pharmaceutically acceptable prodrug thereof.

In yet another embodiment is a compound of Formula (IV):

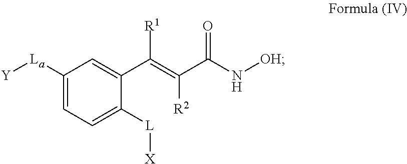

wherein:
R¹ and R² are each independently H, OH, halogen, or C₁-C₆alkyl; L and Lₐ are each independently a bond, O, S, NR³, —NR¹⁰C(═O)—R¹, S(═O), S(═O)₂, NHS(═O)₂, —C₁-C₆alkylene-, —C₂-C₆alkenylene-, —C₂-C₆alkynylene-, —C₁-C₆heteroalkylene-, —C₁-C₆alkylene-O—, —C₁-C₃alkylene-O—C₁-C₃alkylene-, —C₁-C₆ alkylene-NR³—, —C₁-C₃alkylene-NR³—C₁-C₃alkylene-, —C₁-C₆alkylene-C(═O)NR³—, —C₁-C₃alkylene-C(═O)NR³—C₁-C₃alkylene-, —C₁-C₆alkylene-NR³C(═O)—, —C₁-C₃ alkylene-NR³C(═O)—C₁-C₃alkylene-, —C₁-C₆alkylene-S—, —C₁-C₃alkylene-S—C₁-C₃ alkylene-, —C₁-C₆alkylene-S(═O)—, —C₁-C₃alkylene-S(═O)—C₁-C₃alkylene, —C₁-C₆alkylene-S(═O)₂—, —C₁-C₃alkylene-S(═O)₂—C₁-C₃alkylene, —C(═O)—, or —C(═O)—C₁-C₆ alkylene;
X is a substituted or unsubstituted group selected from among aryl, heteroaryl, C₃-C₁₀cycloalkyl, and C₂-C₁₀heterocycloalkyl; where if X is substituted, then X is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
Y is H or a substituted or unsubstituted group selected from among C₁-C₆alkyl, —CO₂R¹⁰, —C(═O)R¹¹, —NR¹⁰C(═O)—R¹¹, —C(═O)N(R¹⁰)₂, S(═O)₂—R¹¹, aryl, heteroaryl, C₃-C₁₀cycloalkyl, and C₂-C₁₀heterocycloalkyl; where if Y is substituted, then Y is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂,—S(═O)₂N(R¹⁰)₂, —NR—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂), —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
   R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
   R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is H, C₁-C₆alkyl, phenyl or benzyl;
or a pharmaceutically acceptable salt, pharmaceutically acceptable N-oxide, or pharmaceutically acceptable prodrug thereof.

In another embodiment is a compound of Formula (III) wherein, Lₐ is a bond. In one embodiment, Lₐ is 0. In a further embodiment, Lₐ is NH.

In another embodiment is a compound of Formula (Ma) having the structure:

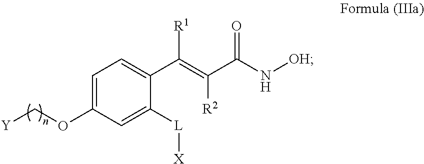

wherein:
R¹ and R² are each independently H, OH, halogen, or C₁-C₆alkyl;
L is a bond, O, S, NR³, —NR¹⁰C(═O)—R¹¹, S(═O), S(═O)₂, —C₁-C₆alkylene-, —C₂-C₆alkenylene-, —C₂-C₆alkynylene-, —C₁-C₆heteroalkylene-, —C₁-C₆alkylene-O—, —C₁-C₃alkylene-O—C₁-C₃alkylene-, —C₁-C₆alkylene-NR³—, —C₁-C₃alkylene-NR³—C₁-C₃alkylene-, —C₁-C₆alkylene-C(═O)NR³—, —C₁-C₃alkylene-C(═O)NR³—C₁-C₃alkylene-, —C₁-C₆alkylene-NR³C(═O)—, —C₁-C₃alkylene-NR³C(═O)—C₁-C₃alkylene-, —C₁-C₆alkylene-S—, —C₁-C₃alkylene-S—C₁-C₃alkylene-, —C₁-C₆alkylene-S(═O)—, —C₁-C₃alkylene-S(═O)—C₁-C₃alkylene, —C₁-C₆alkylene-S(═O)₂—, —C₁-C₃alkylene-S(═O)₂—C₁-C₃alkylene, —C(═O)—, or —C(═O)—C₁-C₆alkylene;
X is a substituted or unsubstituted group selected from among aryl, heteroaryl, C₃-C₁₀cycloalkyl, and C₂-C₁₀heterocycloalkyl; where if X is substituted, then X is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
Y is H or a substituted or unsubstituted group selected from among C₁-C₆alkyl, —CO₂R¹⁰, —C(═O)R¹¹, —NR¹⁰C(═O)—R¹¹, —C(═O)N(R¹⁰)₂, aryl, heteroaryl, C₃-C₁₀cycloalkyl, and C₂-C₁₀heterocycloalkyl; where if Y is substituted, then Y is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈ cycloalkyl, substituted or unsubstituted C₂-C₁₀heterocycloalkyl, substituted or unsubstituted aryl, and substituted or unsubstituted heteroaryl;
   R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
   R¹¹ is a substituted or unsubstituted group selected from among C₁-C₆alkyl, C₁-C₆fluoroalkyl, C₃-C₈ cycloalkyl, C₂-C₈ heterocycloalkyl, aryl, and heteroaryl;
   n is an integer from 0 to 4;
   R³ is H, C₁-C₆alkyl, phenyl or benzyl;
      or a pharmaceutically acceptable salt, pharmaceutically acceptable N-oxide, or pharmaceutically acceptable prodrug thereof.

In another embodiment is a compound having the structure:

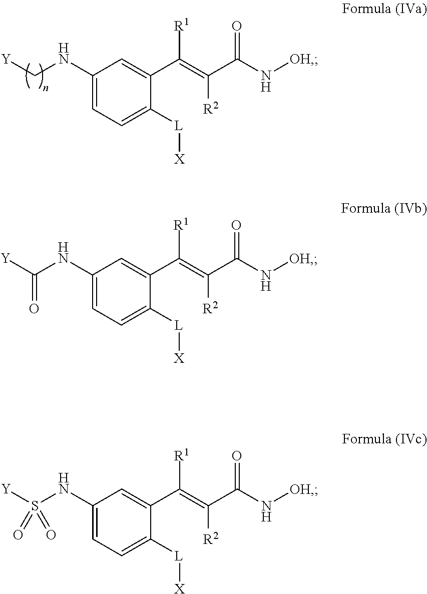

wherein:
R¹ and R² are each independently H, OH, halogen, or C₁-C₆alkyl;
L is a bond, O, S, NR³, —NR¹⁰C(═O)—R¹, S(═O), S(═O)₂, —C₁-C₆alkylene-, —C₂-C₆ alkenylene-, —C₂-C₆alkynylene-, —C₁-C₆heteroalkylene-, —C₁-C₆alkylene-O—, —C₁-C₃ alkylene-O—C₁-C₃alkylene-, —C₁-C₆alkylene-NR³⁻⁵—C₁-C₃alkylene-NR³—C₁-C₃ alkylene-, —C₁-C₆alkylene-C(═O)NR³, —C₁-C₃alkylene-C(═O)NR³—C₁-C₃alkylene-, —C₁-C₆alkylene-NR³C(═O)—, —C₁-C₃alkylene-NR³C(═O)—C₁-C₃alkylene-, —C₁-C₆ alkylene-S—, —C₁-C₃alkylene-S—C₁-C₃alkylene-, —C₁-C₆alkylene-S(═O)—, —C₁-C₃ alkylene-S(═O)—C₁-C₃alkylene, —C₁-C₆alkylene-S(═O)₂—, —C₁-C₃alkylene-S(═O)₂—C₁-C₃alkylene, —C(═O)—, or —C(═O)—C₁-C₆alkylene;
X is a substituted or unsubstituted group selected from among aryl, heteroaryl, C₃-C₁₀cycloalkyl, and C₂-C₁₀heterocycloalkyl; where if X is substituted, then X is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
Y is H or a substituted or unsubstituted group selected from among C₁-C₆alkyl, —CO₂R¹⁰, —C(═O)R¹¹, —NR¹⁰C(═O)—R¹¹, —C(═O)N(R¹⁰)₂, aryl, heteroaryl, C₃-C₁₀cycloalkyl, and C₂-C₁₀heterocycloalkyl; where if Y is substituted, then Y is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
   R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
   R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
n is an integer from 0 to 4;
R³ is H, C₁-C₆alkyl, phenyl or benzyl;
or a pharmaceutically acceptable salt, pharmaceutically acceptable N-oxide, or pharmaceutically acceptable prodrug thereof.

In one embodiment is a compound of Formula (I), (II), (III), (IIIa), (IV), (IVa), (IVb), or (IVc), wherein X is a substituted or unsubstituted group selected from among aryl, heteroaryl, C₃-C₁₀cycloalkyl, and C₂-C₁₀heterocycloalkyl; where if X is substituted, then X is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl.

In some embodiments is a compound of Formula (I), (II), (III), (IIIa), (IV), (IVa), (IVb), or (IVc), wherein X is a substituted or unsubstituted aryl group. In another embodiment, X is a substituted or unsubstituted phenyl group. In yet another embodiment, X is a substituted or unsubstituted naphthalene group. In yet a further embodiment, X is an unsubstituted phenyl group.

In some embodiments, X is selected from among phenyl, 2-methylphenyl, 3-methylphenyl, 4-methylphenyl, 3,4-dimethylphenyl, 2-fluorophenyl, 3-fluorophenyl, 4-fluorophenyl, 3,4-difluorophenyl, 2-chlorophenyl, 3-chlorophenyl, 4-chlorophenyl, 2,4-dichlorophenyl, 3,4-dichlorophenyl, 3-methoxyphenyl, 4-methoxyphenyl, 3,5-dimethoxyphenyl, 3,4,5-trimethoxyphenyl, 2-(trifluoromethyl)-phenyl, 3-(trifluoromethyl)-phenyl, 4-(trifluoromethyl)-phenyl, 2-(trifluoromethoxy)-phenyl, 3-(trifluoromethoxy)-phenyl, 4-(trifluoromethoxy)-phenyl, 2-chloro-4-fluorophenyl, 3-chloro-4-fluorophenyl, 2-fluoro-4-chlorophenyl, 3-fluoro-4-cholorophenyl, 2-chloro-4-methoxyphenyl, 2,3-dichlorophenyl, 3-methoxy-4-fluorophenyl, 3-methoxy-5-fluorophenyl, 3-methoxy-4-chlorophenyl, 3-(methylsulfonyl)phenyl, 4-(methylsulfonyl)phenyl, 2-thiophenyl, 3-thiophenyl, 2,3-difluorophenyl, 2,4-difluorophenyl, 3-fluoro-4-methoxy-phenyl, 2-(difluoromethoxy)-phenyl, 3-(difluoromethoxy)-phenyl, 4-(difluoromethoxy)-phenyl, N-methylsulfonyl-2-aminophenyl, N-methylsulfonyl-3-aminophenyl, N-methylsulfonyl-4-aminophenyl, N-phenylsulfonyl-2-aminophenyl, N-phenylsulfonyl-3-aminophenyl, N-phenylsulfonyl-4-aminophenyl, 2-nitrophenyl, 3-nitrophenyl, 4-nitrophenyl, 2-aminophenyl, 3-aminophenyl, 4-aminophenyl, 2-dimethylaminophenyl, 3-dimethylaminophenyl, 4-dimethylaminophenyl, N-acetyl-2-aminophenyl, N-acetyl-3-aminophenyl, N-acetyl-4-aminophenyl, N-benzoyl-2-aminophenyl, N-benzoyl-3-aminophenyl, and N-benzoyl-4-aminophenyl.

In other embodiments, X is selected from among phenyl, 3-methoxyphenyl, 4-methoxyphenyl, 2-methylphenyl, 3-methylphenyl, 4-methylphenyl, 2-fluorophenyl, 3-fluorophenyl, 4-fluorophenyl, 2-chlorophenyl, 3-chlorophenyl, 4-chlorophenyl, 3-fluoro-4-methoxyphenyl, 4-(trifluoromethoxy)-phenyl, 3,4-dichlorophenyl, 2,4-dichlorophenyl, 2-chloro-4-fluorophenyl, 3-chloro-4-fluorophenyl, 2-fluoro-4-chlorophenyl, 3-fluoro-4-cholorophenyl, 2-chloro-4-methoxyphenyl, 2,3-dichlorophenyl, 3-methoxy-4-fluorophenyl, 3-methoxy-5-fluorophenyl, 3-methoxy-4-chlorophenyl, 3-(methylsulfonyl)phenyl, 4-(methylsulfonyl)phenyl, 2-thiophenyl, 3-thiophenyl, 2,3-difluorophenyl, 2,4-difluorophenyl, and 3,4-difluorophenyl.

In another embodiment is a compound of Formula (I), (II), (III), (IIIa), (IV), (IVa), (IVb), or (IVc), wherein X is a substituted or unsubstituted heteroaryl. In one embodiment X is heteroaryl selected from pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, 4-azaindolyl, 5-azaindolyl, 6-azaindolyl, 7-azaindolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, imidazo[1,2-a]pyridinyl, thiophenopyridinyl, and furopyridinyl. In one embodiment, X is a substituted or unsubstituted 2-pyridyl, 3-pyridyl, or 4-pyridyl. In another embodiment, 2-pyridyl, 3-pyridyl, 4-pyridyl are each independently substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl. In a further embodiment, 2-pyridyl, 3-pyridyl, 4-pyridyl are each independently substituted with 1, 2, 3, 4, or 5 groups selected from among halogen, C₁-C₆alkoxy, —CN, —NO₂, —CO₂R¹⁰, —C(═O)R¹¹, and —S—R¹¹. In one embodiment, halogen is Cl. In another embodiment, halogen is Br. In a further embodiment, halogen is F. In yet a further embodiment, 2-pyridyl, 3-pyridyl, 4-pyridyl are each independently substituted with CN. In yet another embodiment, 2-pyridyl, 3-pyridyl, 4-pyridyl are each independently substituted with OH. In yet a further embodiment, 2-pyridyl, 3-pyridyl, 4-pyridyl are each independently substituted with at least two substituents. In a further embodiment, 2-pyridyl, 3-pyridyl, 4-pyridyl are each independently substituted with at least three substituents.

Also described herein is a compound of Formula (I), (II), (III), (Ma), (IV), (IVa), (IVb), or (IVc), wherein X is a substituted or unsubstituted C₃-C₈cycloalkyl. In one embodiment C₃-C₈cycloalkyl is selected from cyclopentyl, cyclohexyl, and cycloheptyl. In one embodiment, cyclopentyl, cyclohexyl, and cycloheptyl are each independently substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(O)R¹¹, —S—R¹¹, —S(═O)—R¹¹, S(═O)₂—R¹¹, —NR¹⁰C(═O)—R¹¹—C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl. In a further embodiment, cyclopentyl, cyclohexyl, and cycloheptyl are each independently substituted with 1, 2, 3, 4, or 5 groups selected from among halogen, C₁-C₆alkoxy, —CN, —NO₂, —CO₂R¹⁰, —C(═O)R¹¹, and —S—R¹¹. In one embodiment, halogen is Cl. In another embodiment, halogen is Br. In a further embodiment, halogen is F. In yet a further embodiment, cyclopentyl, cyclohexyl, and cycloheptyl are each independently substituted with CN. In yet another embodiment, cyclopentyl, cyclohexyl, and cycloheptyl are each independently substituted with OH. In yet a further embodiment, cyclopentyl, cyclohexyl, and cycloheptyl are each independently substituted with at least two substituents. In a further embodiment, cyclopentyl, cyclohexyl, and cycloheptyl are each independently substituted with at least three substituents.

In one embodiment is a compound of Formula (I), (II), (III), (IIIa), (IV), (IVa), (IVb), or (IVc), wherein X is substituted or unsubstituted C₂-C₁₀heterocycloalkyl selected from quinolizinyl, dioxinyl, piperidinyl, morpholinyl, thiomorpholinyl, thiazinyl, tetrahydropyridinyl, piperazinyl, oxazinanonyl, dihydropyrrolyl, dihydroimidazolyl, tetrahydrofuranyl, tetrahydropyranyl, dihydrooxazolyl, oxiranyl, pyrrolidinyl, pyrazolidinyl, dihydrothienyl, imidazolidinonyl, pyrrolidinonyl, dihydrofuranonyl, dioxolanonyl, thiazolidinyl, piperidinonyl, indolinyl, tetrahydroquinolinyl, tetrahydroisoquinolinyl, and tetrahydrothienyl. In one embodiment, X is substituted or unsubstituted piperidinyl or pyrrolidine. In one embodiment, is a compound of Formula (I), (II), (III), (IIIa), (IV), (IVa), (IVb), or (IVc), wherein L-X is O-piperidinyl or O-pyrrolidine wherein piperidinyl or pyrrolidine is optionally substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═)₂—R¹¹, —NR¹⁰C(═O)—R¹¹, —C(═O)N(R¹⁰)₂, —S(═O)₂N(R¹⁰)₂, —NR¹⁰S(═O)₂—R¹¹—OC(═O)N(R¹⁰)₂—NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl.

In one embodiment, L-X is

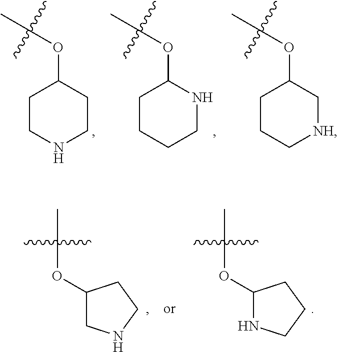 [SMILES: *OC1CCNCC1 ; *OC1CCCCN1 ; *OC1CCCNC1 ; *OC1CCNC1 ; *OC1CCCN1]

In another embodiment, L-X is selected from

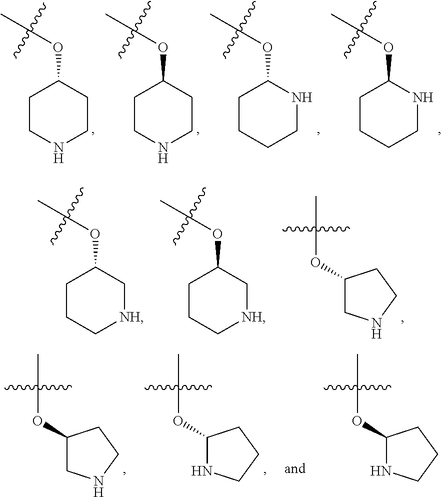

wherein X is an unsubstituted piperidine or pyrrolidine. In another embodiment, L-X is

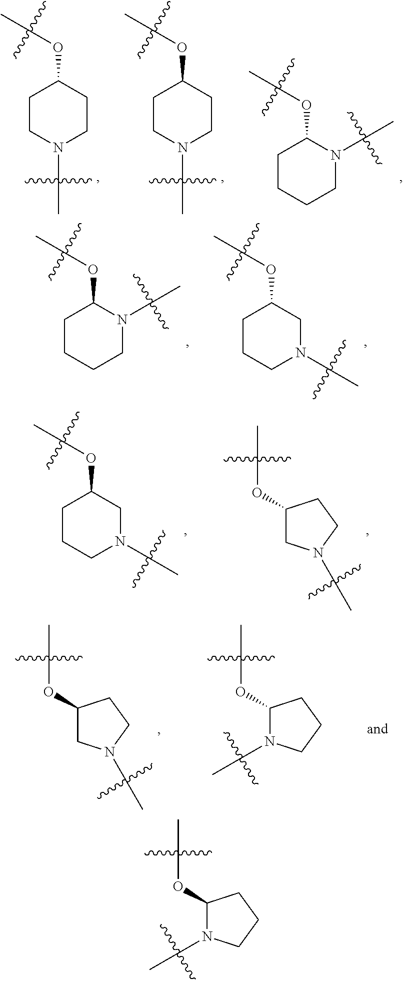

wherein X is a substituted piperidine or pyrrolidine moiety wherein the substitution is at the nitrogen atom.

In an further embodiment, the piperidine or pyrrolidine is substituted at the nitrogen atom with —C(═O)R¹¹ wherein R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl. In another embodiment, the piperidine or pyrrolidine is substituted at the nitrogen atom with C(═O)R¹¹ wherein R¹¹ is substituted or unsubstituted C₁-C₆alkyl. In one embodiment, R¹¹ is methyl, ethyl, n-propyl, iso-propyl, n-butyl, iso-butyl, or tert-butyl. In another embodiment, R₁₁ is methyl. In yet another embodiment, R₁₁ is iso-propyl.

In another embodiment, the piperidine or pyrrolidine is substituted at the nitrogen atom with C(═O)R¹¹ wherein R¹¹ is a substituted or unsubstituted aryl. In one embodiment, the substituted or unsubstituted aryl is a phenyl group. In another embodiment, the substituted or unsubstituted aryl group is a naphthalene group. In yet another embodiment, the piperidine or pyrrolidine is substituted at the nitrogen atom with C(═O)R¹¹ wherein R¹¹ is a phenyl substituted with at least one group selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹², —C(═O)R¹³, —S—R¹³, —S(═O)—R¹³, —S(═O)₂—R¹³, —NR¹²C(═O)—R¹³, —C(═O)N(R¹²)₂, —S(═O)₂N(R¹²)₂, —NR¹²S(═O)₂—R¹³, —OC(═O)N(R¹²)₂, —NR¹²C(═O)O—R¹³, —OC(═O)O—R¹³, —NHC(═O)NH—R¹³, —OC(═O)—R¹³, —N(R¹²)₂, —C₁-C₂alkylN(R¹²)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl, wherein R¹² is hydrogen, C₁-C₆alkyl, phenyl or benzyl and R¹³ is C₁-C₆alkyl, phenyl or benzyl. In another embodiment, R¹¹ is a phenyl substituted with a halogen. In another embodiment, R¹¹ is a phenyl substituted with a substituent selected from —CN, —NO₂ or SH.

In another embodiment, the piperidine or pyrrolidine is substituted at the nitrogen atom with —C(═O)R¹¹ wherein R¹¹ is a substituted or unsubstituted heteroaryl. In one embodiment, the substituted or unsubstituted heteroaryl is pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, 4-azaindolyl, 5-azaindolyl, 6-azaindolyl, 7-azaindolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, imidazo[1,2-a]pyridinyl, thiophenopyridinyl, and furopyridinyl. In another embodiment, the substituted or unsubstituted group is pyridinyl. In yet another embodiment, the piperidine or pyrrolidine is substituted at the nitrogen atom with —C(═O)R¹¹ wherein R¹¹ is a pyridine substituted with at least one group selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹², —C(═O)R¹³, —S—R¹³, —S(═O)—R¹³—S(═O)₂—R¹³, —NR¹²C(═O)—R¹³, —C(═O)N(R¹²)₂, —S(═O)₂N(R¹²)₂, —NR¹²S(═O)₂—R¹³, —OC(═O)N(R¹²)₂, —NR¹²C(═O)O—R¹³, —OC(═O)O—R¹³, —NHC(═O)NH—R¹³, —OC(═O)—R¹³, —N(R¹²)₂, —C₁-C₂alkylN(R¹²)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl, wherein R¹² is hydrogen, C₁-C₆alkyl, phenyl or benzyl and R¹³ is C₁-C₆alkyl, phenyl or benzyl. In another embodiment, R¹¹ is a 2-pyridine, 3-pyridine or 4-pyridine. In yet a further embodiment, the heteroaryl is substituted with a halogen. In another embodiment, R¹¹ is a 2-pyridine, 3-pyridine, or 4-pyridine substituted with a substituent selected from —CN, —NO₂ or SH. In a further embodiment, the heteroaryl is selected from furan, thiophene, benzothiazole, benzoxazole, oxadiazole, or oxazole. In yet a further embodiment, the heteroaryl is furan optionally substituted with a halogen, C₁-C₆alkyl or OH.

In a further embodiment is a compound of Formula (I), (III), (IIIa), (IV), (IVa), (IVb), or (IVc), wherein Y is a substituted or unsubstituted aryl. In one embodiment, Y is a substituted phenyl. In a further embodiment, Y is a phenyl group. In one embodiment, the phenyl is substituted with at least one halogen. In another embodiment, the phenyl is substituted with at least two halogen groups. In a further embodiment, the phenyl group is substituted with a F group. In another embodiment, the phenyl group is substituted with at least one Cl. In another embodiment, with two Cl groups.

In yet another embodiment is a compound of Formula (I), (III), (IIIa), (IV), (IVa), (IVb), or (IVc), wherein Y is a substituted or unsubstituted heteroaryl. In one embodiment the unsubstituted or substituted heteroaryl group is selected from pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, 4-azaindolyl, 5-azaindolyl, 6-azaindolyl, 7-azaindolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, imidazo[1,2-a]pyridinyl, thiophenopyridinyl, and furopyridinyl.

In one embodiment is a compound of Formula (I), (III), (Ma), (IV), (IVa), (IVb), or (IVc), wherein Y is pyridine. In another embodiment, Y is pyrimidine. In a further embodiment, Y is furan. In another embodiment, Y is thiophene. In a further embodiment, Y is indole.

In another embodiment is a compound of Formula (I), (III), (Ma), (IV), (IVa), (IVb), or (IVc), wherein Y is substituted or unsubstituted C₂-C₁₀heterocycloalkyl. In another embodiment, the C₂-C₁₀ heterocycloalkyl is selected from quinolizinyl, dioxinyl, piperidinyl, morpholinyl, thiomorpholinyl, thiazinyl, tetrahydropyridinyl, piperazinyl, oxazinanonyl, dihydropyrrolyl, dihydroimidazolyl, tetrahydrofuranyl, tetrahydropyranyl, dihydrooxazolyl, oxiranyl, pyrrolidinyl, pyrazolidinyl, dihydrothienyl, imidazolidinonyl, pyrrolidinonyl, dihydrofuranonyl, dioxolanonyl, thiazolidinyl, piperidinonyl, indolinyl, tetrahydroquinolinyl, tetrahydroisoquinolinyl, and tetrahydrothienyl.

In one embodiment is a compound of Formula (I), (III), (IIIa), (IV), (IVa), (IVb), or (IVc), wherein Y is substituted or unsubstituted piperazine. In another embodiment, piperazine is substituted with C₁-C₆alkyl. In a further embodiment, C₁-C₆alkyl is selected from methyl, ethyl, n-propyl, isopropyl, n-butyl, iso-butyl, or tert-butyl. In another embodiment, piperazine is substituted with methyl.

Any combination of the groups described above for the various variables is contemplated herein. It is understood that substituents and substitution patterns on the compounds provided herein are selected to provide compounds that are chemically stable and that are synthesized by techniques set forth herein.

Throughout the specification, groups and substituents thereof are chosen to provide stable moieties and compounds.

### Further Forms of Compounds

In some embodiments, compounds described herein possess one or more stereocenters and each center exists in the R or S configuration. The compounds presented herein include all diastereomeric, enantiomeric, and epimeric forms as well as the appropriate mixtures thereof. In some embodiments, separation of steroisomers are performed by chromatography. In other embodiments, individual stereoisomers are obtained by reacting a racemic mixture of the compound with an optically active resolving agent to form a pair of diastereoisomeric compounds, separating the diastereomers and recovering the optically pure enantiomers. In one embodiment the resolution of enantiomers are carried out using covalent diastereomeric derivatives of the compounds described herein, dissociable complexes are also possible (e.g., crystalline diastereomeric salts). Diastereomers have distinct physical properties (e.g., melting points, boiling points, solubilities, reactivity, etc.) and are readily separated by taking advantage of these dissimilarities. In some embodiments, the diastereomers are separated by chiral chromatography, or by separation/resolution techniques based upon differences in solubility. The optically pure enantiomer(s) is/are then recovered, along with the resolving agent, by any practical means that would not result in racemization. A more detailed description of the techniques applicable to the resolution of stereoisomers of compounds from their racemic mixture is found in Jean Jacques, Andre Collet, Samuel H. Wilen, “Enantiomers, Racemates and Resolutions”, John Wiley And Sons, Inc., 1981, herein incorporated by reference for such disclosure. In further embodiments, tereoisomers are obtained by stereoselective synthesis.

In some situations, compounds exist as tautomers. All tautomers are included within the formulas described herein.

The methods and formulations described herein include the use of N-oxides, crystalline forms (also known as polymorphs), or pharmaceutically acceptable salts of compounds described herein, as well as active metabolites of these compounds having the same type of activity. In some situations, compounds exist as tautomers. All tautomers are included within the scope of the compounds presented herein. In addition, the compounds described herein exist in unsolvated as well as solvated forms with pharmaceutically acceptable solvents such as water, ethanol, and the like. The solvated forms of the compounds presented herein are also considered to be disclosed herein.

In some embodiments, the compounds described herein in unoxidized form are prepared from the corresponding N-oxides compounds by treating with a reducing agent, such as, but not limited to, sulfur, sulfur dioxide, triphenyl phosphine, lithium borohydride, sodium borohydride, phosphorus trichloride, phosphorus tribromide, or the like in a suitable inert organic solvent, such as, but not limited to, acetonitrile, ethanol, aqueous dioxane, or the like at 0 to 80° C.

In some embodiments, compounds described herein are prepared as prodrugs. A “prodrug” refers to an agent that is converted into the parent drug in vivo. Prodrugs are often useful because, in some situations, they are easier to administer than the parent drug. In some embodiments, prodrugs are bioavailable by oral administration whereas the parent is not. In other embodiments, the prodrug has improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

To produce a prodrug, a pharmaceutically active compound is modified such that the active compound will be regenerated upon in vivo administration. In some embodiments, the prodrug is designed to alter the metabolic stability or the transport characteristics of a drug, to mask side effects or toxicity, to improve the flavor of a drug or to alter other characteristics or properties of a drug. In some embodiments, once a pharmaceutically active compound is known, knowledge of pharmacodynamic processes and drug metabolism in vivo, aids in the design of prodrugs of the compound. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Saulnier et al., (1994), Bioorganic and Medicinal Chemistry Letters, Vol. 4, p. 1985; Rooseboom et al., Pharmacological Reviews, 56:53-102, 2004; Miller et al., J. Med. Chem. Vol. 46, no. 24, 5097-5116, 2003; Aesop Cho, “Recent Advances in Oral Prodrug Discovery”, Annual Reports in Medicinal Chemistry, Vol. 41, 395-407, 2006).

Prodrug forms of the herein described compounds, wherein the prodrug is metabolized in vivo to produce a derivative as set forth herein are included within the scope of the claims. In some embodiments, some of the herein-described compounds are a prodrug for another derivative or active compound.

In some embodiments prodrugs are easier to administer than the parent drug. In some embodiments the prodrug is bioavailable by oral administration whereas the parent is not. In other embodiments the prodrug has improved solubility in pharmaceutical compositions over the parent drug. In further embodiments, prodrugs are designed as reversible drug derivatives, for use as modifiers to enhance drug transport to site-specific tissues. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure.

Sites on the aromatic ring portion of compounds described herein are susceptible to various metabolic reactions, therefore incorporation of appropriate substituents on the aromatic ring structures, such as, by way of example only, halogens reduces, minimizes or eliminates this metabolic pathway.

In some embodiments the compounds described herein are labeled isotopically (e.g. with a radioisotope) or by other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes incorporated into the present compounds include isotopes of hydrogen, carbon, nitrogen, oxygen, fluorine and chlorine, such as for example, ²H, ³H, ¹³C, ¹⁴C, ¹⁵N, ¹⁸O, ¹⁷O, ³⁵S, ¹⁸F, ³⁶Cl respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, afford certain therapeutic advantages resulting from greater metabolic stability, for example increased in vivo half-life or reduced dosage requirements.

In additional or further embodiments, the compounds described herein are metabolized upon administration to an organism in need to produce a metabolite that is then used to produce a desired effect, including a desired therapeutic effect.

In some embodiments, compounds described herein are formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, nitric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion. In some embodiments, compounds described herein form a coordinate with an organic base, such as, but not limited to, ethanolamine, diethanolamine, triethanolamine, tromethamine, N-methylglucamine, dicyclohexylamine, tris(hydroxymethyl)methylamine. In other embodiments, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. In some embodiments, solvates contain either stoichiometric or non-stoichiometric amounts of a solvent, and form during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein exist in unsolvated as well as solvated forms. In general, the solvated forms are considered equivalent to the unsolvated forms for the purposes of the compounds and methods provided herein.

In some embodiments, compounds described herein are in various forms, including but not limited to, amorphous forms, milled forms and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. In some embodiments, various factors such as the recrystallization solvent, rate of crystallization, and storage temperature cause a single crystal form to dominate.

In other embodiments, the screening and characterization of the pharmaceutically acceptable salts, polymorphs and/or solvates is accomplished by using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UV-VIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

Throughout the specification, groups and substituents thereof are chosen to provide stable moieties and compounds.

### Synthesis of Compounds

The synthesis of compounds described herein are accomplished using means described in the chemical literature, using the methods described herein, or by a combination thereof. In addition, solvents, temperatures and other reaction conditions presented herein vary according to the means described in the chemical literature, using the methods described herein, or by a combination thereof.

The starting materials and reagents used for the synthesis of the compounds described herein are synthesized or are obtained from commercial sources, such as, but not limited to, Sigma-Aldrich, Fluka, Acros Organics, Alfa Aesar, Bachem and the like.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference for such disclosure). General methods for the preparation of compound as disclosed herein are modified by the use of appropriate reagents and conditions, for the introduction of the various moieties found in the formulae as provided herein. As a guide the following synthetic methods are utilized.

Compounds described herein are synthesized starting from compounds that are available from commercial sources or that are prepared using procedures outlined herein.

Using the reaction conditions described herein, cinnamic acid hydroxamate compositions as disclosed herein are obtained in good yields and purity. The compounds prepared by the methods disclosed herein are purified by conventional means such as, filtration, recrystallization, chromatography, distillation, and combinations thereof.

Schemes presented herein are merely illustrative of some methods by which the compounds described herein are synthesized, and various modifications to these schemes are made based on this disclosure.

Formation of Covalent Linkages by Reaction of an Electrophile with a Nucleophile

The compounds described herein are modified using various electrophiles and/or nucleophiles to form new functional groups or substituents. Table A entitled “Examples of Covalent Linkages and Precursors Thereof” lists selected non-limiting examples of covalent linkages and precursor functional groups which yield the covalent linkages. Table A is used as guidance toward the variety of electrophiles and nucleophiles combinations available that provide covalent linkages. Precursor functional groups are shown as electrophilic groups and nucleophilic groups.

TABLE A
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| Hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In the reactions described, it is necessary in certain cases to protect reactive functional groups, for example hydroxy, amino, imino, thio or carboxy groups, where these are desired in the final product, to avoid their unwanted participation in the reactions. Protecting groups are used to block some or all reactive moieties and prevent such groups from participating in chemical reactions until the protective group is removed. In one embodiment, each protective group is removable by a different means. Protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### General Syntheses

### Cinnamic Acid Hydroxyamide Compounds:

Cinnamic acid hydroxyamide compounds described herein are prepared from commercially available materials.

### Synthetic Route I:

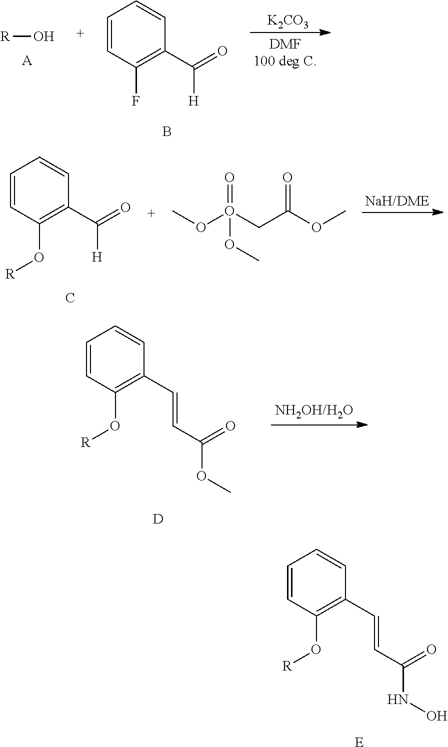

Cinnamic acid hydroxyamide compounds having the structure of compound E can be generally synthesized using Synthetic Route I shown above. Generally, alcohols of compound A (where R is for example, but not limited to, an aryl group, a heteroaryl group or C₂-C₁₀heterocycloalkyl group) are reacted with substituted benzaldehydes of compound B to form compounds having the structure C. Reaction of compounds C with trimethylphosphonoacetate results in compounds having the structure D. Reaction of compound D with a 50% solution of hydroxylamine results in compounds of structure E.

### Synthetic Route II:

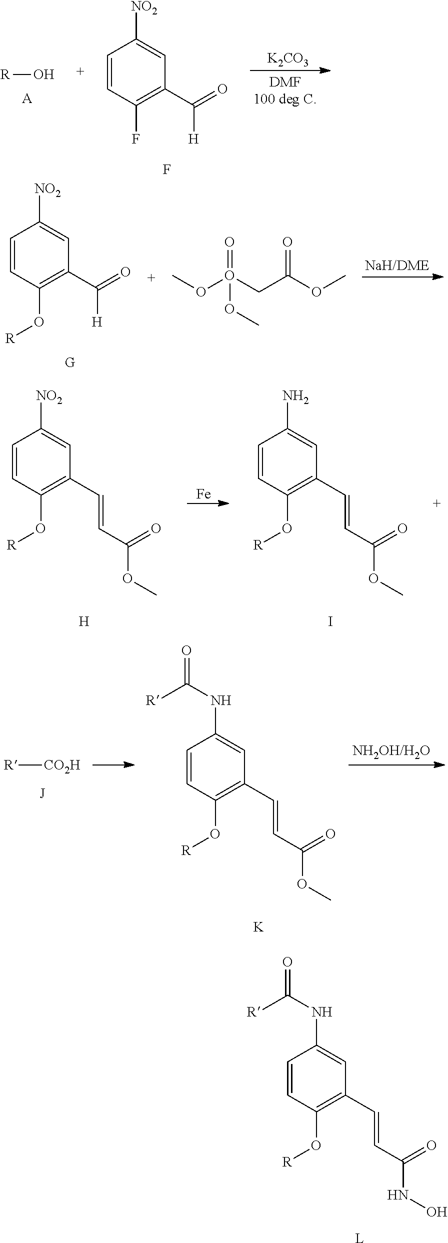

Cinnamic acid hydroxyamide compounds having the structure of compound L can be generally synthesized using Synthetic Route II shown above. Generally, alcohols of compound A (where R is for example, but not limited to, an aryl group, a heteroaryl group or C₂-C₁₀heterocycloalkyl group) are reacted with nitro substituted benzaldehydes of compound F to form compounds having the structure G. Reaction of compounds G with trimethylphosphonoacetate results in compounds having the structure H. Reduction of the nitro group with Fe provides compounds of structure I. Reaction of carboxylic acid compounds J with compounds of structure I provides compounds of structure K. Reaction of compound K with a 50% solution of hydroxylamine results in compounds of structure L.

Throughout the specification, groups and substituents thereof are chosen to provide stable moieties and compounds.

### Certain Terminology

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Definition of standard chemistry terms are found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology are employed. In addition, nucleic acid and amino acid sequences for HDAC8 are disclosed in, e.g., U.S. Pat. No. 6,875,598. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques are used for chemical syntheses, chemical analyses, pharmaceutical preparation, formulation, and delivery, and treatment of patients. Standard techniques are used for recombinant DNA, oligonucleotide synthesis, and tissue culture and transformation (e.g., electroporation, lipofection). Reactions and purification techniques are performed e.g., using kits of manufacturer's specifications or as described herein. The foregoing techniques and procedures are generally performed by conventional methods and as described in various general and more specific references that are cited and discussed throughout the present specification.

It is to be understood that the methods and compositions described herein are not limited to the particular methodology, protocols, cell lines, constructs, and reagents described herein and as such vary. It is also to be understood that the terminology used herein is for the purpose of describing particular embodiments only, and is not intended to limit the scope of the methods, compounds, compositions described herein.

As used herein, C₁-Cₓ includes C₁-C₂, C₁-C₃ . . . C₁-Cₓ. C₁-Cₓ refers to the number of carbon atoms that make up the moiety to which it designates (excluding optional substitutents).

An “alkyl” group refers to an aliphatic hydrocarbon group. In some embodiments, the alkyl moiety is a “saturated alkyl” group, which means that it does not contain any alkene or alkyne moieties. In other embodiments, the alkyl moiety is an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, is branched, straight chain, or cyclic.

The “alkyl” moiety has 1 to 10 carbon atoms (whenever it appears herein, a numerical range such as “1 to 10” refers to each integer in the given range; e.g., “1 to 10 carbon atoms” means that the alkyl group consists of 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein are designated as “C₁-C₆ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, t-butyl, pentyl, iso-pentyl, neo-pentyl, and hexyl. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. In some embodiments alkyl groups are substituted or unsubstituted. Depending on the structure, an alkyl group is either a monoradical or a diradical (i.e., an alkylene group).

An “alkoxy” group refers to a (alkyl)O— group, where alkyl is as defined herein. Examples of alkoxy groups include, but are not limited to, methoxy, ethoxy, propoxy, isopropoxy, buytloxy, cyclopropyloxy, cyclopentyloxy, cyclohexyloxy, and the like.

“Hydroxyalkyl” refers to an alkyl group substituted with hydroxy group(s).

“Hydroxyalkoxy” refers to an alkoxy substituted with hydroxy group(s).

“Hydroxyalkylaminoalkoxy” refers to an alkoxy substituted with an amino group with the amino group substituted with a hydroxyalkyl group as defined herein.

“Alkoxyalkyl” refers to alkyl group substituted with alkoxy group(s).

“Alkoxyalkyloxy” refers to an alkoxy group as defined herein substituted with alkoxy group as defined herein.

“Alkoxycarbonyl” refers to a —C(═O)O-(alkyl) group, where alkyl as defined herein. Non-limiting examples of alkoxycarbonyl groups include, e.g., methoxycarbonyl, ethoxycarbonyl, and the like.

“Alkoxycarbonylamino” refers to a NR(C═O)—O-(alkyl), where alkyl is as defined herein and R is H, alkyl, heteroalkyl, haloalkyl, and the like.

The term “alkenyl” refers to a type of alkyl group in which the first two atoms of the alkyl group form a double bond that is not part of an aromatic group. That is, an alkenyl group begins with the atoms —C(R)═CR₂, wherein R refers to the remaining portions of the alkenyl group, which are the same or different. Non-limiting examples of an alkenyl group include —CH═CH₂, —C(CH₃)═CH₂, —CH═CHCH₃ and —C(CH₃)═CHCH₃. The alkenyl moiety is branched, straight chain, or cyclic (in which case, it would also be known as a “cycloalkenyl” group). Alkenyl groups have 2 to 6 carbons. In some embodiments alkenyl groups are substituted or unsubstituted. Depending on the structure, an alkenyl group is either a monoradical or a diradical (i.e., an alkenylene group).

“Alkenylcarbonyl” refers to a —C(O)-(alkenyl) group, where alkenyl is as defined herein.

“Alkenylcarbonyloxy” refers to a —OC(O)-(alkenyl) group, where alkenyl is as defined herein.

“Alkenyloxy” refers to a —O-(alkenyl) group, where alkenyl is as defined herein.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group. Non-limiting examples of an alkynyl group include —C≡CH, —C≡CCH₃, —C≡CCH₂CH₃ and —C≡CCH₂CH₂CH₃. The “R” portion of the alkynyl moiety is branched, straight chain, or cyclic. In some embodiments an alkynyl group has 2 to 6 carbons. In other embodiments, alkynyl groups are substituted or unsubstituted. Depending on the structure, an alkynyl group is either a monoradical or a diradical (i.e., an alkynylene group).

“Amino” or “amine” refers to a —NH₂ group, an N-oxide derivative, an aliphatic amine or an aromatic amine. Aliphatic amines include: primary amines wherein one of hydrogen atoms is replaced by an organic substituent; secondary amines wherein two of hydrogen atoms are replaced by two organic substituents; and tertiary amines wherein all three substituents on the N atom are organic substituents.

The term “alkylamine” or “alkylamino” refers to the —N(alkyl)ₓH_{y} group, where alkyl is as defined herein and x and y are selected from the group x=1, y=1 and x=2, y=0. When x=2, the alkyl groups, taken together with the nitrogen to which they are attached, optionally form a cyclic ring system. The term “alkylamine” also refers to an amino group substituted with an alkyl group. “Dialkylamino” refers to a N(alkyl)₂ group, where alkyl is as defined herein.

“Aminoalkyl” refers to an alkyl group as is defined herein that is substituted with an amino group.

“Aminoalkoxy” refers to an alkoxy group substituted with an amino group.

“Aminocarbonyl” refers to a —CONH₂ group.

“Aminosulfonyl” means an —S(O)₂NH₂ radical.

The term “alkylaminoalkyl” refers to an alkyl group, as is defined herein, substituted with an alkylamine as is defined herein. “Dialkylaminoalkyl” refers to an alkyl group that is substituted with a dialkylamino group.

“Alkylaminoalkoxy” refers to a alkoxy substituted with an alkylamine.

“Alkylaminocarbonyl” means a —C(O)R radical where R is alkylamino as defined herein.

“Alkylaminocarbonylamino” refers to —NHC(═O)-(alkylamino).

“Alkylaminocarbonyloxy” refers to —OC(═O)-(alkylamino).

“Alkylaminosulfonyl” refers to —S(═O)₂NHR radical where R is alkyl, as defined herein.

“Alkylcarbonyl” means a —C(═O)R radical where R is alkyl as defined herein.

“Alkylcarbonylamino” means a —NR′C(═O)-(alkyl), where R′ is hydrogen, alkyl, haloalkyl, heteroalkyl.

“Alkylcarbonyloxy” means a —OC(═O)R radical where R is alkyl as defined herein.

“Dialkylaminoalkyloxy” refers to a alkoxy substituted with a dialkylamino.

“Dialkylaminocarbonyl” refers to —C(═O)R, where R is dialkylamino.

“Dialkylaminocarbonylamino” refers to —NR′—C(═O)-(dialkylamino), where R′ is hydrogen, alkyl, heteroalkyl, haloalkyl, and dialkylaminocarbonyl as defined herein.

“Dialkylaminocarbonyloxy” means an —O(C═O)-(dialkylamino), dialkylaminocarbonyl as defined herein.

“Dialkylaminosulfonyl” refers to —S(O)₂NR₂, where R is alkyl as defined herein.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). In some embodiments, rings are optionally substituted. In other embodiments rings are monocyclic or polycyclic.

The term “membered ring” refers to any cyclic structure. The term “membered” is meant to denote the number of skeletal atoms that constitute the ring. Thus, for example, cyclohexyl, phenyl, pyridine, piperidine, morpholine, piperazine, pyridazine, pyrimidine, pyrazine, pyran and thiopyran are 6-membered rings; and cyclopentyl, pyrrolidine, imidazole, oxazole, thiazole, pyrrole, furan, and thiophene are 5-membered rings.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e a heteroatom). Heterocycle includes heteroaryl and heterocycloalkyl. In some embodiments carbocycles and heterocycles are optionally substituted.

The term “aromatic” refers to a planar ring having a delocalized π-electron system containing 4n+2π electrons, where n is an integer. In some embodiments aromatic rings are formed from five, six, seven, eight, nine, or more than nine atoms. In other embodiments aromatics are optionally substituted. The term “aromatic” includes both carbocyclic aryl (“aryl”, e.g., phenyl) and heterocyclic aryl (or “heteroaryl” or “hetero aromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. In some embodiments, aryl rings are formed by five, six, seven, eight, nine, ten or more than ten carbon atoms. In some embodiments, aryl groups are optionally substituted. In some embodiments, an aryl is a C₆-C₁₀aryl. Examples of aryl groups include, but are not limited to phenyl, and naphthalenyl. In one aspect, an aryl is a phenyl. Depending on the structure, an aryl group is either a monoradical or a diradical (i.e., an arylene group).

“Aralkyl” or “arylalkyl” refers to an alkyl group as is defined herein substituted with an aryl group as is defined herein.

“Phenylalkyl” refers to an alkyl substituted with a phenyl.

The term “cycloalkyl” refers to a monocyclic or polycyclic non-aromatic radical, wherein each of the atoms forming the ring (i.e. skeletal atoms) is a carbon atom. Cycloalkyls are saturated, or partially unsaturated. In some embodiments. cycloalkyls are fused with an aromatic ring. Cycloalkyl groups include groups having from 3 to 10 ring atoms. Illustrative examples of cycloalkyl groups include, but are not limited to, the following:

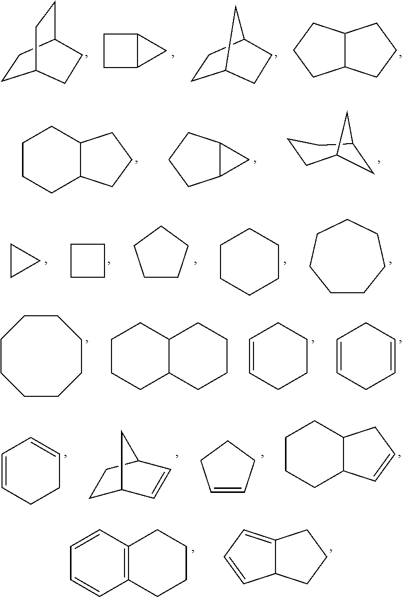

and the like. Cycloalkyls include, but are not limited to, cyclopropyl, cyclobutyl, cyclopentyl, cyclohexyl, cycloheptyl, and cyclooctyl. In one aspect, a cycloalkyl is a C₃-C₆cycloalkyl.

“Cycloalkylalkyl” refers to an alkyl, as is defined herein, substituted with a cycloalkyl, as is defined herein.

“Cycloalkylcarbonyl” refers to —C(═O)-cycloalkyl.

The term “heterocycle” refers to heteroaromatic and heteroalicyclic groups containing one to four ring heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are C-attached or N-attached where such is possible. For example, a group derived from pyrrole is named pyrrol-1-yl (N-attached) or pyrrol-3-yl (C-attached). Further, a group derived from imidazole is named imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. Polycyclic heteroaryl groups are fused or non-fused. Illustrative examples of heteroaryl groups include the following moieties:

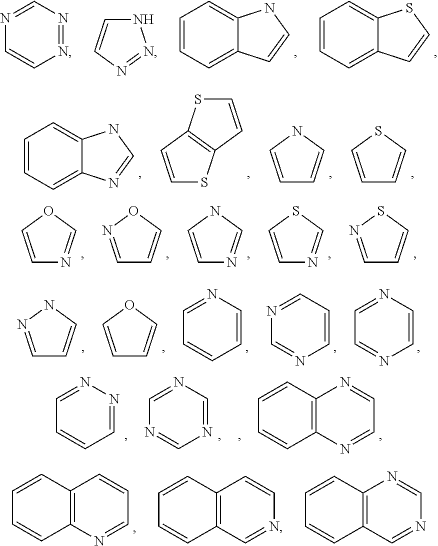

and the like. In one aspect, a heteroaryl includes 0-3 N atoms. In one aspect, a heteroaryl includes 1-3 N atoms. In one aspect, a heteroaryl includes 0-3 N atoms, 0-1 O atoms, and 0-1 S atoms. In one aspect, a heteroaryl is a monocyclic or bicyclic heteroaryl. In one aspect, a heteroaryl is a monocyclic heteroaryl. In one aspect, the heteroaryl is a C₁-C₁₀heteroaryl. In another aspect, the heteroaryl is a C₂-C₉heteroaryl. In one aspect, monocyclic heteroaryl is a C₁-C₅heteroaryl. In one aspect, bicyclic heteroaryl is a C₅-C₁₀heteroaryl. Depending on the structure, a heteroaryl group can be a monoradical or a diradical (i.e., a heteroarylene group).

In some embodiments, substituted or unsubstituted heteroaryl groups are selected from among pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, 4-azaindolyl, 5-azaindolyl, 6-azaindolyl, 7-azaindolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, imidazo[1,2-a]pyridinyl, thiophenopyridinyl, and furopyridinyl. In other embodiments, substituted or unsubstituted heteroaryl groups are selected from among pyridinyl, pyrimidinyl, pyrazinyl, quinolinyl, isoquinolinyl, indolyl, benzimidazolyl, benzofuranyl, cinnolinyl, indazolyl, indolizinyl, phthalazinyl, pyridazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, naphthyridinyl, imidazo[1,2-a]pyridinyl, thiophenopyridinyl, and furopyridinyl. In yet other embodiments, substituted or unsubstituted heteroaryl groups are selected from among pyridinyl, pyrimidinyl, pyrazinyl, quinolinyl, isoquinolinyl, pyridazinyl, quinazolinyl, quinoxalinyl. In still other embodiments, substituted or unsubstituted heteroaryl groups are selected from among pyridinyl, and quinolinyl.

“Heteroaralkyl” or “heteroarylalkyl” refers to an alkyl, as is defined herein, substituted with a heteroaryl as is defined herein.

A “heteroalicyclic” group or “heterocycloalkyl” group refers to a cycloalkyl group, wherein at least one skeletal ring atom is a heteroatom selected from nitrogen, oxygen and sulfur. The radicals are fused with an aryl or heteroaryl. Illustrative examples of heterocycloalkyl groups, also referred to as non-aromatic heterocycles, include:

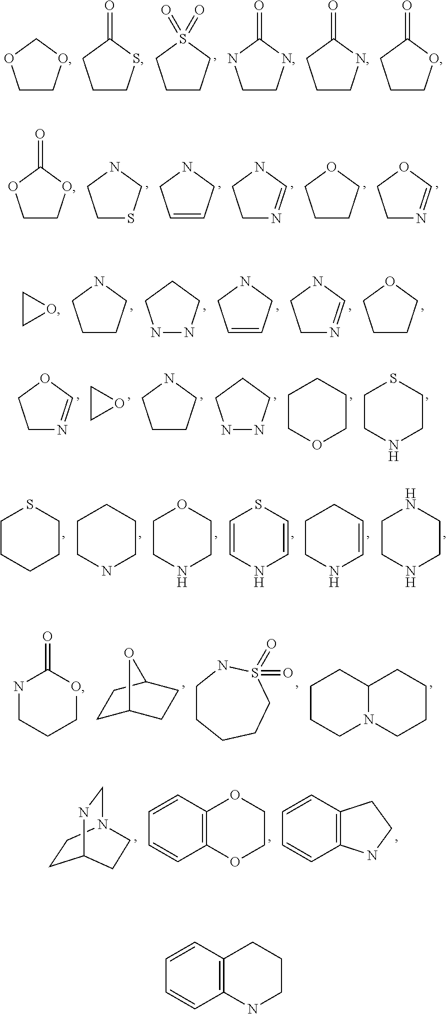

and the like. The term heteroalicyclic also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteratoms) that make up the heterocycloalkyl (i.e skeletal atoms of the heterocycloalkyl ring). In one aspect, a heterocycloalkyl is a C₂-C₁₀heterocycloalkyl. In another aspect, a heterocycloalkyl is a C₄-C₁₀heterocycloalkyl.

In some embodiments, substituted or unsubstituted heterocycloalkyl groups are selected from among quinolizinyl, dioxinyl, piperidinyl, morpholinyl, thiomorpholinyl, thiazinyl, tetrahydropyridinyl, piperazinyl, oxazinanonyl, dihydropyrrolyl, dihydroimidazolyl, tetrahydrofuranyl, tetrahydropyranyl, dihydrooxazolyl, oxiranyl, pyrrolidinyl, pyrazolidinyl, dihydrothienyl, imidazolidinonyl, pyrrolidinonyl, dihydrofuranonyl, dioxolanonyl, thiazolidinyl, piperidinonyl, indolinyl, tetrahydroquinolinyl, tetrahydroisoquinolinyl, and tetrahydrothienyl. In other embodiments, substituted or unsubstituted heterocycloalkyl groups are selected from among piperidinyl, morpholinyl, piperazinyl, dihydropyrrolyl, dihydroimidazolyl, tetrahydrofuranyl, dihydrooxazolyl, pyrrolidinyl, pyrazolidinyl, dihydrothienyl, imidazolidinonyl, pyrrolidinonyl, piperidinonyl, indolinyl, tetrahydroquinolinyl, tetrahydroisoquinolinyl, and tetrahydrothienyl. In yet other embodiments, substituted or unsubstituted heterocycloalkyl groups are selected from among piperidinyl, morpholinyl, piperazinyl, tetrahydrofuranyl, pyrrolidinyl, pyrrolidinonyl, piperidinonyl, indolinyl, tetrahydroquinolinyl, and tetrahydrothienyl. In some embodiments, substituted or unsubstituted heterocycloalkyl groups are selected from among piperidinyl, morpholinyl, thiomorpholinyl, piperazinyl, and pyrrolidinyl.

“Heterocycloalkylalkyl” refers to an alkyl, as defined herein, substituted with a heterocycloalkyl, as defined herein.

“Heterocycloalkylalkoxy” refers to an alkoxy, as defined herein, substituted with a heterocycloalkyl, as defined herein wherein heterocycloalkyl includes alkyl substituents.

The term “hydroxamate”, “hydroxamic acid”, “N-hydroxycarboxamide” or “carboxylic acid hydroxyamide” refers to:

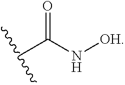 [SMILES: *C(=O)NO]

The term “halo” or, alternatively, “halogen” means fluoro, chloro, bromo and iodo.

The terms “haloalkyl,” “haloalkenyl,” “haloalkynyl” and “haloalkoxy” include alkyl, alkenyl, alkynyl and alkoxy structures that are substituted with one or more halogens. In some embodiments, the halogens are the same or are different. The terms “fluoroalkyl” and “fluoroalkoxy” include haloalkyl and haloalkoxy groups, respectively, in which the halo is fluorine. Non-limiting examples of haloalkyls include —CH₂Cl, —CF₃, —CHF₂, —CH₂CF₃, —CF₂CF₃, —CF(CH₃)₃, and the like. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of haloalkoxy groups include —OCF₃, —OCHF₂, —OCH₂F, —OCH₂CF₃, —OCF₂CF₃, —OCF₂CF₂CF₃, —OCF(CH₃)₃, and the like.

The terms “heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus, silicon, or combinations thereof. The heteroatom(s) are placed at any position of the heteroalkyl group. In some embodiments, up to two heteroatoms are consecutive, such as, by way of example, —CH₂—NH—OCH₃ and CH₂—O—Si(CH₃)₃. Excluding the number of heteroatoms, a “heteroalkyl” includes from 1 to 6 carbon atoms, a “heteroalkenyl” includes from 2 to 6 carbons atoms, and a “heteroalkynyl” includes from 2 to 6 carbon atoms.

The term “moiety” refers to a specific segment or functional group of a molecule. Chemical moieties are often recognized chemical entities embedded in or appended to a molecule.

“Cyanoalkylaminocarbonyl” refers to a —C(═O)NR′(cyanoalkyl) group, where R′ is hydrogen, alkyl, heteroalkyl, haloalkyl, as is defined herein, cyanoalkyl is as defined herein.

An “isothiocyanato” group refers to a —NCS group.

“Alkylthio” means an —SR radical where R is alkyl as defined herein.

“Acylamino” refers to a RC(═O)N(R′)— group, where R′ is hydrogen, hydroxy, alkyl, or alkoxy. In some embodiments, R′ is H or R.

“Alkylsulfonyl” means an —S(O)R radical where R is alkyl as defined herein.

“Alkylsulfonyl” means a —SO₂R radical where R is alkyl as defined herein.

“Alkylsulfonylamino” means a N(R)SO₂R group, where R′ is hydrogen, alkyl, heteroalkyl, haloalkyl, as is defined herein, and R is alkyl as is defined herein.

“Phenylsulfonyl” refers to means a —S(═O)₂-phenyl moiety.

“Phenylsulfonylamino” refers to a NR′SO₂— (phenyl) where R′ is hydrogen, alkyl, heteroalkyl, haloalkyl, as is defined herein.

“Heteroarylaminocarbonyl” refers to a —C(═O)NR′ (heteroaryl) group, where R′ is hydrogen, alkyl, heteroalkyl, haloalkyl, as is defined herein, and heteroaryl is as defined herein.

“Arylaminocarbonyl” refers to a —C(═O)NR′ (aryl) group, where R′ is hydrogen, alkyl, heteroalkyl, haloalkyl, as is defined herein, and aryl is as defined herein.

“Arylcarbonylamino” refers to —NR′C(═O)-(aryl) group, where R′ is hydrogen, alkyl, heteroalkyl, haloalkyl, as is defined herein, and aryl is as defined herein.

As used herein, the substituent “R” appearing by itself and without a number designation refers to a substituent selected from among from alkyl, haloalkyl, heteroalkyl, alkenyl, cycloalkyl, cycloalkylalkyl, aryl, arylalkyl, heteroaryl (bonded through a ring carbon), heteroarylalkyl, heterocycloalkyl, and heterocycloalkylalkyl.

The term “optionally substituted” or “substituted” means that the referenced group is substituted with one or more additional group(s) individually and independently selected from alkyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, cyano, halo, acyl, acyloxy, isocyanato, thiocyanato, isothiocyanato, nitro, haloalkyl, fluoroalkyl, and amino, including mono- and di-substituted amino groups (e.g. NH₂, —NHR, —N(R)₂), and the protected derivatives thereof. By way of example, an optional substituent is L^{s}R^{s}, wherein each L^{s} is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, —(C₁-C₆alkyl)-, or —(C₂-C₆alkenyl)-; and each R^{s} is independently selected from among H, (C₁-C₆alkyl), (C₃-C₈cycloalkyl), aryl, heteroaryl, heteocycloalkyl, and C₁-C₆heteroalkyl. In one aspect, substituted groups are substituted with one or more substituents selected from halogen, —OH, —OC₁-C₄alkyl, C₁-C₄alkyl, C₁-C₄heteroalkyl, C₁-C₄fluoroalkyl and —OC₁-C₄fluoroalkyl. In yet other aspect, substituted groups are substituted with one or more substituents selected from F, Cl, Br, —OH, —OCH₃, —CH₃, and —CF₃. In yet other embodiments, substituted groups are substituted with one or more substituents selected from F, Cl, and Br. In one aspect, substituted groups are substituted with one of the preceding groups. The protecting groups that form the protective derivatives of the above substituents are found in references such as Greene and Wuts, above.

The compounds presented herein possess one or more stereocenters and each center exists in the R or S configuration. The compounds presented herein include all diastereomeric, enantiomeric, and epimeric forms as well as the appropriate mixtures thereof. Stereoisomers are obtained, if desired, by separation of stereoisomers by chiral chromatographic columns.

The methods and formulations described herein include the use of N-oxides, crystalline forms (also known as polymorphs), or pharmaceutically acceptable salts of compounds having the structure of Formula I, as well as active metabolites of these compounds having the same type of activity. In some situations, compounds exist as tautomers. All tautomers are included within the scope of the compounds presented herein. In addition, the compounds described herein exist in unsolvated as well as solvated forms with pharmaceutically acceptable solvents such as water, ethanol, and the like. The solvated forms of the compounds presented herein are also considered to be disclosed herein.

The terms “kit” and “article of manufacture” are used as synonyms.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

A “selective HDAC8 inhibitor,” as used herein, refers to a compound that has an IC₅₀ for inhibition of HDAC8 deacetylase activity that is at least about 5 fold to more than about 500 fold lower than the IC₅₀ for inhibition of deacetylase activity of another HDAC. In some embodiments, the selective HDAC8 inhibitor has an IC₅₀ for inhibition of HDAC8 deacetylase activity that is about 5, about 10, about 50, about 100, about 150, about 200, about 250, about 300, about 350, about 400, about 450 or more than about 500 fold lower than the IC₅₀ for inhibition of deacetylase activity of another HDAC. In one embodiment, the selective HDAC8 inhibitor has an IC₅₀ for inhibition of HDAC8 deacetylase activity that is at least about 10 fold lower than the IC₅₀ for inhibition of deacetylase activity of at least one of HDAC1, HDAC2, HDAC3, HDAC6, HDAC10, and HDAC11; in another embodiment at least two of HDAC1, HDAC2, HDAC3, HDAC6, HDAC10, and HDAC11; in another embodiment all of HDAC1, HDAC2, HDAC3, HDAC6, HDAC10, and HDAC11. In another embodiment, the selective HDAC8 inhibitor has an IC₅₀ for HDAC8 deacetylase activity that is at least about 20 fold lower than the IC₅₀ for inhibition of deacetylase activity of at least one of HDAC1, HDAC2, HDAC3, HDAC6, HDAC10, and HDAC11; in another embodiment at least two of HDAC1, HDAC2, HDAC3, HDAC6, HDAC10, and HDAC11; in another embodiment all of HDAC1, HDAC2, HDAC3, HDAC6, HDAC10, and HDAC11.

As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that is attributed to or associated with administration of the compound or composition.

The terms “inhibits”, “inhibiting”, or “inhibitor” of HDAC, as used herein, refer to inhibition of histone deacetylase activity.

The term “acceptable” with respect to a formulation, composition or ingredient, as used herein, means having no persistent detrimental effect on the general health of the subject being treated.

By “pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutical composition” refers to a mixture of the compound described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound include but are not limited to: intravenous, oral, aerosol, parenteral, ophthalmic, pulmonary and topical administration.

The terms “effective amount” or “therapeutically effective amount,” as used herein, refer to a sufficient amount of an agent or a compound being administered which will relieve to some extent one or more of the symptoms of the disease or condition being treated. The result is reduction and/or alleviation of the signs, symptoms, or causes of a disease, or any other desired alteration of a biological system. For example, an “effective amount” for therapeutic uses is the amount of the composition comprising a HDAC8 inhibiting compound as disclosed herein required to provide a clinically significant decrease in disease symptoms. An appropriate “effective” amount in any individual case is determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “co-administration” or the like, as used herein, are meant to encompass administration of the selected therapeutic agents to a single patient, and are intended to include treatment regimens in which the agents are administered by the same or different route of administration or at the same or different time.

The term “carrier,” as used herein, refers to relatively nontoxic chemical compounds or agents that facilitate the incorporation of a compound into cells or tissues.

The term “diluent” refers to chemical compounds that are used to dilute the compound of interest prior to delivery. Diluents are also used to stabilize compounds because they provide a more stable environment. Salts dissolved in buffered solutions (which also provide pH control or maintenance) are utilized, including, but not limited to a phosphate buffered saline solution.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism is obtained from The Pharmacological Basis of Therapeutics, 9th Edition, McGraw-Hill (1996). Metabolites of the compounds disclosed herein are identified either by administration of compounds to a host and analysis of tissue samples from the host, or by incubation of compounds with hepatic cells in vitro and analysis of the resulting compounds.

“Bioavailability” refers to the percentage of the weight of compounds disclosed herein, that is delivered into the general circulation of the animal or human being studied. The total exposure (AUC(0-∞)) of a drug when administered intravenously is usually defined as 100% bioavailable (F %). “Oral bioavailability” refers to the extent to which the compounds disclosed herein, are absorbed into the general circulation when the pharmaceutical composition is taken orally as compared to intravenous injection.

“Plasma concentration” refers to the concentration of the compounds disclosed herein, in the plasma component of blood of a subject. It is understood that the plasma concentration of the compounds described herein vary significantly between subjects, due to variability with respect to metabolism and/or possible interactions with other therapeutic agents. In accordance with one embodiment disclosed herein, the blood plasma concentration of the compounds disclosed herein vary from subject to subject. Likewise, values such as maximum plasma concentration (Cmax) or time to reach maximum plasma concentration (Tmax), or total area under the plasma concentration time curve (AUC(0-∞)) varies from subject to subject. Due to this variability, the amount necessary to constitute “a therapeutically effective amount” of a compound varies from subject to subject.

“Pharmacokinetics” refers to the factors which determine the attainment and maintenance of the appropriate concentration of drug at a site of action.

### Examples of Pharmaceutical Compositions and Methods of Administration

Suitable routes of administration include, but are not limited to, oral, intravenous, rectal, aerosol, parenteral, ophthalmic, pulmonary, transmucosal, transdermal, vaginal, otic, nasal, intramuscular injection, subcutaneous injection, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In certain embodiments, a compound as described herein is administered in a local rather than systemic manner. In other embodiments, the compound as described herein is provided in the form of a rapid release formulation, in the form of an extended release formulation, or in the form of an intermediate release formulation. In yet other embodiments, the compound described herein is administered topically.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

A pharmaceutical composition refers to a mixture of a HDAC8 inhibitor compound described herein with other chemical components, such as carriers, stabilizers, diluents, dispersing agents, suspending agents, thickening agents, and/or excipients. In certain embodiments, the pharmaceutical composition facilitates administration of the compound to a mammal.

In one embodiment, HDAC8 inhibitor compounds described herein are formulated in an aqueous solution. In specific embodiments, the aqueous solution is selected from, by way of example only, a physiologically compatible buffer, such as Hank's solution, Ringer's solution, or physiological saline buffer. In other embodiments, HDAC8 inhibitor compounds described herein are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or nonaqueous solutions.

In another embodiment, compounds described herein are formulated for oral administration. The compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or pills. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

Oral dosage forms also include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the HDAC8 inhibitor compounds described herein are administered topically. Topically administrable compositions include solutions, suspensions, lotions, gels, pastes, medicated sticks, balms, creams or ointments.

In other embodiments, the HDAC8 inhibitor compounds described herein are formulated for administration by inhalation. Various forms suitable for administration by inhalation include, but are not limited to, aerosols, mists or powders.

The active ingredient in the pharmaceutical compositions is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions comprising the compounds described herein are administered to a patient susceptible to or otherwise at risk of a particular disease, disorder or condition. In this use, the precise amounts also depend on the patient's state of health, weight, and the like.

In some embodiments pharmaceutical compositions are formulated in a conventional manner using one or more physiologically acceptable carriers including excipients and auxiliaries which facilitate processing of the active compounds into preparations which are used pharmaceutically. Proper formulation is dependent upon the route of administration chosen.

### Examples of Methods of Dosing and Treatment Regimens

The compounds described herein are used in the preparation of medicaments for the inhibition of HDAC8, or for the treatment of diseases or conditions that would benefit, at least in part, from inhibition of HDAC8. In addition, a method for treating any of the diseases or conditions described herein in a subject in need of such treatment, involves administration of pharmaceutical compositions containing at least one compound described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

The compositions containing the compound(s) described herein are administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. One determines such therapeutically effective amounts by, e.g., a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. One determines such prophylactically effective amounts by e.g., a dose escalation clinical trial. When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds are given continuously; alternatively, the dose of drug being administered is temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. In some embodiments, the dose reduction during a drug holiday is from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. Some patients require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but will be determined according to the particular circumstances surrounding the case, including, e.g., the specific agent being administered, the route of administration, the condition being treated, and the subject or host being treated. In general, however, doses employed for adult human treatment will typically be in the range of about 0.02 to about 5000 mg per day, in other embodiments about 1 to about 1500 mg per day. In some embodiments the desired dose is presented in a single dose or as divided doses administered simultaneously (or over a short period of time) or at appropriate intervals, for example as two, three, four or more sub-doses per day.

The pharmaceutical composition described herein is in unit dosage forms suitable for single administration of precise dosages. In unit dosage form, the formulation is divided into unit doses containing appropriate quantities of one or more compound. The unit dosage is in the form of a package containing discrete quantities of the formulation. Non-limiting examples are packaged tablets or capsules, and powders in vials or ampoules. Aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi-dose containers, with an added preservative.

The daily dosages appropriate for the compounds described herein described herein are from about 0.01 to about 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to about 50 mg active ingredient. The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are contemplated herein. The data obtained from cell culture assays and animal studies is used in formulating a range of dosage for use in human. In some embodiments, the dosage of such compounds lies within a range of circulating concentrations that include the ED50 with minimal toxicity. The dosage varies within this range depending upon the dosage form employed and the route of administration utilized.

### Combination Treatments

The compounds and compositions described herein are also used in combination with other therapeutic agents that are selected for their therapeutic value for the condition to be treated. In general, the compositions described herein and, in embodiments where combinational therapy is employed, other agents are not administered in the same pharmaceutical composition, and are administered by different routes because of different physical and chemical characteristics. The initial administration is made according to established protocols and based upon the observed effects, the dosage, modes of administration and times of administration.

In certain instances, it is appropriate to administer at least one compound described herein in combination with another therapeutic agent. By way of example only, if one of the side effects experienced by a patient upon receiving one of the compounds herein, such as a hydroxamic acid compound described herein, is nausea, then it is appropriate to administer an anti-nausea agent in combination with the initial therapeutic agent. Or, by way of example only, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by a patient is increased by administering one of the compounds described herein with another therapeutic agent (which also includes a therapeutic regimen) that also has therapeutic benefit. In any case, regardless of the disease, disorder or condition being treated, the overall benefit experienced by the patient is additive of the two therapeutic agents or the patient experiences a synergistic benefit.

The particular choice of compounds used will depend upon the diagnosis of the attending physicians and their judgment of the condition of the patient and the appropriate treatment protocol. The compounds are administered concurrently (e.g., simultaneously, essentially simultaneously or within the same treatment protocol) or sequentially, depending upon the nature of the disease, disorder, or condition, the condition of the patient, and the actual choice of compounds used. The determination of the order of administration, and the number of repetitions of administration of each therapeutic agent during a treatment protocol, is determined after evaluation of the disease being treated and the condition of the patient.

For combination therapies described herein, dosages of the co-administered compounds will vary depending on the type of co-drug employed, on the specific drug employed, on the disease or condition being treated and so forth. In addition, when co-administered with one or more biologically active agents, the compound provided herein is administered either simultaneously with the biologically active agent(s), or sequentially. If administered sequentially, the attending physician will decide on the appropriate sequence of administering protein in combination with the biologically active agent(s).

In any case, the multiple therapeutic agents (one of which is a HDAC8 selective compound described herein) are administered in any order or even simultaneously. If simultaneously, the multiple therapeutic agents are provided in a single, unified form, or in multiple forms (by way of example only, either as a single pill or as two separate pills). In some embodiments the therapeutic agents are given in multiple doses, or both are given as multiple doses. If not simultaneous, the timing between the multiple doses varies from more than zero weeks to less than four weeks. In addition, the combination methods, compositions and formulations are not to be limited to the use of only two agents; the use of multiple therapeutic combinations is also envisioned.

It is understood that the dosage regimen to treat, prevent, or ameliorate the condition(s) for which relief is sought, is modified in accordance with a variety of factors. These factors include the disorder or condition from which the subject suffers, as well as the age, weight, sex, diet, and medical condition of the subject. Thus, the dosage regimen actually employed varies widely and therefore deviates from the dosage regimens set forth herein.

The pharmaceutical agents which make up the combination therapy disclosed herein are a combined dosage form or in separate dosage forms intended for substantially simultaneous administration. The pharmaceutical agents that make up the combination therapy are administered sequentially, with either therapeutic compound being administered by a regimen calling for two-step administration. The two-step administration regimen calls for sequential administration of the active agents or spaced-apart administration of the separate active agents. The time period between the multiple administration steps ranges from, a few minutes to several hours, depending upon the properties of each pharmaceutical agent, such as potency, solubility, bioavailability, plasma half-life and kinetic profile of the pharmaceutical agent. Circadian variation of the target molecule concentration also determines the optimal dose interval.

In addition, the compounds described herein are used in combination with procedures that provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a mutant gene that is known to be correlated with certain diseases or conditions.

The compounds described herein and combination therapies are administered before, during or after the occurrence of a disease or condition, and the timing of administering the composition containing a compound varies. Thus, for example, the compounds are used as a prophylactic and are administered continuously to subjects with a propensity to develop conditions or diseases in order to prevent the occurrence of the disease or condition. The compounds and compositions are administered to a subject during or as soon as possible after the onset of the symptoms. The administration of the compounds are initiated within the first 48 hours of the onset of the symptoms, in other embodiments, within the first 48 hours of the onset of the symptoms, in further embodiments, within the first 6 hours of the onset of the symptoms, and in yet further embodiments within 3 hours of the onset of the symptoms. The initial administration is via any route practical, such as, for example, an intravenous injection, a bolus injection, infusion over 5 minutes to about 5 hours, a pill, a capsule, transdermal patch, buccal delivery, and the like, or combination thereof. In some embodiments, a compound is administered as soon as is practicable after the onset of a disease or condition is detected or suspected, and for a length of time necessary for the treatment of the disease, such as, for example, from about 1 month to about 3 months. The length of treatment varies for each subject, and the length is determined using the known criteria. For example, the compound or a formulation containing the compound is administered for at least 2 weeks, in some embodiments, about 1 month to about 5 years, and in other embodiments from about 1 month to about 3 years.

### Anti-Cancer Agents

Combinations of selective HDAC8 inhibitors described herein with other anti-cancer or chemotherapeutic agents are described herein. Examples of such anti-cancer or chemotherapeutic agents are found in Cancer Principles and Practice of Oncology by V. T. Devita and S. Hellman (editors), 6^{th} edition (Feb. 15, 2001), Lippincott Williams & Wilkins Publishers. Combinations of agents are determined based on the particular characteristics of the drugs and the cancer involved.

The term “combination” as used herein, means a product that results from the mixing or combining of more than one active ingredient and includes both fixed and non-fixed combinations of the active ingredients. In some embodiments, the combination is a fixed combination. The term “fixed combination” means that the active ingredients, e.g. a cinnamic hydroxyamide compound described herein, and a co-agent, are both administered to a patient simultaneously in the form of a single entity or dosage. In some embodiments, the combination is a non-fixed combination. The term “non-fixed combination” means that the active ingredients, e.g. a compound described herein, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

In one aspect, HDAC inhibitors disclosed herein are administered in combination with an agent selected from anthrocyclins, fludarabine, flavopiridol, imatinib, bortezomib, anti-angiogenesis agents and nuclear receptor ligands, such as, all-trans retinoic acid and tumor necrosis factor-related apoptosis-inducing ligand.

Anti-cancer agents and/or agents used in chemotherapy include, but are not limited to, the following: estrogen receptor modulators, androgen receptor modulators, retinoid receptor modulators, cytotoxic/cytostatic agents, antiproliferative agents, prenyl-protein transferase inhibitors, nitrogen mustards, nitroso ureas, angiogenesis inhibitors, inhibitors of cell proliferation and survival signaling pathway, apoptosis inducing agents, agents that interfere with cell cycle checkpoints, agents that interfere with receptor tyrosine kinases (RTKs), integrin blockers, NSAIDs, inhibitors of inherent multidrug resistance (MDR), anti-emetic agents, agents useful in the treatment of anemia, agents useful in the treatment of neutropenia, immunologic-enhancing drugs, biphosphonates, aromatase inhibitors, agents inducing terminal differencation of neoplastic cells, γ-secretase inhibitors, cancer vaccines, and any combination thereof.

Where the subject is suffering from a cancer (e.g., a T-cell lymphoma), a selective HDAC8 inhibitor is used in any combination with one or more other anti-cancer agents. Examples of anti-cancer agents include, but are not limited to, any of the following: 5-aza-2′-deoxycytidine, all trans retinoic acid, doxorubicin, vincristine, etoposide, gemcitabine, imatinib, 17-N-allylamino-17-demethoxygeldanamycin (17-AAG), flavopiridol, LY294002, bortezomib, trastuzumab, BAY 11-7082, PKC412, or PD184352.

Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and are useful for treating cancer in combination with the compounds described herein.

Other anti-cancer agents that are employed in combination with a selective HDAC8 inhibitor include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine hydrochloride; hydroxyurea; idarubicin hydrochloride; ifosfamide; iimofosine; interleukin I1 (including recombinant interleukin II, or rlL2), interferon alfa-2a; interferon alfa-2b; interferon alfa-nl; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that are employed in combination with a selective HDAC8 inhibitor include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that are employed in combination with a selective HDAC8 inhibitor include alkylating agents, antimetabolites, natural products, or hormones, nitrogen mustards (e.g., mechloroethamine, cyclophosphamide, chlorambucil, etc.), alkyl sulfonates (e.g., busulfan), nitrosoureas (e.g., carmustine, lomusitne, ete.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products useful in combination with a selective HDAC8 inhibitor include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that are employed in combination a selective HDAC8 inhibitor include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, ete.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

Examples of hormones and antagonists useful in combination with a selective HDAC8 inhibitor include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide, SPD-424).

In another embodiment, Dynepo gene activated erythropoietin (Anti-anemic; human erythropoietin) is admistered in combination with selective HDAC8 inhibitor compounds.

“Estrogen receptor modulators” refers to compounds that interfere or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinypethoxy]phenyl]-2H-1-benzopyran-3-yl]-phenyl-2,2-dimethylpr-opanoate, 4,4′-dihydroxybenzophenone-2,4-dinitrophenyl-hydrazone, and SH646. In some embodiments, estrogen receptor modulators are tamoxifen and raloxifene.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl)retinamide, and N-4-carboxyphenyl retinamide.

Other agents that are used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which are used in combination with a selective HDAC8 inhibitor include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, lnanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

“Cytotoxic/cytostatic agents” refer to compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of histone deacetylase, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine-(chloro)platinum(II)]-tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunorubicin (see WO 00/50032).

Examples of microtubulin inhibitors include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)-benzene sulfonamide, anhydrovinblastine, N,N-dimethyl-L-valyl-L-valyl-N-methyl-L-valyl-L-prolyl-L-proline-t-butylamide, TDX258, and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)-ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,′:6,7)colchic(2,3-d)-1,3-dioxol-6-one, 2,3-(methylenedioxy)-5-methyl-7-hydroxy-8-methoxybenzo[c]-phenanthridinium, 6,9-bis[(2-aminoethyl)-amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2-, 1-c]quinolin-7-one, and dimesna.

“Antiproliferative agents” include antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxy-cytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]-glycylamino]-L-glycero-B-L-manno-heptopyranosyl]-adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetra cyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, and 3-aminopyridine-2-carboxaldehyde thiosemicarbazone. “Antiproliferative agents” also includes monoclonal antibodies to growth factors, other than those listed under “angiogenesis inhibitors”, such as trastuzumab, and tumor suppressor genes, such as p53, which are delivered via recombinant virus-mediated gene transfer (see U.S. Pat. No. 6,069,134, for example).

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). Examples of prenyl-protein transferase inhibiting compounds include (±)-6-[amino(4-chlorophenyl)(1-methyl-1H-imidazol-5-yl)methyl]-4-(3-chlorophenyl)-1-methyl-2(1H)-quinolinone, (−)-6-[amino(4-chloropheny-1)(1-methyl-1H-imidazol-5-yl)methyl]-4-(3-chlorophenyl)-1-methyl-2(1H)-quino linone, (+)-6-[amino (4-chlorophenyl)(1-methyl-1H-imidazol-5-yl)methyl]-4-(3-chlorophenyl)-1-methyl-2(1H)-quinolinone, 5(S)-n-butyl-1-(2,3-dimethyl-phenyl)-4-[1-(4-cyanobenzyl)-5-imidazolylmethyl]-2-piperazinone, (S)-1-(3-chlorophenyl)-4-[1-(4-cyanobenzyl)-5-imidazolylmethyl]-5-[2-(ethanesulfonyl)-methyl)-2-piperazinone, 5(S)-n-butyl-1-(2-methylphenyl)-4-[1-(4-cyanobenzyl)-5-imidazolylmethyl]-2-piperazinone, 1-(3-chlorophenyl)-4-[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-yl-ethyl)carbamoyl]-piperidine, 4-{5-[4-hydroxymethyl-4-(4-chloropyridin-2-ylmethyl)-piperidine-1-ylmethyl]-2-methylimidazol-1-ylmethyl}benzonitrile, 4-{5-[4-hydroxymethyl-4-(3-chlorobenzyl)-piperidine-1-ylmethyl]-2-methylimidazol-1-ylmethyl}benzonitrile, 4-{3-[4-(2-oxo-2H-pyridin-1-yl)benzyl]-3H-imidazol-4-ylmethyl}benzonitrile, 4-{3-[4-(5-chloro-2-oxo-2H-[1,2]bipyridin-5′-ylmethyl]-3H-imidazol-4-ylmethyl}benzonitrile, 4-{3-[4-(2-oxo-2H-[1,2]bipyridin-5′-ylmethyl]-3H-imidazol-4-ylmethyl}benzonitrile, 4-{3-(2-oxo-1-phenyl-1,2-dihydropyridin-4-ylmethyl)-3H-imidazol-4-ylmethyl}benzonitrile, 18,19-dihydro-19-oxo-5H,17H-6, 10:12,16-dimetheno-1H-imidazo[4,3-c][1,11,4]dioxa-azacyclononadecine-9-carbonitrile, (±)-19,20-dihydro-19-oxo-5H-18,21-ethano-12,14-etheno-6,10-metheno-22H-benzo[d]imidazo[4,3-k][1,6,9,12]-oxatriaza-cyclooctadecine-9-carbonitrile, 19,20-dihydro-19-oxo-5H,17H-18,21-ethano-6,10: 12,16-dimetheno-22H-imidazo[3,4-h][1,8,11,14]oxatriazacyclo-eicosine-9-carbonitrile, and (±)-19,20-dihydro-3-methyl-19-oxo-5H-18,21-ethano-12,14-etheno-6,10-metheno-22H-benzo[d]imidazo[4,3-k][1,6,9,12]oxa-triazacyclooctadecine-9-carbonitrile.

For an example of the role of a prenyl-protein transferase inhibitor on angiogenesis see J. Of Cancer, Vol. 35, No. 9, pp. 1394-1401 (1999).

Examples of HIV protease inhibitors include amprenavir, abacavir, CGP-73547, CGP-61755, DMP-450, indinavir, nelfinavir, tipranavir, ritonavir, saquinavir, ABT-378, AG 1776, and BMS-232, 632. Examples of reverse transcriptase inhibitors include delaviridine, efavirenz, GS-840, HB Y097, lamivudine, nevirapine, AZT, 3TC, ddC, and ddI. It has been reported (Nat. Med.;8(3):225-32, 2002) that HIV protease inhibitors, such as indinavir or saquinavir, have potent anti-angiogenic activities and promote regression of Kaposi sarcoma.

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR20), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib, valecoxib, and rofecoxib, carboxyamidotriazole, combretastatin A-4, squalamine, 6-O-chloroacetyl-carbonyl)-fumagillol, thalidomide, angiostatin, troponin-1, angiotensin II antagonists (see Fernandez et at., J. Lab. Clin. Med. 105: 141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other examples of angiogenesis inhibitors include, but are not limited to, endostatin, ukrain, ranpirnase, IM862,5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]-methyl]-1H-1,2,3-triazole-4-carboxamide, CM101, squalamine, combretastatin, RP14610, NX31838, sulfated mannopentose phosphate, 7,7-(carbonyl-bis[imino-N-methyl-4,2-pyrrolocarbonyl-imino[N-methyl-4,2-pyrrole]-carbonylimino]-bis-(1,3-naphthalene disulfonate), and 3-[(2,4-dimethylpyrrol-5-yl)methylene]-2-indolinone (SU5416).

“Inhibitors of cell proliferation and survival signaling pathway” refer to pharmaceutical agents that inhibit cell surface receptors and signal transduction cascades downstream of those surface receptors. Such agents include inhibitors of inhibitors of EGFR (for example gefitinib and erlotinib), inhibitors of ERB-2 (for example trastuzumab), inhibitors of IGFR, inhibitors of CD20 (rituximab), inhibitors of cytokine receptors, inhibitors of MET, inhibitors of PDK (for example LY294002), serine/threonine kinases, inhibitors of Rafkinase (for example BAY-43-9006), inhibitors of MEK (for example CI-1040 and PD-098059) and inhibitors of mTOR (for example Wyeth CCI-779 and Ariad AP23573). Such agents include small molecule inhibitor compounds and antibody antagonists.

“Apoptosis inducing agents” include, but not limited to, activators of TNF receptor family members (including the TRAIL receptors).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include, but not limited to, tyrosine kinase inhibitors such as inhibitors of c-Kit, Eph, PDGF, Flt3, Lck, Btk, and c-Met. Further agents include inhibitors of RTKs shown as described by Bume-Jensen and Hunter, 2001, Nature 411: 355-365. Examples of “tyrosine kinase inhibitors” include, but not limited to, N-(trifluoromethylphenyl)-5-methylisoxazol-4-carboxamide, 3-[(2,4-dimethylpyrrol-5-yl)methylidenypindolin-2-one, 17-(allylamino)-17-demethoxygeldanamycin, 4-(3-chloro-4-fluorophenylamino)-7-methoxy-6-[3-(4-morpholinyl)propoxyl]-quinazoline, N-(3-ethynylphenyl)-6,7-bis(2-methoxyethoxy)-4-quinazolinamine, BIBX1382,2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, ST1571, CEP2563, 4-(3-chlorophenylamino)-5,6-dimethyl-7-H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, SU11248, STI571A, N-4-chlorophenyl-4-(4-pyridylmethyl)-1-phthalazinamine, and EMD121974.

HDAC inhibitors are also useful in combination with platelet fibrinogen receptor (GP Iib/IIIa) antagonists, such as tirofiban, to inhibit metastasis of cancerous cells. Tumor cells activate platelets largely via thrombin generation. This activation is associated with the release of VEGF. The release of VEGF enhances metastasis by increasing extravasation at points of adhesion to vascular endothelium (Amirkhosravi, 1999, Platelets 10: 285-292). Therefore, HDAC inhibitors serve to inhibit metastasis, in combination with GP Iib/IIIa) antagonists. Examples of other fibrinogen receptor antagonists include abciximab, eptifibatide, sibrafiban, lamifiban, lotrafiban, cromofiban, and CT50352.

As used above, “integrin blockers” refers to compounds which selectively antagonize, inhibit or counteract binding of a physiological ligand to the αᵥβ₃ integrin, to compounds which selectively antagonize, inhibit or counter-act binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆; αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Commercially available anti-cancer agents which are used in combination with an HDAC8 selective agent disclosed herein include, but are not limited to: abarelix (Plenaxis®); aldesleukin (Prokine®); Aldesleukin (Proleukin®); Alemtuzumab (Campath®); alitretinoin (Panretin®); allopurinol(Zyloprim®); altretamine (Hexylen®); amifostine (Ethyol®); anastrozole (Arimidex®); arsenic trioxide (Trisenox®); asparaginase (Elspar®); azacitidine (Vidaza®); bevacizumab (Avastin®); bexarotene (Targretin®); bleomycin (Blenoxane®); bortezomib (Velcade®); busulfan (Busulfex®); busulfan (Myleran®); calusterone (Methosarb®); capecitabine Xeloda®); carboplatin Paraplatin®); carmustine (BCNU, BiCNU); carmustine (Gliadel®); celecoxib (Celebrex®); cetuximab (Erbitux®); chlorambucil (Leukeran®); cisplatin (Platinol®); cladribine (Leustatin®); clofarabine (Clolar®); cyclophosphamide (Cytoxan®); cytarabine (Cytosar-U®); cytarabine liposomal (DepoCyt); dacarbazine (DTIC-Dome); dactinomycin(actinomycin D, Cosmegen®); Darbepoetin alfa (Aranesp®); dasatinib (Sprycel®); daunorubicin liposomal (DanuoXome); daunorubicin (daunomycin, Daunorubicin®); daunorubicin(daunomycin, Cerubidine®); decitabine (Dacogen®); denileukin (Ontak®); dexrazoxane (Zinecard®); docetaxel (Taxotere®); doxorubicin (Adriamycin®); doxorubicin liposomal (Doxil®); dromostanolone propionate; epirubicin (Ellence®); Epirubicin; Epoetin alfa (EPOGEN®); erlotinib (Tarceva®); estramustine (Emcyt®); etoposide phosphate (Etopophos®); etoposide (VP-16; Vepesid®); exemestane (AROMASIN®); fentanyl citrate (Fentora®); Filgrastim (Neupogen®); floxuridine (FUDR); fludarabine (Fludara®); fluorouracil (5-FU, Adrucil®); fulvestrant (Faslodex®); gefitinib (Iressa®); gemcitabine (Gemzar®); gemtuzumab ozogamicin (Mylotarg®); goserelin acetate (Zoladex®); histrelin acetate (Histrelin®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femora®); leucovorin (Leucovorin®); Leuprolide Acetate (Eligard®); levamisole (Ergamisol®); lomustine, CCNU CeeBU®); meclorethamine(nitrogen mustard, Mustargen®); megestrol acetate (Megace®); melphalan (Alkeran®); mercaptopurine (6-MP, Purinethol®); mesna (Mesnex®); methotrexate (Rheumatrex®, Trexall®); methoxsalen (Uvadex®); mitomycin C (Mutamycin®); mitomycin C (Mitozytrex®); mitotane (Lysodren®); mitoxantrone (Novantrone®); nandrolone phenpropionate (Durabolin-50); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); panitumumab (Vectibix®); pegademase (Adagen®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); rituximab (Rituxan®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide (VM-26, Vumon®); testolactone (Teslac®); thalidomide (Thalomid®); thioguanine (6-TG, Thioguanine®); thiotepa (Thioplex®); topotecan (Hycamtin®); toremifene (Fareston®); Tositumomab (Bexxar®); Tositumomab/I-131 tositumomab (Bexxar®); trastuzumab (Herceptin®); tretinoin (ATRA, Vesanoid®); Uracil Mustard; valrubicin (Valstar®); vinblastine (Velban®); vincristine (Oncovin®); vinorelbine (Navelbine®); vorinostat (Zolinza®); zoledronate (Zometa®); and zoledronic acid (Zometa®).

In some embodiments, the HDAC8 selective compounds described herein are used in combination with gene therapy for the treatment of cancer. For an overview of genetic strategies to treating cancer see Hall et al. (Am J Hum Genet. 61:785-789, 1997) and Kufe et al. (Cancer Medicine, 5th Ed, pp 876-889, B C Decker, Hamilton 2000). Gene therapy is used to deliver any tumor suppressing gene. Examples of such genes include, but are not limited to, p53, which are delivered via recombinant virus-mediated gene transfer, Duc-4, NF-I, NF-2, RB, WT1, BRCA1, BRCA2, a uPA/uPAR antagonist (“Adenoviras-Mediated Delivery of a uPA/uPAR Antagonist Suppresses Angiogenesis-Dependent Tumor Growth and Dissemination in Mice,” Gene Therapy, August 1998, 5(8): 1105-13), and interferon-γ (J. Immunol. 2000; 164:217-222).

In other embodiments, the HDAC8 selective compounds described herein are administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

In some embodiments, the HDAC8 selective compounds described herein are employed in conjunction with anti-emetic agents to treat nausea or emesis, including acute, delayed, late-phase, and anticipatory emesis, which result from the use of a HDAC8 selective compound described herein, alone or with radiation therapy. For the prevention or treatment of emesis, a HDAC8 selective compound described herein is used in conjunction with anti-emetic agents, such as, but not limited to: neurokinin-1 receptor antagonists, 5HT3 receptor antagonists (such as ondansetron, granisetron, tropisetron, Palonosetron, and zatisetron), GABA_{B} receptor agonists (such as baclofen), corticosteroids (such as dexamethasone, prednisone, prednisolone, dopamine antagonists (such as, but not limited to, domperidone, droperidol, haloperidol, chlorpromazine, promethazine, prochlorperazine, metoclopramide), antihistamines (H1 histamine receptor antagonists, such as but not limited to, cyclizine, diphenhydramine, dimenhydrinate, meclizine, promethazine, hydroxyzine), cannabinoids (such as but not limited to, cannabis, marinol, dronabinol), and others (such as, but not limited to, trimethobenzamide; ginger, emetrol, propofol).

In one embodiment, an anti-emesis agent selected from among a neurokinin-1 receptor antagonist, a 5HT3 receptor antagonist and a corticosteroid is administered as an adjuvant for the treatment or prevention of emesis that results upon administration of the instant compounds.

In other embodiments, the HDAC8 selective compounds described herein are administered with an agent useful in the treatment of anemia. Such an anemia treatment agent is, for example, a continuous eythropoiesis receptor activator (such as epoetin-a).

In other embodiments, the HDAC8 selective compounds described herein are administered with an agent useful in the treatment of neutropenia. Examples of agents useful in the treatment of neutropenia include, but are not limited to, a hematopoietic growth factor which regulates the production and function of neutrophils such as a human granulocyte colony stimulating factor, (G-CSF). Examples of a G-CSF include filgrastim.

In some embodiments, the HDAC8 selective compounds described herein are administered with an immunologic-enhancing drug, such as levamisole, bacillus Calmette-Guerin, octreotide, isoprinosine and Zadaxin.

In other embodiments, the HDAC8 selective compounds described herein are useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel®), pamidronate (Aredia®), alendronate (Fosamax®), risedronate(Actonel®), zoledronate (Zometa™, ibandronate (Boniva®), incadronate or cimadronate, clodronate, EB-1053, minodronate, neridronate, piridronate and tiludronate including any and all pharmaceutically acceptable salts, derivatives, hydrates and mixtures thereof.

In other embodiments, the HDAC8 selective compounds described herein are useful for treating breast cancer in combination with aromatase inhibitors. Examples of aromatase inhibitors include but are not limited to: anastrozole, letrozole and exemestane.

In some embodiments, the HDAC8 selective compounds described herein are useful for treating or preventing cancer in combination with siRNA or RNAi therapeutics.

“DNA methyltransferase inhibitor” refers to compounds which inhibit the methylation of the DNA base cytosine at the C-5 position of that base by the DNA methyltransferase enzyme. In some embodiments, DNA methyltransferase inhibitors include 5-azacytosine and Zebularine®.

### Radiation Therapy

Radiotherapy, also called radiation therapy, is the treatment of cancer and other diseases with ionizing radiation. Ionizing radiation deposits energy that injures or destroys cells in an area being treated (a “target tissue”) by damaging their genetic material, making it impossible for these cells to continue to grow. Although radiation damages both cancer cells and normal cells, the latter are better able to repair themselves and function properly. Radiotherapy is used to treat localized solid tumors, such as cancers of the skin, tongue, larynx, brain, breast, prostate, colon, uterus and/or cervix. It is also used to treat leukemia and lymphoma (cancers of the blood-forming cells and lymphatic system, respectively).

A technique for delivering radiation to cancer cells is to place radioactive implants directly in a tumor or body cavity. This is called internal radiotherapy (brachytherapy, interstitial irradiation, and intracavitary irradiation are types of internal radiotherapy.) Using internal radiotherapy, the radiation dose is concentrated in a small area, and the patient stays in the hospital for a few days. Internal radiotherapy is frequently used for cancers of the tongue, uterus, prostate, colon, and cervix.

The term “radiotherapy” or “ionizing radiation” include all forms of radiation, including but not limited to α, β, and γ radiation and ultra violet light. Radiotherapy with or without concurrent or sequential chemotherapy is an effective modality for head and neck, breast, skin, anogenital cancers, and certain nonmalignant diseases such as keloid, desmoid tumor, hemangioma, arteriovenous malformation, and histocytosis X.

Provided are methods of using at least one histone deacetylase inhibitor to reduce side effect caused by at least one other therapeutic treatment, such as radiation-induced normal tissue fibrosis or chemotherapy-induced tissue necrosis, and the methods provided herein also synergistically inhibit tumor cell growth with radiotherapy and other anti-cancer agents.

### Growth Hormone Secretagogues

In some embodiments, a selective inhibitor of HDAC8 is used in combination with one or more growth hormone secretagogues including, but not limited to, arginine, L-3,4-dihydroxyphenylalanine (1-Dopa), glucagon, vasopressin, PACAP (pituitary adenylyl cyclase activating peptide), muscarinic receptor agonists and a synthethic hexapeptide, GHRP (growth hormone releasing peptide).

### Agents for Treating Autoimmune Diseases, Inflammatory Diseases, or Allergy Diseases

In one embodiment, where the subject is suffering from or at risk of suffering from an autoimmune disease, an inflammatory disease, or an allergy disease, a selective HDAC8 inhibitor compound is administered in any combination with one or more of the following therapeutic agents: immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics.

In one embodiment, selective HDAC8 inhibitor compounds described herein, or compositions and medicaments that include the selective HDAC8 inhibitor compounds described herein, are administered to a patient in combination with an anti-inflammatory agent including, but not limited to, non-steroidal anti-inflammatory drugs (NSAIDs) and corticosteroids (glucocorticoids).

NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, COX-2 specific inhibitors (such as, but not limited to, celecoxib, rofecoxib, valdecoxib, parecoxib, etoricoxib, CS-502, JTE-522, L-745,337 and NS398).

Combinations with NSAIDs, which are selective COX-2 inhibitors, are contemplated herein.

Compounds that have been described as selective COX-2 inhibitors and are therefore useful in the methods or pharmaceutical compositions describede herein include, but are not limited to, celecoxib, rofecoxib, lumiracoxib, etoricoxib, valdecoxib, and parecoxib, or a pharmaceutically acceptable salt thereof.

Corticosteroids, include, but are not limited to: betamethasone, prednisone, alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

In one embodiment, HDAC8 selective inhibitors are administered in combination with leukotriene receptor antagonists including, but are not limited to, BAY u9773, Cuthbert et at EP 00791576 (published 27 Aug. 1997), DUO-LT (Tsuji et al, Org. Biomol. Chem., 1, 3139-3141, 2003), zafirlukast (Accolate®), montelukast (Singulair®), prankulast (Onon®), and derivatives or analogs thereof.

### Kits/Articles of Manufacture

For use in the therapeutic applications described herein, kits and articles of manufacture are also described herein. Such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers are formed from a variety of materials such as glass or plastic.

The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease, disorder, or condition that would benefit by inhibition of HDAC activity, or in which HDAC is a mediator or contributor to the symptoms or cause.

For example, the container(s) include one or more compounds described herein, optionally in a composition or in combination with another agent as disclosed herein. The container(s) optionally have a sterile access port (for example a container that is an intravenous solution bag or a vial having a stopper pierceable by a hypodermic injection needle). Such kits optionally comprising a compound with an identifying description or label or instructions relating to its use in the methods described herein.

A kit will include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also be included.

A label is attached on or associated with the container. A label is attached on a container when letters, numbers or other characters forming the label are attached, molded or etched into the container itself; a label is associated with a container when it is present within a receptacle or carrier that also holds the container, e.g., as a package insert. A label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. The pack or dispenser device is accompanied by instructions for administration. The pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. Compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein. The starting materials and reagents used for the synthesis of the compounds described herein are synthesized or obtained from commercial sources, such as, Sigma-Aldrich, Fluka, Acros Organics, Alfa Aesar, Bachem and the like.

### Example 1

### Synthesis of (E)-3-(2-(4-chlorophenoxy)phenyl)-N-hydroxyacrylamide (6)

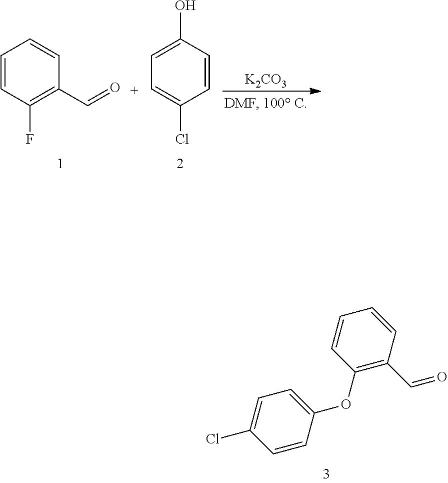

Step 1: A mixture of 2-fluoro-benzaldehyde (1, 7.0 g, 56.4 mmol), 4-chlorophenol (2, 7.25 g, 56.4 mmol) and K₂CO₃ (12.0 g, 85 mmol) in 50 mL DMF was heated overnight at 100° C. Progress of the reaction was monitored by LC/MS. After reaction completion, the reaction mixture was cooled, poured into water (30 mL) and then extracted twice with EtOAc. The EtOAc layers were combined and washed with water, then brine and dried with MgSO₄. After filtration and concentration, the crude material was purified by flash chromatography (hexane/EtOAc: 0-100%) to provide 9.0 g (69% yield) of 2-(4-chlorophenoxy)benzaldehyde (3).

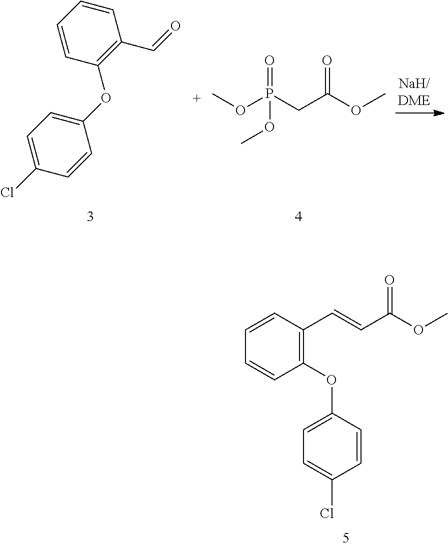

Step 2: To a solution of 2-(4-chlorophenoxy)benzaldehyde (3, 0.5 g, 2.15 mmol) and trimethyl phosphonoacetate (4, 0.47 g, 2.6 mmol) in 20 mL of DMF was added NaH (95%) (62 mg, 2.6 mmol). The mixture was stirred overnight at 100° C. The DME was evaporated, then water was added to quench the reaction and extracted twice with EtOAc. The EtOAc layers were combined, washed with water, then brine and dried with MgSO₄. After filtration and concentration, 0.57 g (90% yield: >90% purity by UV254) of (E)-methyl 3-(2-(4-chlorophenoxy)phenyl)acrylate (5) was isolated and used without further purification.

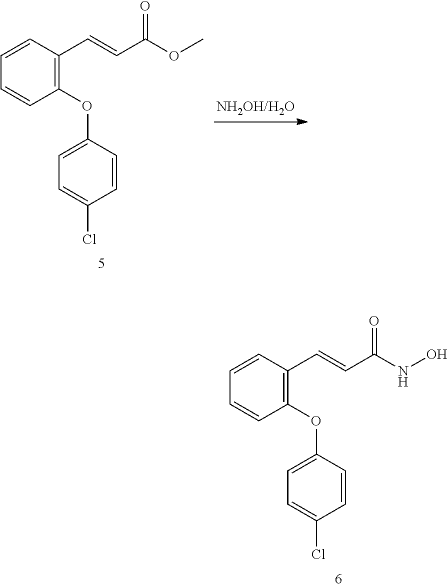 [SMILES: COC(=O)/C=C/c1ccccc1Oc1ccc(Cl)cc1 ; O=C(/C=C/c1ccccc1Oc1ccc(Cl)cc1)NO]

Step 3: To a cooled solution of (E)-methyl 3-(2-(4-chlorophenoxy)phenyl)acrylate (5, 0.29 g, 1.0 mmol) in IPA (5 mL) was added 50% NH₂OH/H₂O (1.0 g, 30 mmol), and then 1N NaOH (2 mmol, 2 mL). The reaction mixture was stirred for 1 hr at 0° C., then acidified to pH=7, diluted with water, and then extrated with EtOAc (3×50 mL). The combined organic layers were washed with brine and dried with MgSO₄. After filtration and evaporation, 0.24 g (84% yield) of (E)-3-(2-(4-chlorophenoxy)phenyl)-N-hydroxyacrylamide (6) was isolated. The crude material was purified by HPLC. EM (calc): 289.05; MS (M+1H)=290.0.

### Example 2

### Synthesis of (E)-N-Hydroxy-3-(2-phenoxyphenyl)acrylamide (7)

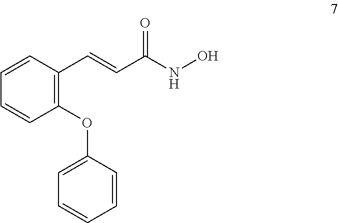 [SMILES: O=C(/C=C/c1ccccc1Oc1ccccc1)NO]

The title compound was synthesized as described in Example 1. EM (calc): 255.09; MS (M+1H)=256.5.

### Example 3

### Synthesis of (E)-3-(2-(p-Tolyloxy)phenyl)-N-hydroxyacrylamide (8)

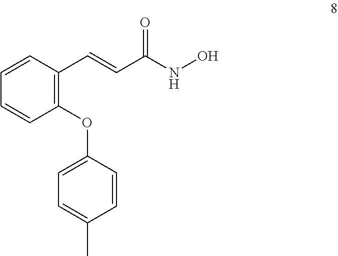 [SMILES: Cc1ccc(Oc2ccccc2/C=C/C(=O)NO)cc1]

The title compound was synthesized as described in Example 1. EM (calc): 269.11; MS (M+1H)=270.5.

### Example 4

### Synthesis of (E)-3-(2-(4-Fluorophenoxy)phenyl)-N-hydroxyacrylamide (9)

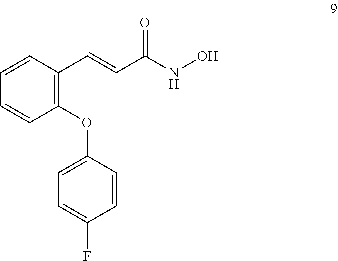 [SMILES: O=C(/C=C/c1ccccc1Oc1ccc(F)cc1)NO]

The title compound was synthesized as described in Example 1. EM (calc): 273.08; MS (M+1H)=274.0.

### Example 5

### Synthesis of (E)-N-hydroxy-3-(2-(4-methoxyphenoxy)phenyl)acrylamide (10)

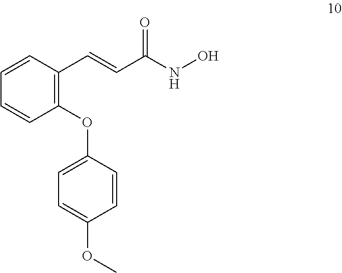 [SMILES: COc1ccc(Oc2ccccc2/C=C/C(=O)NO)cc1]

The title compound was synthesized as described in Example 1. EM (calc): 285.1; MS (M+1H)=286.0.

### Example 6

### Synthesis of (E)-3-(2-(3-Chlorophenoxy)phenyl)-N-hydroxyacrylamide (11)

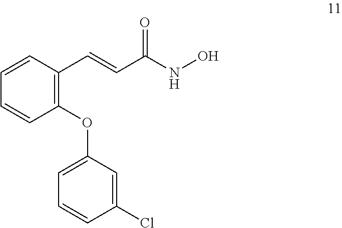 [SMILES: O=C(/C=C/c1ccccc1Oc1cccc(Cl)c1)NO]

The title compound was synthesized as described in Example 1. EM (calc): 289.05; MS (M+1H)=290.0.

### Example 7

### Synthesis of (E)-3-(2-(3,4-Dichlorophenoxy)phenyl)-N-hydroxyacrylamide (12)

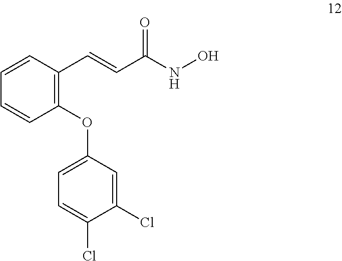 [SMILES: O=C(/C=C/c1ccccc1Oc1ccc(Cl)c(Cl)c1)NO]

The title compound was synthesized as described in Example 1. EM (calc): 323.01; MS (M+1H)=324.5.

### Example 8

### Synthesis of (E)-N-hydroxy-3-(2-(m-tolyloxy)phenyl)acrylamide (13)

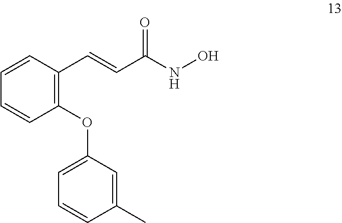 [SMILES: Cc1cccc(Oc2ccccc2/C=C/C(=O)NO)c1]

The title compound was synthesized as described in Example 1. EM (calc): 269.11; MS (M+1H)=270.5.

### Example 9

### Synthesis of (E)-3-(2-(3-Fluorophenoxy)phenyl)-N-hydroxyacrylamide (14)

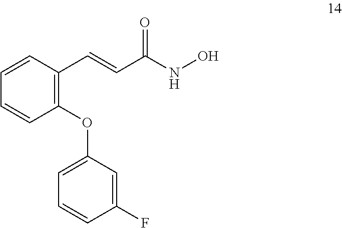 [SMILES: O=C(/C=C/c1ccccc1Oc1cccc(F)c1)NO]

The title compound was synthesized as described in Example 1. EM (calc): 273.08; MS (M+1H)=274.0.

### Example 10

### Synthesis of (E)-N-hydroxy-3-(2-(3-methoxyphenoxy)phenyl)acrylamide (15)

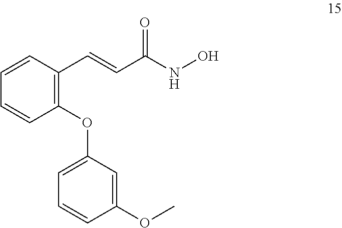 [SMILES: COc1cccc(Oc2ccccc2/C=C/C(=O)NO)c1]

The title compound was synthesized as described in Example 1. EM (calc): 285.1; MS (M+1H)=286.5.

### Example 11

### (E)-N-Hydroxy-3-(2-(pyridin-3-yloxy)phenyl)acrylamide (16)

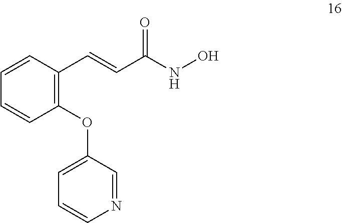 [SMILES: O=C(/C=C/c1ccccc1Oc1cccnc1)NO]

The title compound was synthesized as described in Example 1. EM (calc): 256.08; MS (M+1H)=257.5.

### Example 12

### Synthesis of (E)-N-Hydroxy-3-(2-(pyridin-4-yloxy)phenyl)acrylamide (17)

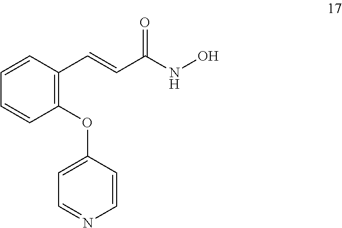 [SMILES: O=C(/C=C/c1ccccc1Oc1ccncc1)NO]

The title compound was synthesized as described in Example 1. EM (calc): 256.08; MS (M+1H)=257.5.

### Example 13

### Synthesis of (E)-3-(2-(1-acetylpiperidin-4-yloxy)phenyl)-N-hydroxyacrylamide (18)

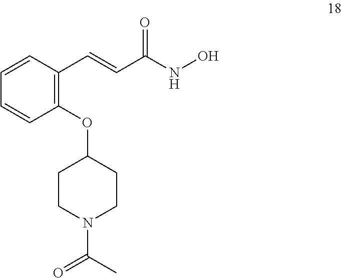 [SMILES: CC(=O)N1CCC(Oc2ccccc2/C=C/C(=O)NO)CC1]

The title compound was synthesized as described in Example 1. EM (calc): 304.14; MS (M+1H)=305.5.

### Example 14

### Synthesis of (E)-N-hydroxy-3-(2-(1-isobutyrylpiperidin-4-yloxy)phenyl)acrylamide (19)

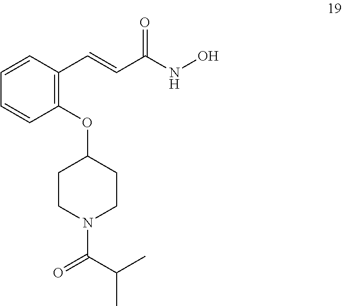 [SMILES: CC(C)C(=O)N1CCC(Oc2ccccc2/C=C/C(=O)NO)CC1]

The title compound was synthesized as described in Example 1. EM (calc): 332.17; MS (M+1H)=333.5.

### Example 15

### Synthesis of (E)-3-(2-(1-benzoylpiperidin-4-yloxy)phenyl)-N-hydroxyacrylamide (20)

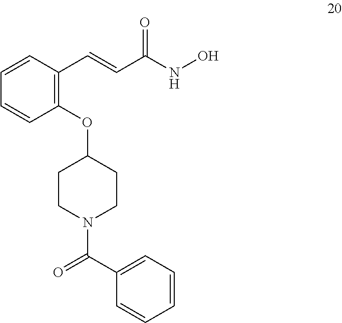 [SMILES: O=C(/C=C/c1ccccc1OC1CCN(C(=O)c2ccccc2)CC1)NO]

The title compound was synthesized as described in Example 1. EM (calc): 366.16; MS (M+1H)=367.0.

### Example 16

### Synthesis of (E)-N-hydroxy-3-(2-(1-nicotinoylpiperidin-4-yloxy)phenyl)acrylamide (21)

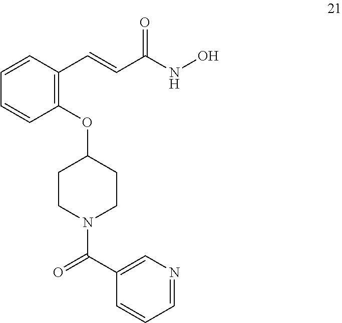 [SMILES: O=C(/C=C/c1ccccc1OC1CCN(C(=O)c2cccnc2)CC1)NO]

The title compound was synthesized as described in Example 1. EM (calc): 367.15; MS (M+1H)=368.0.

### Example 17

### Synthesis of (E)-3-(2-(1-(furan-2-carbonyl)piperidin-4-yloxy)phenyl)-N-hydroxyacrylamide (22)

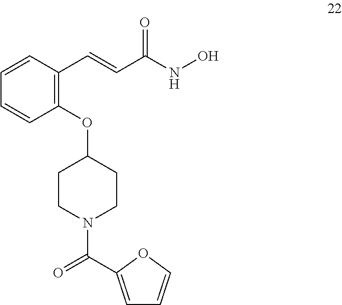 [SMILES: O=C(/C=C/c1ccccc1OC1CCN(C(=O)c2ccco2)CC1)NO]

The title compound was synthesized as described in Example 1. EM (calc): 356.14; MS (M+1H)=357.0.

### Example 18

### Synthesis of (S,E)-3-(2-(1-acetylpiperidin-3-yloxy)phenyl)-N-hydroxyacrylamide (23)

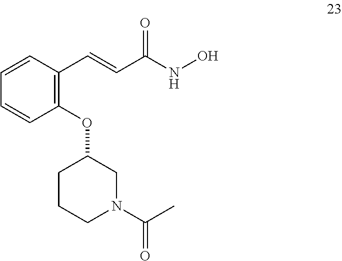

The title compound was synthesized as described in Example 1. EM (calc): 304.14; MS (M+1H)=305.5.

### Example 19

### Synthesis of (S,E)-N-hydroxy-3-(2-(1-isobutyrylpiperidin-3-yloxy)phenyl)acrylamide (24)

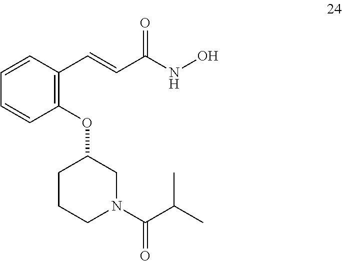

The title compound was synthesized as described in Example 1. EM (calc): 332.17; MS (M+1H)=333.5.

### Example 20

### Synthesis of (S,E)-3-(2-(1-benzoylpiperidin-3-yloxy)phenyl)-N-hydroxyacrylamide (R,E)-3-(2-(1-benzoylpiperidin-3-yloxy)phenyl)-N-hydroxyacrylamide (25)

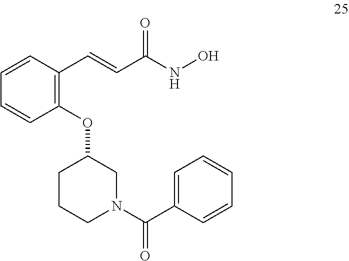

The title compound was synthesized as described in Example 1. EM (calc): 366.16; MS (M+1H)=367.0.

### Example 21

### Synthesis of (S,E)-N-hydroxy-3-(2-(1-nicotinoylpiperidin-3-yloxy)phenyl)acrylamide (26)

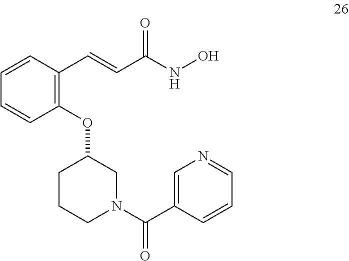

The title compound was synthesized as described in Example 1. EM (calc): 367.15; MS (M+1H)=368.5.

### Example 22

### Synthesis of (S,E)-3-(2-(1-(furan-2-carbonyl)piperidin-3-yloxy)phenyl)-N-hydroxyacrylamide (27)

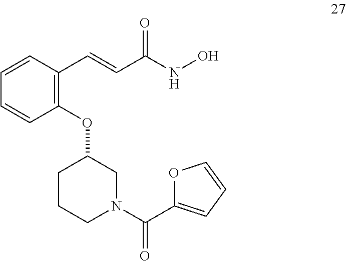

The title compound was synthesized as described in Example 1. EM (calc): 356.14; MS (M+1H)=357.5.

### Example 23

### Synthesis of (R,E)-3-(2-(1-acetylpiperidin-3-yloxy)phenyl)-N-hydroxyacrylamide (28)

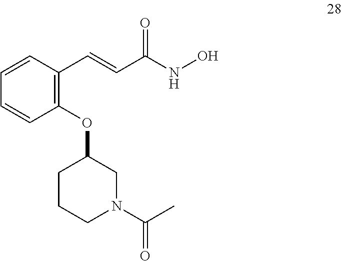

The title compound was synthesized as described in Example 1. EM (calc): 304.14; MS (M+1H)=305.5.

### Example 24

### Synthesis of (R,E)-N-hydroxy-3-(2-(1-isobutyrylpiperidin-3-yloxy)phenyl)acrylamide (29)

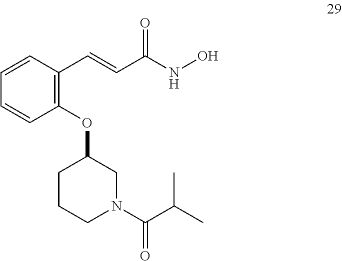

The title compound was synthesized as described in Example 1. EM (calc): 332.17; MS (M+1H)=333.5.

### Example 25

### Synthesis of (R,E)-3-(2-(1-benzoylpiperidin-3-yloxy)phenyl)-N-hydroxyacrylamide (30)

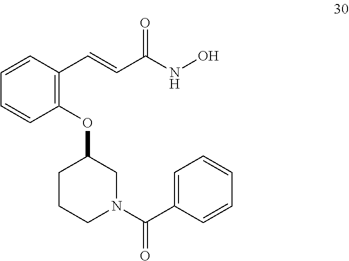

The title compound was synthesized as described in Example 1. EM (calc): 366.16; MS (M+1H)=367.0.

### Example 26

### Synthesis of (R,E)-N-hydroxy-3-(2-(1-nicotinoylpiperidin-3-yloxy)phenyl)acrylamide (31)

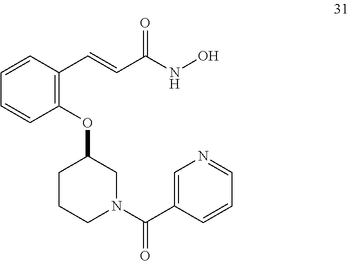

The title compound was synthesized as described in Example 1. EM (calc): 367.15; MS (M+1H)=368.0.

### Example 27

### Synthesis of (R,E)-3-(2-(1-(furan-2-carbonyl)piperidin-3-yloxy)phenyl)-N-hydroxyacrylamide (32)

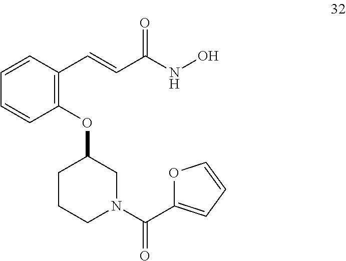

The title compound was synthesized as described in Example 1. EM (calc): 356.14; MS (M+1H)=357.5.

### Example 28

### Synthesis of (E)-N-(4-(4-fluorophenoxy)-3-(3-(hydroxyamino)-3-oxoprop-1-enyl)phenyl)nicotinamide (33)

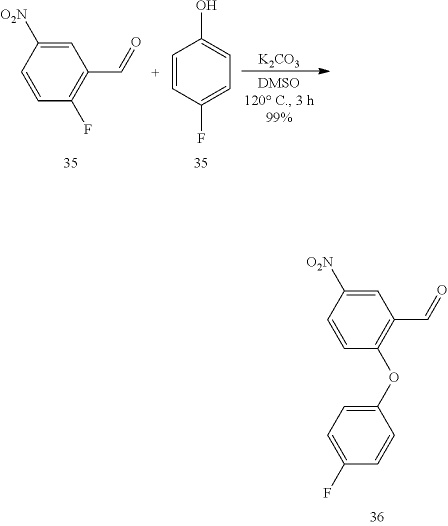

Step 1: To a solution of 2-fluoro-5-nitro-benzaldehyde (34) (676 mg, 4.0 mmol) and 4-fluoroorophenol (35) (537 mg, 4.8 mmol) in DMSO (5 mL) was added K₂CO₃ (1.10 g, 8.0 mmol) at room temperature (“rt”). The resulting mixture was flushed with N₂ and heated in a sealed vessel with stirring at 120° C. for 3 h. After the reaction mixture was cooled to rt and poured into brine, the mixture was extracted with ethyl acetate (35 mL×3). The combined extracts were washed with brine (10 mL×2), dried over anhydrous MgSO₄, filtered, and evaporated to dryness. The residue was purified by a SiO₂ plug (eluted by 15% EtOAc in hexanes) to afford pure 2-(4-fluoro-phenoxy)-5-nitro-benzaldehyde (36) (1.05 g, 4.0 mmol, 99%) as a viscous oil. ESI MS m/z 262.2 (M+H)⁺.

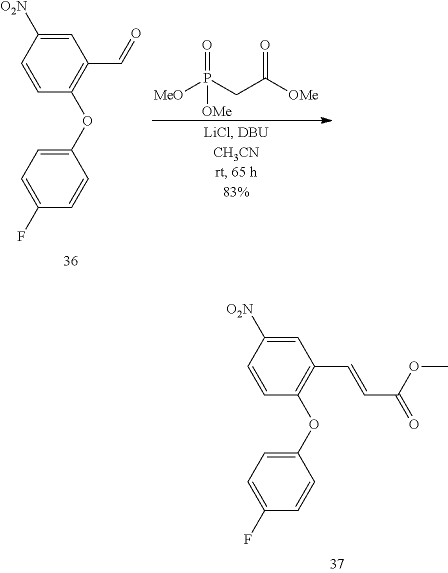 [SMILES: O=Cc1cc([N+](=O)[O-])ccc1Oc1ccc(F)cc1 ; COC(=O)CP(=O)(OC)OC ; COC(=O)/C=C/c1cc([N+](=O)[O-])ccc1Oc1ccc(F)cc1]

Step 2: To a stirred solution of 2-(4-fluoro-phenoxy)-5-nitro-benzaldehyde (36) (469 mg, 1.79 mmol) and trimethyl phosphonoacetate (650 mg, 3.58 mmol) in acetonitrile (20 mL) was added LiCl (228 mg, 5.36 mmol), followed by DBU (0.802 mL, 5.36 mmol) at rt. The resulting mixture was stirred at rt for 65 h. After the reaction mixture was concentrated under reduced pressure, the residue was treated with ethyl acetate (100 mL). The EtOAc solution was washed with 1 M HCl aq. (10 mL×2), sat. NaHCO₃ aq. (10 mL), and brine (10 mL), dried over anhydrous MgSO₄, filtered, and evaporated to dryness. The residue was purified by a SiO₂ plug (eluted by 10% to 25% EtOAc in hexanes) to afford pure 3-[2-(4-fluoro-phenoxy)-5-nitro-phenyl]-acrylic acid methyl ester (37) (478 mg, 1.50 mmol, 83%) as a viscous oil. ESI MS m/z 318.3 (M+H)⁺.

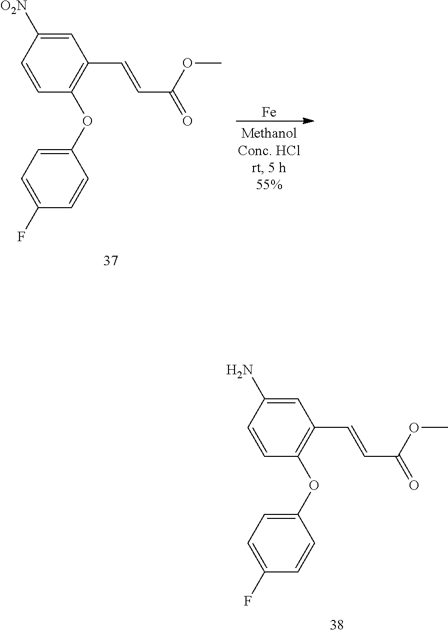

Step 3: To a stirred solution of 3-[2-(4-fluoro-phenoxy)-5-nitro-phenyl]-acrylic acid methyl ester (37) (478 mg, 1.50 mmol) in methanol (10 mL) was added 412 mg (7.5 mmol) of iron powder. To this mixture 10 mL of conc. HCl was added at room temperature. The resulting mixture was stirred at rt for 5 h and then allowed to stand for 1 h. The reaction mixture was filtered through a Buckner funnel to remove iron powder and the resulting solution was evaporated to dryness. It is then treated with ethyl acetate and washed with aqueous sodium bicarbonate and dilute HCl. The organic layer was separated and the aqueous layer was further extracted with EtOAc (30 mL×3). The combined organic phases were washed with brine (10 mL×2), dried over anhydrous MgSO₄, filtered, and evaporated to dryness. The 3-[2-(4-fluoro-phenoxy)-5-amino-phenyl]-acrylic acid methyl ester (38) was isolated as a yellow oil and used without further purification. ESI MS m/z 288.3 (M+H)⁺.

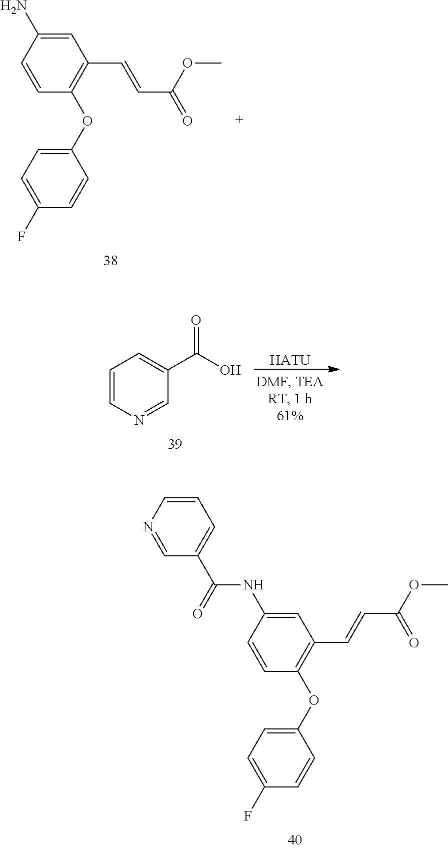

Step 4: To a stirred solution of 3-[2-(4-fluoro-phenoxy)-5-amino-phenyl]-acrylic acid methyl ester (38) (58 mg, 0.20 mmol) and 3-pyridyl carboxylic acid (39) (35 mg, 0.32 mmol) in DMF (2 mL) was added HATU (114 mg, 0.30 mmol), followed by a solution of TEA (0.033 mL, 0.32 mmol) at rt under N₂. The resulting mixture was stirred for 4 h. After the reaction mixture was concentrated under reduced pressure, the residue was treated with ethyl acetate (75 mL). The solution was washed with acid (HCl) and base (NaHCO₃) and brine (10 mL×2), dried over anhydrous MgSO₄, filtered, and evaporated to dryness. The residue was purified by a SiO₂ plug (eluted by 33% to 50% EtOAc in hexanes) to afford 3-{2-(4-fluoro-phenoxy)-5-[(pyridine-3-carbonyl)-amino]-phenyl}-acrylic acid methyl ester (40) (47 mg, 0.12 mmol, 61%) as a viscous oil. ESI MS m/z 393.4 (M+H)⁺.

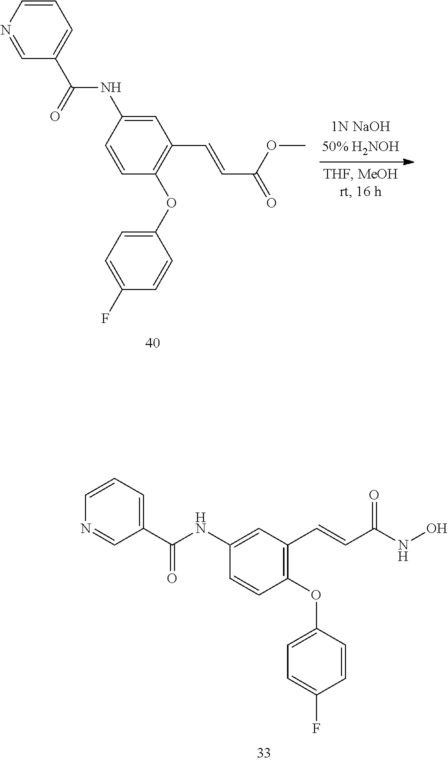 [SMILES: COC(=O)/C=C/c1cc(NC(=O)c2cccnc2)ccc1Oc1ccc(F)cc1 ; O=C(/C=C/c1cc(NC(=O)c2cccnc2)ccc1Oc1ccc(F)cc1)NO]

Step 5: To a stirred solution of 3-{2-(4-fluoro-phenoxy)-5-[(pyridine-3-carbonyl)-amino]-phenyl}-acrylic acid methyl ester (40) (86 mg, 0.22 mmol) in THF (1.2 mL) and MeOH (1.2 mL) was added 50% solution of NH₂OH in water (0.73 mL, 11 mmol) and 1 N NaOH aq. (0.5 mL, 0.5 mmol) at rt. The resulting mixture was stirred at rt for 16 h. After the reaction mixture was concentrated under reduced pressure, the resulting aqueous suspension was diluted with DMSO (1.5 mL) and purified by HPLC to afford N-[4-(4-fluoro-phenoxy)-3-(2-hydroxycarbamoyl-vinyl)-phenyl]-nicotinamide (33) (54 mg, 0.14 mmol, 62%) as a white solid. EM (calc): 393.37; ESI MS m/z 394.1 (M+H)⁺.

### Example 29

### Synthesis of (E)-3-(5-acetamido-2-(4-fluorophenoxy)phenyl)-N-hydroxyacrylamide (41)

The title compound was synthesized as described in Example 28. EM (calc): 330.1; MS (M+1H)=331.0.

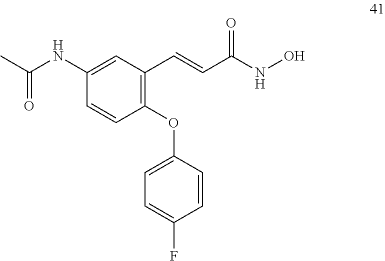 [SMILES: CC(=O)Nc1ccc(Oc2ccc(F)cc2)c(/C=C/C(=O)NO)c1]

### Example 30

### Synthesis of (E)-3-(5-acetamido-2-(3-chlorophenoxy)phenyl)-N-hydroxyacrylamide (42)

The title compound was synthesized as described in Example 28. EM (calc): 346.07; MS (M+1H)=347.0.

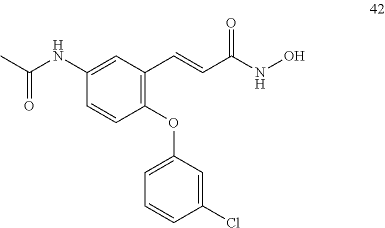 [SMILES: CC(=O)Nc1ccc(Oc2cccc(Cl)c2)c(/C=C/C(=O)NO)c1]

### Example 31

### Synthesis of (E)-N-(4-(3-chlorophenoxy)-3-(3-(hydroxyamino)-3-oxoprop-1-enyl)phenyl)nicotinamide (43)

The title compound was synthesized as described in Example 28. EM (calc): 409.08; MS (M+1H)=410.5.

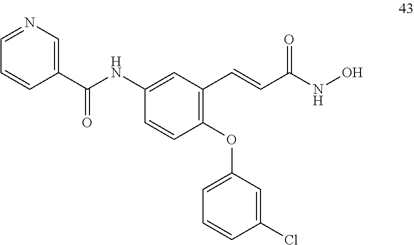 [SMILES: O=C(/C=C/c1cc(NC(=O)c2cccnc2)ccc1Oc1cccc(Cl)c1)NO]

### Example 32

### Synthesis of (E)-N-(4-(3-fluorophenoxy)-3-(3-(hydroxyamino)-3-oxoprop-1-enyl)phenyl)nicotinamide (44)

The title compound was synthesized as described in Example 28. EM (calc): 393.11; MS (M+1H)=394.0.

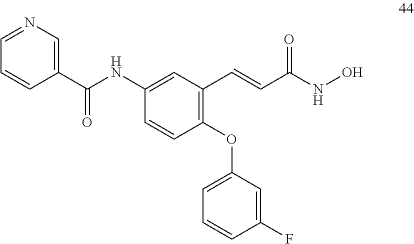 [SMILES: O=C(/C=C/c1cc(NC(=O)c2cccnc2)ccc1Oc1cccc(F)c1)NO]

### Example 33

### Synthesis of (E)-N-(4-(3,4-dichlorophenoxy)-3-(3-(hydroxyamino)-3-oxoprop-1-enyl)phenyl)nicotinamide (45)

The title compound was synthesized as described in Example 28. EM (calc): 443.04; MS (M+1H)=444.5.

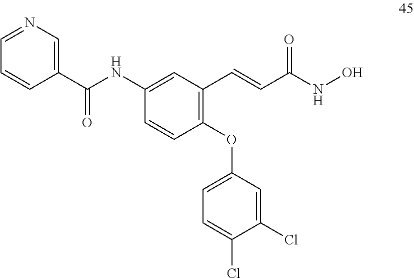 [SMILES: O=C(/C=C/c1cc(NC(=O)c2cccnc2)ccc1Oc1ccc(Cl)c(Cl)c1)NO]

### Example 34

### Synthesis of (E)-3-(5-acetamido-2-(3-fluorophenoxy)phenyl)-N-hydroxyacrylamide (46)

The title compound was synthesized as described in Example 28. EM (calc): 330.10; MS (M+1H)=331.0.

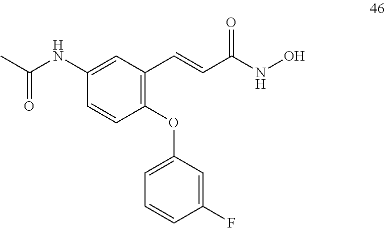 [SMILES: CC(=O)Nc1ccc(Oc2cccc(F)c2)c(/C=C/C(=O)NO)c1]

### Example 35

### Synthesis of (E)-3-(2-(4-fluorophenoxy)-5-(methylsulfonamido)phenyl)-N-hydroxyacrylamide (47)

The title compound was synthesized as described in Example 28. EM (calc): 366.07; MS (M+1H)=367.0.

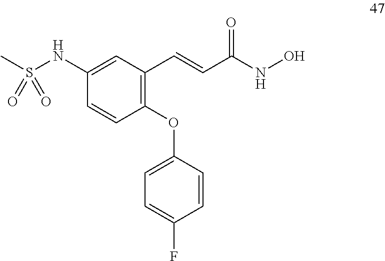 [SMILES: CS(=O)(=O)Nc1ccc(Oc2ccc(F)cc2)c(/C=C/C(=O)NO)c1]

### Example 36

### Synthesis of (E)-3-(2-(3-chlorophenoxy)-5-(methylsulfonamido)phenyl)-N-hydroxyacrylamide (48)

The title compound was synthesized as described in Example 28. EM (calc): 382.04; MS (M+1H)=383.0.

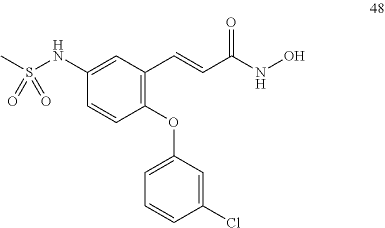 [SMILES: CS(=O)(=O)Nc1ccc(Oc2cccc(Cl)c2)c(/C=C/C(=O)NO)c1]

### Example 37

### Synthesis of (E)-3-(2-(4-fluorophenoxy)-5-(pyridin-3-ylmethylamino)phenyl)-N-hydroxyacrylamide (49)

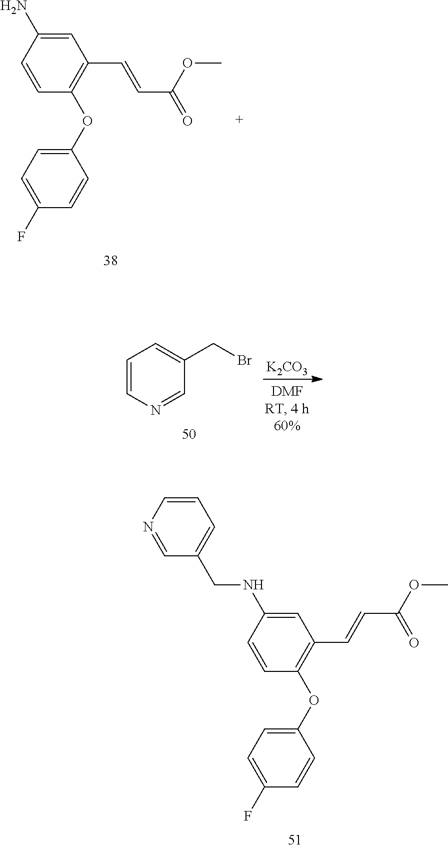

Step 1: To a mixture of 3-[2-(4-fluoro-phenoxy)-5-amino-phenyl]-acrylic acid methyl ester (38) (72 mg, 0.22 mmol) and 3-bromomethyl-pyridine (50) (41 mg, 0.24 mmol) was added 3 mL of DMF and 200 mg K₂CO₃ and stirred at rt for 4 h. After the reaction was completed, the mixture was concentrated under reduced pressure, and the residue was treated with ethyl acetate (75 mL). The ethyl acetate solution was washed with acid (HCl) and base (NaHCO₃) and brine (10 mL×2), dried over anhydrous MgSO₄, filtered, and evaporated to dryness. The residue was purified by a SiO₂ plug (eluted by 33% to 50% EtOAc in hexanes) to afford (E)-methyl 3-(2-(4-fluorophenoxy)-5-(pyridin-3-ylmethylamino)phenyl)acrylate (51) (46 mg, 0.12 mmol, 61%) as a viscous oil. ESI MS m/z 379.4 (M+H)⁺.

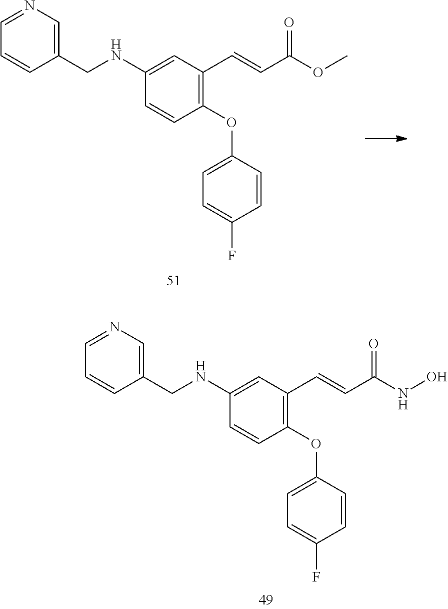 [SMILES: COC(=O)/C=C/c1cc(NCc2cccnc2)ccc1Oc1ccc(F)cc1 ; O=C(/C=C/c1cc(NCc2cccnc2)ccc1Oc1ccc(F)cc1)NO]

Step 2: This compound was prepared from 3-{2-(4-fluoro-phenoxy)-5-[(pyridine-3-ylmethyl)-amino]-phenyl}-acrylic acid methyl ester (51) (84 mg, 0.22 mmol) by following the same procedure described in Example 28, step 5, to provide 43 mg (0.10 mmol, 47%) of (E)-3-(2-(4-fluorophenoxy)-5-(pyridin-3-ylmethylamino)phenyl)-N-hydroxyacrylamide (49) as a white solid after HPLC purification. EM (calc): 379.13; ESI MS m/z 380.1 (M+H)⁺.

### Example 38

### Synthesis of (E)-3-(2-(3-chlorophenoxy)-5-(pyridin-3-ylmethylamino)phenyl)-N-hydroxyacrylamide (52)

The title compound was synthesized as described in Example 37. EM (calc): 395.1; MS (M+1H)=396.1.

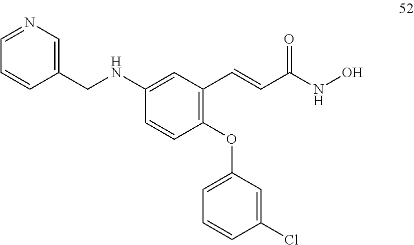 [SMILES: O=C(/C=C/c1cc(NCc2cccnc2)ccc1Oc1cccc(Cl)c1)NO]

### Example 39

### Synthesis of (E)-3-(2-(3-chlorophenoxy)-5-(pyridin-2-ylmethylamino)phenyl)-N-hydroxyacrylamide (53)

The title compound was synthesized as described in Example 37. EM (calc): 395.1; MS (M+1H)=396.0.

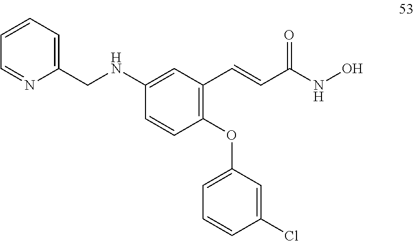 [SMILES: O=C(/C=C/c1cc(NCc2ccccn2)ccc1Oc1cccc(Cl)c1)NO]

### Example 40

### Synthesis of (E)-3-(2-(4-fluorophenoxy)-5-(pyridin-2-ylmethylamino)phenyl)-N-hydroxyacrylamide (54)

The title compound was synthesized as described in Example 37. EM (calc): 379.13; MS (M+1H)=380.5.

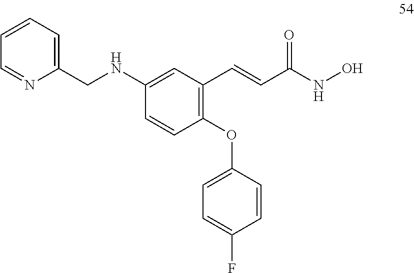 [SMILES: O=C(/C=C/c1cc(NCc2ccccn2)ccc1Oc1ccc(F)cc1)NO]

### Example 41

### Synthesis of (E)-3-(2-(3-fluorophenoxy)-5-(pyridin-3-ylmethylamino)phenyl)-N-hydroxyacrylamide (55)

The title compound was synthesized as described in Example 37. EM (calc): 379.13; MS (M+1H)=380.0.

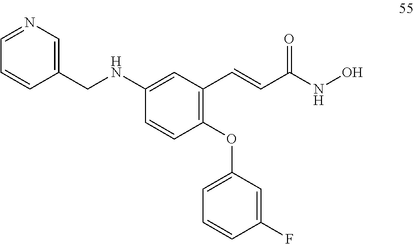 [SMILES: O=C(/C=C/c1cc(NCc2cccnc2)ccc1Oc1cccc(F)c1)NO]

### Example 42

### Synthesis of (E)-3-(2-(3,4-dichlorophenoxy)-5-(pyridin-3-ylmethylamino)phenyl)-N-hydroxyacrylamide (56)

The title compound was synthesized as described in Example 37. EM (calc): 429.06; MS (M+1H)=430.5.

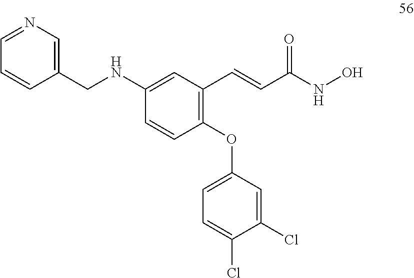 [SMILES: O=C(/C=C/c1cc(NCc2cccnc2)ccc1Oc1ccc(Cl)c(Cl)c1)NO]

### Example 43

### Synthesis of (E)-3-(2-(3-fluorophenoxy)-5-(pyridin-2-ylmethylamino)phenyl)-N-hydroxyacrylamide (57)

The title compound was synthesized as described in Example 37. EM (calc): 379.13; MS (M+1H)=380.5.

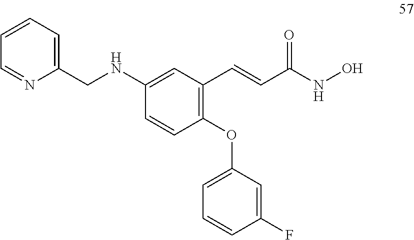 [SMILES: O=C(/C=C/c1cc(NCc2ccccn2)ccc1Oc1cccc(F)c1)NO]

### Example 44

### Synthesis of (E)-3-(2-(3,4-dichlorophenoxy)-5-(pyridin-2-ylmethylamino)phenyl)-N-hydroxyacrylamide (58)

The title compound was synthesized as described in Example 37. EM (calc): 429.06; MS (M+1H)=430.0.

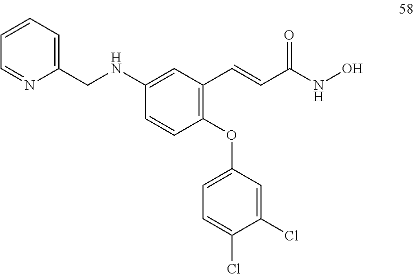 [SMILES: O=C(/C=C/c1cc(NCc2ccccn2)ccc1Oc1ccc(Cl)c(Cl)c1)NO]

### Example 45

### Synthesis of (E)-3-(2-(3-chlorophenoxy)-4-(pyridin-3-ylmethoxy)phenyl)-N-hydroxyacrylamide (59)

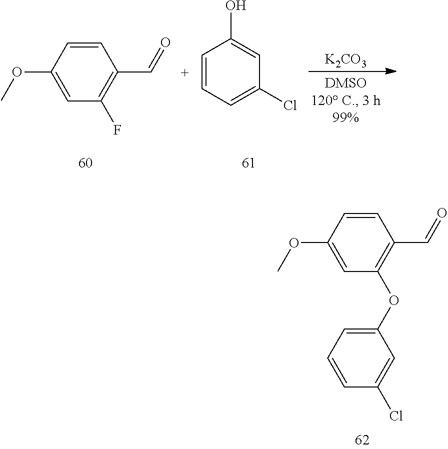

Step 1: To a solution of 2-fluoro-4-methoxy-benzaldehyde (60) (616 mg, 4.0 mmol) and 3-chlorophenol (61) (617 mg, 4.8 mmol) in DMSO (5 mL) was added K₂CO₃ (1.10 g, 8.0 mmol) at rt. The resulting mixture was flushed with N₂ and heated in a sealed vessel with stirring at 120° C. for 3 h. After the reaction mixture was cooled to rt and poured into brine, the mixture was extracted with ethyl acetate (35 mL×3). The combined extracts were washed with brine (10 mL×2), dried over anhydrous MgSO₄, filtered, and evaporated to dryness. The residue was purified by a SiO₂ plug (eluted by 15% EtOAc in hexanes) to afford 2-(3-chlorophenoxy)-4-methoxybenzaldehyde (62) (1.05 g, 4.0 mmol, 99%) as a viscous oil. ESI MS m/z 263.1 (M+H)⁺.

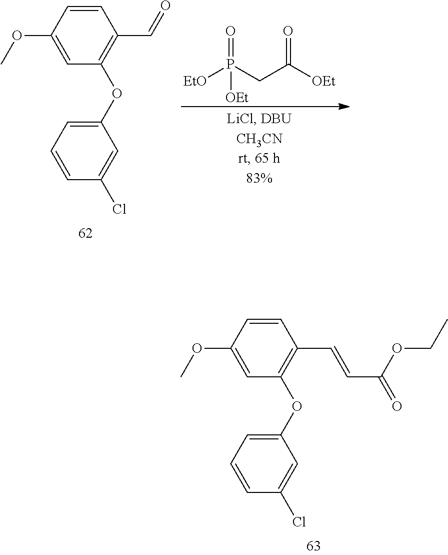 [SMILES: COc1ccc(C=O)c(Oc2cccc(Cl)c2)c1 ; CCOC(=O)CP(=O)(OCC)OCC ; CCOC(=O)/C=C/c1ccc(OC)cc1Oc1cccc(Cl)c1]

Step 2: To a stirred solution of 2-(3-chloro-phenoxy)-4-methoxy-benzaldehyde (62) (469 mg, 1.79 mmol) and triethyl phosphonoacetate (803 mg, 3.58 mmol) in acetonitrile (20 mL) was added LiCl (228 mg, 5.36 mmol), followed by DBU (0.802 mL, 5.36 mmol) at rt. The resulting mixture was stirred at rt for 65 h. After the reaction mixture was concentrated under reduced pressure, the residue was treated with ethyl acetate (100 mL). The EtOAc solution was washed with 1 M HCl aq. (10 mL×2), sat. NaHCO₃ aq. (10 mL), and brine (10 mL), dried over anhydrous MgSO₄, filtered, and evaporated to dryness. The residue was purified by a SiO₂ plug (eluted by 10% to 25% EtOAc in hexanes) to afford (E)-ethyl 3-(2-(3-chlorophenoxy)-4-methoxyphenyl)acrylate (63) (498 mg, 1.50 mmol, 83%) as a viscous oil. ESI MS m/z 333.3 (M+H)⁺.

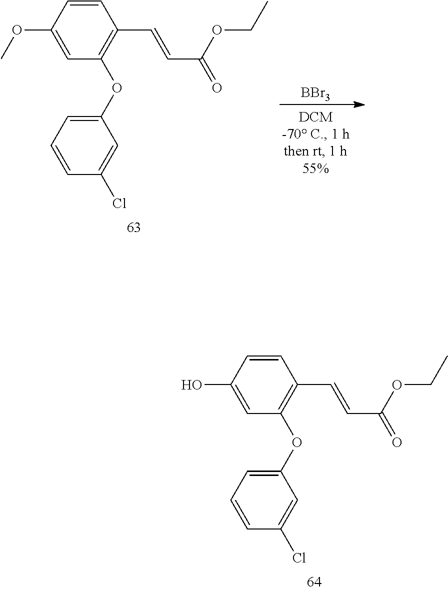

Step 3: To a stirred solution of 3-[2-(3-chloro-phenoxy)-4-methoxy-phenyl]-acrylic acid ethyl ester (63) (498 mg, 1.50 mmol) in DCM (10 mL) was added 1 M solution of BBr₃ in DCM (4.5 mL, 4.5 mmol) at −70° C. under N₂. The resulting mixture was stirred at −70° C. for 1 h and then allowed to warm to rt. After the reaction mixture was stirred at rt for another hour, the reaction was quenched by the addition of sat. aq. NaHCO₃ (10 mL) slowly at 0° C. The mixture was transferred into a separation funnel and the organic layer was separated. The aqueous layer was further extracted with DCM (30 mL×3). The combined organic phases were washed with brine (10 mL×2), dried over anhydrous MgSO₄, filtered, and evaporated to dryness. The residue was purified by a SiO₂ plug (eluted by 10% to 25% EtOAc in hexanes) to afford (E)-ethyl 3-(2-(3-chlorophenoxy)-4-hydroxyphenyl)acrylate (64) (267 mg, 0.84 mmol, 56%) as an off-white powder. ESI MS m/z 318.9 (M+H)⁺.

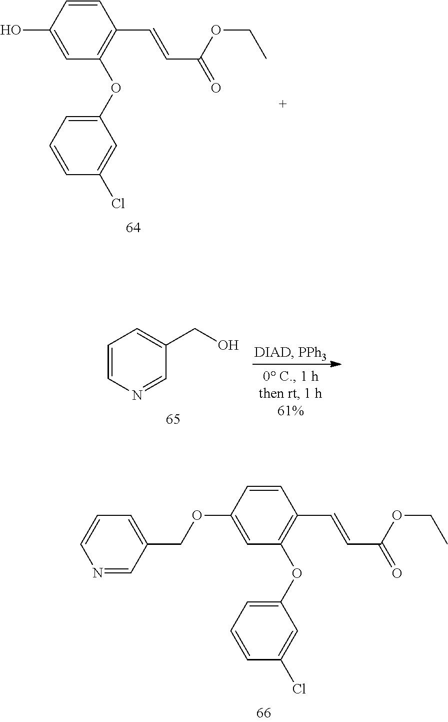

Step 4: To a stirred solution of 3-[2-(3-chloro-phenoxy)-4-hydroxy-phenyl]-acrylic acid ethyl ester (64) (64 mg, 0.20 mmol) and 3-pyridylcarbinol (65) (35 mg, 0.32 mmol) in THF (2 mL) was added Ph₃P (79 mg, 0.30 mmol), followed by a solution of DIAD (0.063 mL, 0.32 mmol) in THF (1 mL) at 0° C. under N₂. The resulting mixture was stirred at 0° C. for 1 h and then at rt for 16 h. After the reaction mixture was concentrated under reduced pressure, the residue was treated with ethyl acetate (75 mL). The solution was washed with brine (10 mL×2), dried over anhydrous MgSO₄, filtered, and evaporated to dryness. The residue was purified by a SiO₂ plug (eluted by 33% to 50% EtOAc in hexanes) to afford (E)-ethyl 3-(2-(3-chlorophenoxy)-4-(pyridin-3-ylmethoxy)phenyl)acrylate (66) (50 mg, 0.12 mmol, 61%) as a viscous oil. ESI MS m/z 410.2 (M+H)⁺.

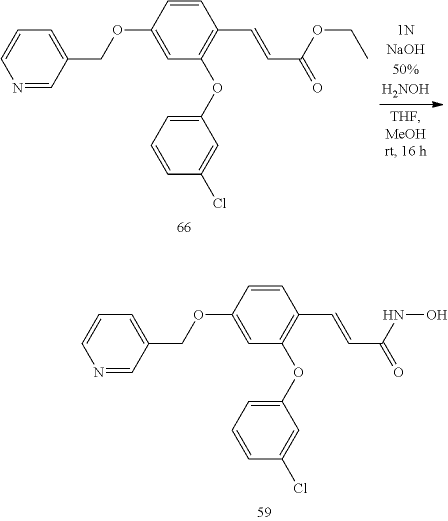 [SMILES: CCOC(=O)/C=C/c1ccc(OCc2cccnc2)cc1Oc1cccc(Cl)c1 ; O=C(/C=C/c1ccc(OCc2cccnc2)cc1Oc1cccc(Cl)c1)NO]

Step 5: To a stirred solution of 3-[2-(3-chloro-phenoxy)-4-(pyridin-3-ylmethoxy)-phenyl]-acrylic acid ethyl ester (66) (91 mg, 0.22 mmol) in THF (1.2 mL) and MeOH (1.2 mL) was added 50% solution of NH₂OH in water (0.73 mL, 11 mmol) and 1 N NaOH aq. (0.5 mL, 0.5 mmol) at rt. The resulting mixture was stirred at rt for 16 h. After the reaction mixture was concentrated under reduced pressure, the resulting aqueous suspension was diluted with DMSO (1.5 mL) and purified by HPLC to afford (E)-3-(2-(3-chlorophenoxy)-4-(pyridin-3-ylmethoxy)phenyl)-N-hydroxyacrylamide (59) (54 mg, 0.14 mmol, 62%) as a white solid. EM (calc): 396.09; ESI MS m/z 397.5 (M+H)⁺.

### Example 46

### Synthesis of (E)-3-(2-(3-chlorophenoxy)-4-(2-morpholinoethoxy)phenyl)-N-hydroxyacrylamide (67)

The title compound was synthesized as described in Example 45. EM (calc): 418.13; MS (M+1H)=419.5.

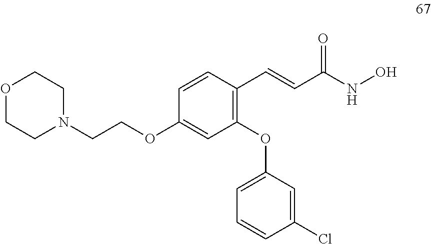 [SMILES: O=C(/C=C/c1ccc(OCCN2CCOCC2)cc1Oc1cccc(Cl)c1)NO]

### Example 47

### Synthesis of (E)-3-(2-(3-chlorophenoxy)-4-(2-(4-methylpiperazin-1-yl)ethoxy)phenyl)-N-hydroxyacrylamide (68)

The title compound was synthesized as described in Example 45. EM (calc): 431.16; MS (M+1H)=432.5.

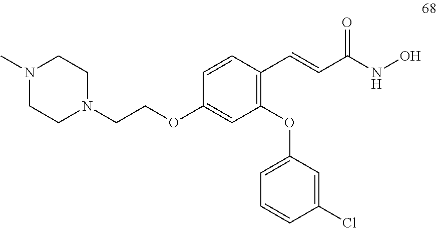 [SMILES: CN1CCN(CCOc2ccc(/C=C/C(=O)NO)c(Oc3cccc(Cl)c3)c2)CC1]

### Example 48

### Synthesis of (E)-3-(2-(3-chlorophenoxy)-4-(2-(dimethylamino)ethoxy)phenyl)-N-hydroxyacrylamide (69)

The title compound was synthesized as described in Example 45. EM (calc): 376.12; MS (M+1H)=377.5.

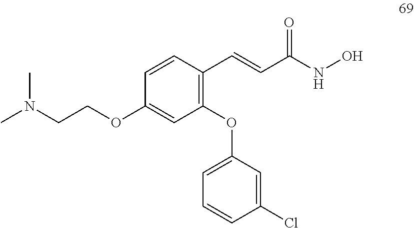 [SMILES: CN(C)CCOc1ccc(/C=C/C(=O)NO)c(Oc2cccc(Cl)c2)c1]

### Example 49

### Synthesis of (E)-3-(2-(3-chlorophenoxy)-4-(2-methoxyethoxy)phenyl)-N-hydroxyacrylamide (70)

The title compound was synthesized as described in Example 45. EM (calc): 363.09; MS (M+1H)=364.5.

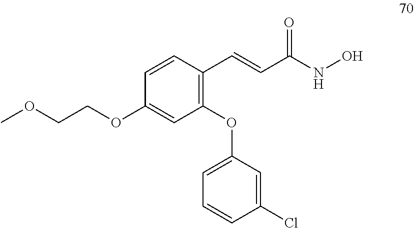 [SMILES: COCCOc1ccc(/C=C/C(=O)NO)c(Oc2cccc(Cl)c2)c1]

### Example 50

### Synthesis of (E)-3-(2-(3-fluorophenoxy)-4-(2-methoxyethoxy)phenyl)-N-hydroxyacrylamide (71)

The title compound was synthesized as described in Example 45. EM (calc): 347.12; MS (M+1H)=348.0.

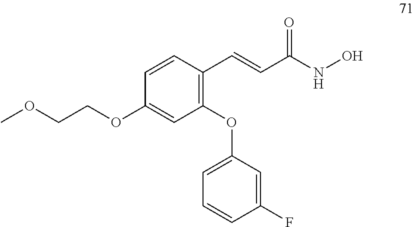 [SMILES: COCCOc1ccc(/C=C/C(=O)NO)c(Oc2cccc(F)c2)c1]

### Example 51

### Synthesis of (E)-3-(4-(2-acetamidoethoxy)-2-(3-chlorophenoxy)phenyl)-N-hydroxyacrylamide (72)

The title compound was synthesized as described in Example 45. EM (calc): 390.09; MS (M+1H)=391.5.

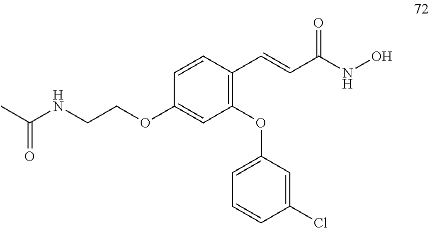 [SMILES: CC(=O)NCCOc1ccc(/C=C/C(=O)NO)c(Oc2cccc(Cl)c2)c1]

### Example 52

### Synthesis of (E)-3-(2-(3-fluorophenoxy)-4-(2-(dimethylamino)ethoxy)phenyl)-N-hydroxyacrylamide (73)

The title compound was synthesized as described in Example 45. EM (calc): 360.14; MS (M+1H)=361.0.

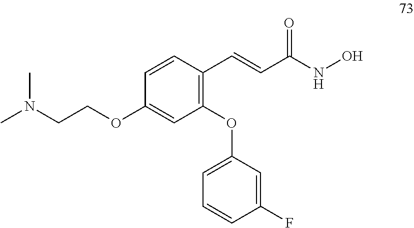 [SMILES: CN(C)CCOc1ccc(/C=C/C(=O)NO)c(Oc2cccc(F)c2)c1]

### Example 53

### Synthesis of (E)-3-(2-(3-fluorophenoxy)-4-(2-(4-methylpiperazin-1-yl)ethoxy)phenyl)-N-hydroxyacrylamide (74)

The title compound was synthesized as described in Example 45. EM (calc): 415.19; MS (M+1H)=416.5.

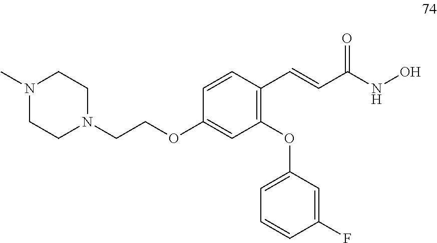 [SMILES: CN1CCN(CCOc2ccc(/C=C/C(=O)NO)c(Oc3cccc(F)c3)c2)CC1]

### Example 54

### Synthesis of (E)-3-(2-(4-fluorophenoxy)-4-(2-methoxyethoxy)phenyl)-N-hydroxyacrylamide (75)

The title compound was synthesized as described in Example 45. EM (calc): 347.12; MS (M+1H)=348.0.

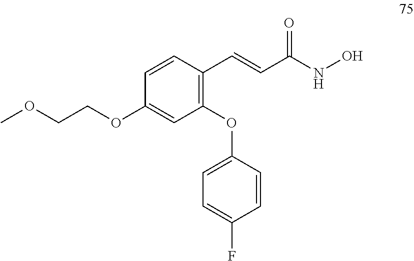 [SMILES: COCCOc1ccc(/C=C/C(=O)NO)c(Oc2ccc(F)cc2)c1]

### Example 55

### Synthesis of (E)-3-(2-(4-fluorophenoxy)-4-(pyridin-3-ylmethoxy)phenyl)-N-hydroxyacrylamide (76)

The title compound was synthesized as described in Example 45. EM (calc): 380.11; MS (M+1H)=381.5.

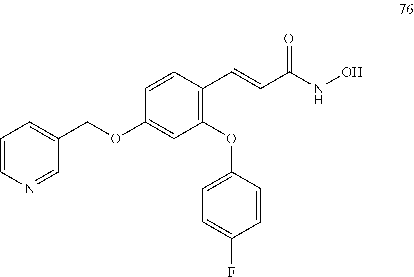 [SMILES: O=C(/C=C/c1ccc(OCc2cccnc2)cc1Oc1ccc(F)cc1)NO]

### Example 56

### Synthesis of (E)-3-(2-(4-fluorophenoxy)-4-(2-(dimethylamino)ethoxy)phenyl)-N-hydroxyacrylamide (77)

The title compound was synthesized as described in Example 45. EM (calc): 360.14; MS (M+1H)=361.5.

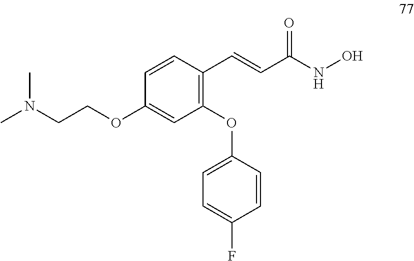 [SMILES: CN(C)CCOc1ccc(/C=C/C(=O)NO)c(Oc2ccc(F)cc2)c1]

### Example 57

### Synthesis of (E)-3-(4-(2-acetamidoethoxy)-2-(4-fluorophenoxy)phenyl)-N-hydroxyacrylamide (78)

The title compound was synthesized as described in Example 45. EM (calc): 374.12; MS (M+1H)=375.0.

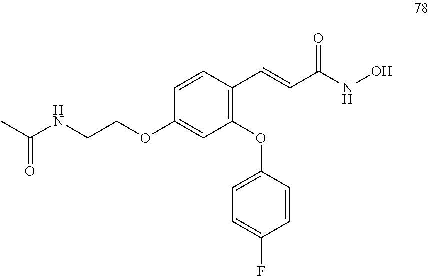 [SMILES: CC(=O)NCCOc1ccc(/C=C/C(=O)NO)c(Oc2ccc(F)cc2)c1]

### Example 58

### Synthesis of (E)-3-(2-(4-fluorophenoxy)-4-(2-(4-methylpiperazin-1-yl)ethoxy)phenyl)-N-hydroxyacrylamide (79)

The title compound was synthesized as described in Example 45. EM (calc): 415.19; MS (M+1H)=416.5.

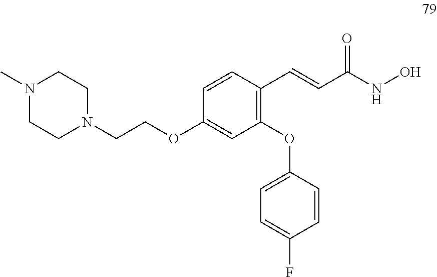 [SMILES: CN1CCN(CCOc2ccc(/C=C/C(=O)NO)c(Oc3ccc(F)cc3)c2)CC1]

### Example 59

### Synthesis of (E)-3-(2-(3,4-dichlorophenoxy)-4-(2-methoxyethoxy)phenyl)-N-hydroxyacrylamide (80)

The title compound was synthesized as described in Example 45. EM (calc): 397.04; MS (M+1H)=398.0.

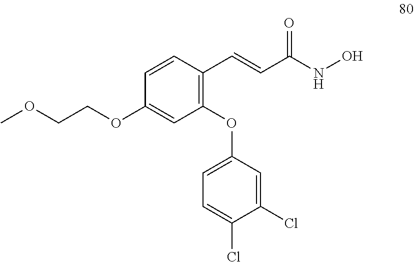 [SMILES: COCCOc1ccc(/C=C/C(=O)NO)c(Oc2ccc(Cl)c(Cl)c2)c1]

### Example 60

### Synthesis of (E)-3-(4-(2-acetamidoethoxy)-2-(3,4-dichlorophenoxy)phenyl)-N-hydroxyacrylamide (81)

The title compound was synthesized as described in Example 45. EM (calc): 424.05; MS (M+1H)=425.0.

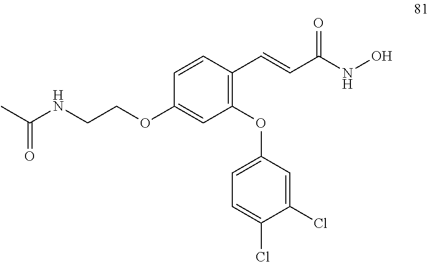 [SMILES: CC(=O)NCCOc1ccc(/C=C/C(=O)NO)c(Oc2ccc(Cl)c(Cl)c2)c1]

### Example 61

### Synthesis of (E)-3-(2-(3,4-dichlorophenoxy)-4-(2-morpholinoethoxy)phenyl)-N-hydroxyacrylamide (82)

The title compound was synthesized as described in Example 45. EM (calc): 452.09; MS (M+1H)=453.0.

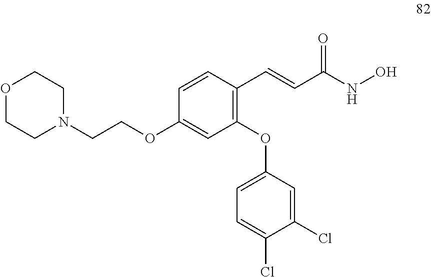 [SMILES: O=C(/C=C/c1ccc(OCCN2CCOCC2)cc1Oc1ccc(Cl)c(Cl)c1)NO]

### Example 62

### Synthesis of (E)-3-(2-(3,4-dichlorophenoxy)-4-(pyridin-3-ylmethoxy)phenyl)-N-hydroxyacrylamide (83)

The title compound was synthesized as described in Example 45. EM (calc): 430.04; MS (M+1H)=431.5.

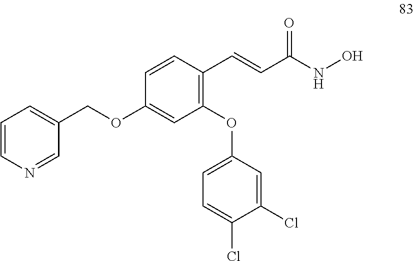 [SMILES: O=C(/C=C/c1ccc(OCc2cccnc2)cc1Oc1ccc(Cl)c(Cl)c1)NO]

### BIOLOGICAL EXAMPLES

### Cell Lines and Reagents

Cell lines are obtained from DSMZ (Braunschweig, Germany) or ATCC (Manassas, Va.). Cells are grown in RPMI 1640 with 10% fetal bovine serum in a 5% CO₂/air incubator at 37° C. Thapsigargin and BAPTA-AM are from Calbiochem (San Diego, Calif.). 3-((dimethylamino)methyl)-N-(2-(4-(hydroxycarbamoyl)phenoxy)ethyl)benzofuran-2-carboxamide is a broad-spectrum HDAC inhibitor which was synthesized as previously described. Other analogs with varying degrees of specificity towards the HDAC isoforms are synthesized as described herein.

### Example 63

### Histone Deacetylase Activity

HDAC activity is measured using a continuous trypsin-coupled assay that has been described in detail previously (US 20070281934; Schultz et. al., Biochemistry, 43 (34), 11083-11091, 2004; Kim et al. (2006), Methods Mol. Biol., 325:273-283). For inhibitor characterization, measurements are performed in a reaction volume of 100 □L using 96-well assay plates in a fluorescence plate reader. For each isozyme, the HDAC protein in reaction buffer (50 mM HEPES, 100 mM KCl, 0.001% Tween-20, 5% DMSO, pH 7.4, supplemented with bovine serum albumin at concentrations of 0-0.05%, is mixed with inhibitor at various concentrations and allowed to incubate for 15 minutes. Trypsin is added to a final concentration of 50 nM, and acetyl-Gly-Ala-(N-acetyl-Lys)-AMC is added to a final concentration of 25-100 μM to initiate the reaction. After a 30 minute lag time, the fluorescence is measured over a 30 minute time frame using an excitation wavelength of 355 nm and a detection wavelength of 460 nm. The increase in fluorescence with time is used as the measure of the reaction rate. Inhibition constants Kᵢ(app) were obtained using the program BatchKi (Biokin, Pullman, Wash.). The results are summarized in Table B below.

TABLE B
Comparison of HDAC IC₅₀ values of Representative
HDAC8-selective inhibitors

| Compound | | | HDAC6 IC₅₀ |
|---|---|---|---|
| No. | HDAC1 IC₅₀ (μM) | HDAC8 IC₅₀ (μM) | (μM) |
| 6 | C | A | C |
| 7 | C | A | B |
| 8 | C | A | B |
| 9 | C | A | C |
| 10 | C | A | B |
| 11 | C | A | B |
| 12 | C | A | C |
| 13 | C | A | C |
| 14 | C | A | C |
| 15 | C | A | B |
| 16 | C | A | C |
| 17 | D | A | D |
| 20 | C | A | C |
| 42 | D | A | D |
| 49 | D | A | C |
| 59 | C | A | B |
| 67 | C | A | B |
| 68 | C | A | B |
| 69 | B | A | B |
| 80 | D | A | C |

A = less than or equal to 0.1 μM
B = greater than 0.1 μM but less than or equal to 1 μM
C = greater than 1 μM but less than or equal to 10 μM
D = greater than 10 μM

### Example 64

### Cell Proliferation Assay

Tumor cell lines and human umbilical vein endothelial cells (HUVEC) are cultured for at least two doubling times, and growth is monitored at the end of compound exposure using an Alamar Blue™ (Biosource, Camarillo, Calif.) fluorometric cell proliferation assay as recommended by the manufacturer. Compounds are assayed in triplicate wells in 96-well plates. The concentration required to inhibit cell growth by 50% (GI₅₀) and 95% confidence intervals are estimated from nonlinear regression using a 4-parameter logistic equation. The effect of HDAC8 selective inhibitor compounds on cell proliferation in Jurkat cells is measured. Apoptosis is measured by Annexin-V flow cytommetry. Growth inhibiton iss measured by Alamar Blue assay. Growth Inhibition of Jurkat Cells measured by Alamar Blue assay is shown in Table C. Cells are treated with compound for 3 days.

TABLE C
Growth Inhibition of Jurkat Cells measured by Alamar Blue assay

| Compound | GI₅₀ (μM) |
|---|---|
| 6 | 1.43 |
| 7 | — |
| 8 | 6.1 |
| 9 | 2.00 |
| 10 | 2.25 |
| 11 | 1.98 |
| 12 | 1.80 |
| 13 | 2.5 |
| 14 | 3.58 |
| 15 | 3.37 |
| 16 | >20 |
| 17 | — |
| 20 | 9.70 |

### Example 65

### Western Blotting

Cells are washed with PBS and resuspended in triple-detergent lysis buffer [50 mM Tris-Cl (pH 8.0), 150 mM NaCl, 0.1% SDS, 0.5% deoxycholic acid, 1.0% NP-40, supplemented with 1 mM EDTA, 1 mM PMSF, 1 mM Na₃VO₄, 2 mM β-glycerophosphate and the COMPLETE protease inhibitor cocktail (Roche Molecular Biochemicals, Indianapolis, Ind.)] on ice for 10 minutes. After centrifugation, equal quantities of protein are resolved on SDS-polyacrylamide gels (Bio-Rad Laboratories, Hercules, Calif.). Gels are transferred to polyvinylidene difluoride membrane using a Semi-dry Transfer Cell (Bio-Rad Laboratories, Hercules, Calif.) and Western blotted, using an anti-Hsc70 antibody to control for loading and transfer. Bands are imaged and quantified in the linear range and normalized to Hsc70, using the Odyssey Infrared Imaging System (LICOR, Lincoln, Nebr.).

### Example 66

### Apoptosis Assays

Cytotoxicity is evaluated after 2 or 3 days of treatment with inhibitor alone and in combination with qVD, BAPTA-AM, thapsigargin and phospholipase C inhibitor using annexin-V staining Annexin-V binding is assayed with a FACSCalibur instrument (Becton-Dickinson, San Jose, Calif.) using reagents from BioVision (Mountain View, Calif.) per manufacturer's protocol.

### Example 67

### Caspase Activation Assays

Caspase enzyme activity iss measured in Jurkat cells using the Apotarget Caspase Colorimetric Protease Assay (BioSource International, Camarillo, Calif.) as per manufacturer's protocol following treatment with inhibitor.

### Example 68

### Intracellular Calcium Measurements

For the spectrofluorimetric measurements, cells (1×10⁶ cells/mL) are incubated for 1 h in Hanks' Balanced Salt Solution (HBSS; Invitrogen) containing 10% Fetal Bovine Serum and 5 □M Indol-AM (Invitrogen) at 37° C. in the dark. Cells are then harvested, centrifuged (200×g for 5 min) and washed three times with HBSS to remove extracellular Indol, and readjusted to 1×10⁶ cells/mL in HBSS. Fluorescence is monitored throughout each experiment at 37° C. with a fluorescent plate reader (Fluoroskan Ascent FL; Thermo Scientific). After a 5 min temperature equilibration period, samples are excited at 338 nm and emission is collected at 405 and 485 nm, corresponding to the Ca²⁺-bound and -free Indol fluorescence emitted respectively, at 6-sec intervals over a 1 minute period. Drug (or control) is then added, and acquisition is continued for 5 minutes. Maximal ratio values are determined by the addition of 100M ionomycin at the end of the measurements. Intracellular [Ca²⁺] changes are shown as changes in the ratio of Ca²⁺-bound and -free Indol.

### Example 69

### Pharmacokinetic Analysis of HDAC Inhibitor Compounds

This study, performed in male rats with test compounds is designed to provide preliminary information on their pharmacokinetics. The test compounds are administered in combination by oral gavage.

The specifications for rats used on this study are as follows:
Strain: CD® IGS rats (Sprague-Dawley derived)
Source: Charles River Laboratories
Surgical modification for oral dosing: One portal vein cannula and one jugular vein cannula
Body weight range at dosing 350 to 375 g

The rats are acclimatized to laboratory conditions for at least 24 hours before dosing. The evening before dosing, food is withheld from the rats and is returned immediately following the 3-hour blood collection time point. Water is provided ad libitum. The rats are housed individually in translucent polycarbonate cages.

Test compounds are prepared as 3.0 mg/ml solutions (1% MC/0.4% Cr EL in WFI).

Rats are administered a single dose of test compound in combination by oral gavage. Dose volumes are adjusted based on body weight data collected immediately prior to dosing.

The dose volume is 1 ml/kg and the nominal dosage is 3 mg/kg.

Blood samples are collected at 5 minutes, 20 minutes, 1 hour, 3 hours, 6 hours, 9 hours, and 24 hours post-dosing from orally dosed rats. The samples are collected into plasma separator Microtainer tubes with anticoagulant (lithium heparin). Plasma samples are prepared by centrifugation (5 min at 5000×g), and at least 100 nt, are transferred to storage tubes and frozen on dry ice. Samples are maintained at approximately −75 C until prepared for analysis.

Plasma samples are thawed and 75 uL aliquots are transferred to centrifuge tubes to which 10 μL aliquots of internal standard solution (0.5 μg/mL) are added. The samples are not diluted with blank plasma prior to further processing. Soluble proteins are precipitated by the addition of 300 μL of methanol, followed by centrifugation (20 min at 16,000×g). The samples are evaporated to dryness and reconstituted in 100 μL of water containing 0.2% formic acid and 10% methanol. All amples are loaded onto an autosampler maintained at 6° C. and evaluated for concentrations of test compound using LC-MS/MS. Plasma concentration data are evaluated using the computer program WinNonlin (Professional Edition, Pharsight Corporation, version 5.01). The analyses are performed using nominal sample times and a noncompartmental method with uniform weighting. Pharmacokinetic parameter estimates include terminal half-life, volume of distribution at steady state, and area under the concentration-time curve (AUC).

HDAC inhibitor 3-((dimethylamino)methyl)-N-(2-(4-(hydroxycarbamoyl)phenoxy)ethyl)benzofuran-2-carboxamide is added to the cassette to serve as a standard since the pharmacokinetics of this compound have been determined previously in rats.

### Example 70a

### Parenteral Composition

To prepare a parenteral pharmaceutical composition suitable for administration by injection, 100 mg of a water-soluble salt of a selective HDAC8 inhibitor compound described herein is dissolved in DMSO and then mixed with 10 mL of 0.9% sterile saline. The mixture is incorporated into a dosage unit form suitable for administration by injection.

In another embodiment, the following ingredients are mixed to form an injectable formulation.

| Ingredient | Amount | |
|---|---|---|
| Selective HDAC8 inhibitor compound | 1.2 | g |
| described herein | | |
| sodium acetate buffer solution (0.4M) | 2.0 | mL |
| HCl (1N) or NaOH (1M) | q.s. to suitable | |
| | pH | |
| water (distilled, sterile) | q.s. to 20 | mL |

All of the above ingredients, except water, are combined and heated to 60-70° C. with stirring. A sufficient quantity of water at 60° C. is then added with vigorous stirring to emulsify the ingredients, and water then added q.s. to 100 g.

### Example 70b

### Oral Composition

To prepare a pharmaceutical composition for oral delivery, 100 mg of a selective HDAC8 inhibitor compound described herein is mixed with 750 mg of starch. The mixture is incorporated into an oral dosage unit for, such as a hard gelatin capsule, which is suitable for oral administration.

In another embodiment, the following ingredients are mixed intimately and pressed into single scored tablets.

| | Quantity per tablet, |
|---|---|
| Ingredient | mg |
| selective HDAC8 inhibitor compound described | 400 |
| herein | |
| Cornstarch | 50 |
| croscarmellose sodium | 25 |
| Lactose | 120 |
| magnesium stearate | 5 |

In yet another embodiment, the following ingredients are mixed intimately and loaded into a hard-shell gelatin capsule.

| | Quantity per tablet, |
|---|---|
| Ingredient | mg |
| selective HDAC8 inhibitor compound described | 200 |
| herein | |
| lactose, spray-dried | 148 |
| magnesium stearate | 2 |

In yet another embodiment, the following ingredients are mixed to form a suspension for oral administration.

| Ingredient | Amount | |
|---|---|---|
| selective HDAC8 inhibitor compound described | 1.0 | g |
| herein | | |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution) | 12.85 | g |
| Veegum K (Vanderbilt Co.) | 1.0 | g |
| Flavoring | 0.035 | mL |
| Colorings | 0.5 | mg |
| distilled water | q.s. to 100 | mL |

### Example 70c

### Sublingual (Hard Lozenge) Composition

To prepare a pharmaceutical composition for buccal delivery, such as a hard lozenge, mix 100 mg of a selective HDAC8 inhibitor compound described herein with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 70d

### Inhalation Composition

To prepare a pharmaceutical composition for inhalation delivery, 20 mg of a selective HDAC8 inhibitor compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

### Example 70e

### Rectal Gel Composition

To prepare a pharmaceutical composition for rectal delivery, 100 mg of a selective HDAC8 inhibitor compound described herein is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 70f

### Suppository Formulation

A suppository of total weight 2.5 g is prepared by mixing a selective HDAC8 inhibitor compound described herein with Witepsol™ H-15 (triglycerides of saturated vegetable fatty acid; Riches-Nelson, Inc., New York), and has the following composition:

| | Quantity per |
|---|---|
| Ingredient | suppository (mg) |
| selective HDAC8 inhibitor compound described | 500 |
| herein | |
| Witepsol ® H-15 | balance |

### Example 70g

### Topical Gel Composition

To prepare a pharmaceutical topical gel composition, 100 mg of a selective HDAC8 inhibitor compound described herein is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topicl administration.

### Example 70h

### Ophthalmic Solution Composition

To prepare a pharmaceutical opthalmic solution composition, 100 mg of a selective HDAC8 inhibitor compound described herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

The examples and embodiments described herein are for illustrative purposes only and various modifications or changes are to be included within the spirit and purview of disclosure and scope of the appended claims.

What is claimed is:

1. A compound having a structure of Formula I: 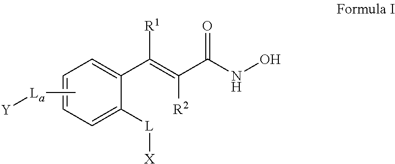
wherein:
R¹ and R² are each independently H, OH, halogen, or C₁-C₆alkyl;
L and Lₐ are each independently a bond, O, S, NR³, —NR¹⁰C(═O)—R¹¹, S(═O), S(═O)₂, NHS(═O)₂, —C₁-C₆alkylene-, —C₂-C₆alkenylene-, —C₂-C₆alkynylene-, —C₁-C₆heteroalkylene-, —C₁-C₆alkylene-O—, —C₁-C₃alkylene-O—C₁-C₃alkylene-, —C₁-C₆alkylene-NR³—, —C₁-C₃alkylene-NR³—C₁-C₃alkylene-, —C₁-C₆alkylene-C(═O)NR³—, —C₁-C₃alkylene-C(═O)NR³—C₁-C₃alkylene-, —C₁-C₆alkylene-NR³C(═O)—, —C₁-C₃ alkylene-NR³C(═O)—C₁-C₃alkylene-, —C₁-C₆alkylene-S—, —C₁-C₃alkylene-S—C₁-C₃ alkylene-, —C₁-C₆alkylene-S(═O)—, —C₁-C₃alkylene-S(═O)—C₁-C₃alkylene, —C₁-C₆alkylene-S(═O)₂—, —C₁-C₃alkylene-S(═O)₂—C₁-C₃alkylene, C(═O)—, or C(═O)—C₁-C₆alkylene;
X is a substituted or unsubstituted group selected from among aryl, heteroaryl, C₃-C₁₀cycloalkyl, and C₂-C₁₀heterocycloalkyl; where if X is substituted, then X is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
Y is H or a substituted or unsubstituted group selected from among C₁-C₆alkyl, —CO₂R¹⁰, —C(═O)R¹¹, —NR¹⁰C(═O)—R¹¹, —C(═O)N(R¹⁰)₂, aryl, heteroaryl, C₃-C₁₀cycloalkyl, and C₂-C₁₀heterocycloalkyl; where if Y is substituted, then Y is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is H, C₁-C₆alkyl, phenyl or benzyl;
or a pharmaceutically acceptable salt, pharmaceutically acceptable N-oxide, or pharmaceutically acceptable prodrug thereof.

2. A compound having the structure of Formula (IV), Formula (IVa), Formula (IVb), or Formula (IVc): 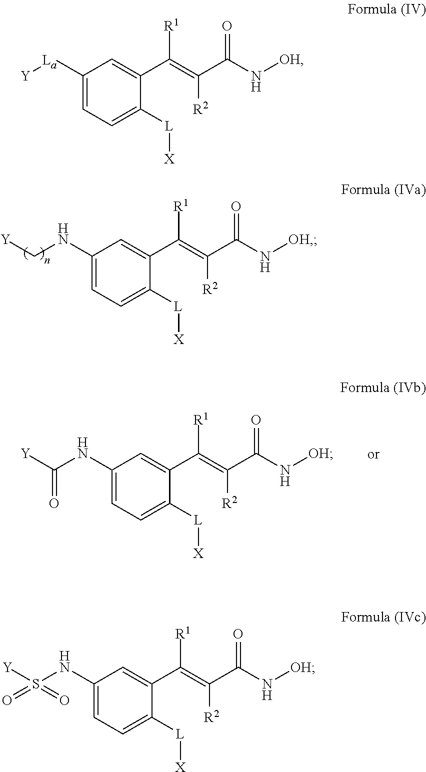
wherein
L and Lₐ are each independently a bond, O, S, NR³, —NR¹⁰C(═O)—R¹¹, S(═O), S(═O)₂, NHS(═O)₂, —C₁-C₆alkylene-, —C₂-C₆alkenylene-, —C₂-C₆alkynylene-, —C₁-C₆heteroalkylene-, —C₁-C₆alkylene-O—, —C₁-C₃alkylene-O—C₁-C₃alkylene-, —C₁-C₆alkylene-NR³—, —C₁-C₃alkylene-NR³—C₁-C₃alkylene-, —C₁-C₆alkylene-C(═O)NR³—, —C₁-C₃alkylene-C(═O)NR³—C₁-C₃alkylene-, —C₁-C₆alkylene-NR³C(═O)—, —C₁-C₃ alkylene-NR³C(═O)—C₁-C₃alkylene-, —C₁-C₆alkylene-S—, —C₁-C₃alkylene-S—C₁-C₃ alkylene-, —C₁-C₆alkylene-S(═O)—, —C₁-C₃alkylene-S(═O)—C₁-C₃alkylene, —C₁-C₆alkylene-S(═O)₂—, —C₁-C₃alkylene-S(═O)₂—C₁-C₃alkylene, —C(═O)—, or —C(═O)—C₁-C₆alkylene;
X is a substituted or unsubstituted group selected from among aryl, heteroaryl, C₃-C₁₀cycloalkyl, and C₂-C₁₀heterocycloalkyl; where if X is substituted, then X is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃ alkylaminoC₁-C₃alkoxy, C₂-C₈heterocycloalkylC₁-C₃alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)2, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹, N(R¹⁰)₂, —C₁-C₂alkylN(R¹⁰)₂, C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
Y is H or a substituted or unsubstituted group selected from among C₁-C₆alkyl, —CO₂R¹⁰, —C(═O)R¹¹, —NR¹⁰C(═O)—R¹¹, —C(═O)N(R¹⁰)₂, aryl, heteroaryl, C₃-C₁₀cycloalkyl, and C₂-C₁₀heterocycloalkyl; where if Y is substituted, then Y is substituted with 1, 2, 3, 4, or 5 groups selected from among halogen, C₁-C₆alkoxy, C₁-C₆fluoroalkoxy, aminoC₁-C₆alkoxy, C₁-C₃alkylaminoC₁-C₃alkoxy, hydroxyC₁-C₃alkylaminoC₁-C₃alkoxy, C₂-C₈heterocyclo alkylC₁-C₃ alkoxy, C₂-C₈heterocycloalkylC₁-C₂alkyl, —CN, —NO₂, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂, —OC(═O)N(R¹⁰)₂, —NR¹⁰C(═O)O—R¹¹, —OC(═O)O—R¹¹, —NHC(═O)NH—R¹¹, —OC(═O)—R¹¹, —N(R¹⁰)₂, —C₁-C₂alkylN(R¹⁰)₂. C₁-C₆alkyl, C₁-C₆fluoroalkyl, C₂-C₆alkenyl, C₂-C₆alkynyl, C₁-C₆heteroalkyl, C₃-C₈cycloalkyl, substituted or unsubstituted C₂-C₁₀heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is H, C₁-C₆alkyl, phenyl or benzyl;
R¹ and R² are each independently H; and n is an integer from 0 to 4.

3. The compound of claim 1 wherein R¹ and R² are each independently H; and L is O or S.

4. The compound of claim 1 wherein X is a substituted or unsubstituted phenyl.

5. (canceled)

6. The compound of claim 4 wherein
phenyl is substituted with at least one Cl, Br, I, or F; or
phenyl is substituted with at least two of Cl, Br, I, or F; or
phenyl is substituted with at least one C₁-C₆alkyl; or
phenyl is substituted with at least one C₁-C₆alkoxy.

7.-12. (canceled)

13. The compound of claim 1 wherein X is a substituted or unsubstituted heteroaryl.

14. The compound of claim 13 wherein heteroaryl is pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, 4-azaindolyl, 5-azaindolyl, 6-azaindolyl, 7-azaindolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, imidazo[1,2-a]pyridinyl, thiophenopyridinyl, and furopyridinyl.

15. The compound of claim 14 wherein heteroaryl is pyridinyl.

16. The compound of claim 1 wherein X is a substituted or unsubstituted C₃-C₁₀cycloalkyl.

17. (canceled)

18. The compound of claim 1 wherein X is a substituted or unsubstituted C₂-C₁₀heterocycloalkyl.

19. The compound of claim 18 wherein C₂-C₁₀heterocycloalkyl is quinolizinyl, dioxinyl, piperidinyl, morpholinyl, thiomorpholinyl, thiazinyl, tetrahydropyridinyl, piperazinyl, oxazinanonyl, dihydropyrrolyl, dihydroimidazolyl, tetrahydrofuranyl, tetrahydropyranyl, dihydrooxazolyl, oxiranyl, pyrrolidinyl, pyrazolidinyl, dihydrothienyl, imidazolidinonyl, pyrrolidinonyl, dihydrofuranonyl, dioxolanonyl, thiazolidinyl, piperidinonyl, indolinyl, tetrahydroquinolinyl, tetrahydroisoquinolinyl, and tetrahydrothienyl.

20. The compound of claim 19 wherein C₂-C₁₀heterocycloalkyl is piperidinyl.

21. The compound of claim 20 wherein piperidinyl is substituted with —CO₂R¹⁰, —C(═O)R¹¹ or —C(═O)N(R¹⁰)₂.

22.-30. (canceled)

31. A compound selected from (E)-N-hydroxy-3-(2-(3-methoxyphenoxy)phenyl)acrylamide; (E)-3-(2-(3-fluorophenoxy)phenyl)-N-hydroxyacrylamide; (E)-N-hydroxy-3-(2-(pyridin-3-yloxy)phenyl)acrylamide; (E)-N-hydroxy-3-(2-(pyridin-4-yloxy)phenyl)acrylamide; (E)-N-hydroxy-3-(2-(4-methoxyphenoxy)phenyl)acrylamide; (E)-3-(2-(3-chlorophenoxy)phenyl)-N-hydroxyacrylamide; (E)-3-(2-(3,4-dichlorophenoxy)phenyl)-N-hydroxyacrylamide; (E)-N-hydroxy-3-(2-(m-tolyloxy)phenyl)acrylamide; (E)-N-hydroxy-3-(2-phenoxyphenyl)acrylamide; (E)-N-hydroxy-3-(2-(p-tolyloxy)phenyl)acrylamide; (E)-3-(2-(4-chlorophenoxy)phenyl)-N-hydroxyacrylamide; (E)-3-(2-(4-fluorophenoxy)phenyl)-N-hydroxyacrylamide; (S,E)-3-(2-(1-benzoylpiperidin-3-yloxy)phenyl)-N-hydroxyacrylamide; (S,E)-3-(2-(1-acetylpiperidin-3-yloxy)phenyl)-N-hydroxyacrylamide; (S,E)-N-hydroxy-3-(2-(1-isobutyrylpiperidin-3-yloxy)phenyl)acrylamide; (E)-3-(2-(1-(furan-2-carbonyl)piperidin-4-yloxy)phenyl)-N-hydroxyacrylamide; (E)-3-(2-(1-acetylpiperidin-4-yloxy)phenyl)-N-hydroxyacrylamide; (E)-N-hydroxy-3-(2-(1-isobutyrylpiperidin-4-yloxy)phenyl)acrylamide; (E)-3-(2-(1-benzoylpiperidin-4-yloxy)phenyl)-N-hydroxyacrylamide; (E)-N-hydroxy-3-(2-(1-nicotinoylpiperidin-4-yloxy)phenyl)acrylamide; (R,E)-3-(2-(1-acetylpiperidin-3-yloxy)phenyl)-N-hydroxyacrylamide; (S,E)-3-(2-(1-(furan-2-carbonyl)piperidin-3-yloxy)phenyl)-N-hydroxyacrylamide; (R,E)-3-(2-(1-(furan-2-carbonyl)piperidin-3-yloxy)phenyl)-N-hydroxyacrylamide; (R,E)-N-hydroxy-3-(2-(1-isobutyrylpiperidin-3-yloxy)phenyl)acrylamide; (R,E)-3-(2-(1-benzoylpiperidin-3-yloxy)phenyl)-N-hydroxyacrylamide; (R,E)-N-hydroxy-3-(2-(1-nicotinoylpiperidin-3-yloxy)phenyl)acrylamide; (S,E)-N-hydroxy-3-(2-(1-nicotinoylpiperidin-3-yloxy)phenyl)acrylamide; (E)-N-(4-(4-fluorophenoxy)-3-(3-(hydroxyamino)-3-oxoprop-1-enyl)phenyl)nicotinamide; (E)-3-(5-acetamido-2-(4-fluorophenoxy)phenyl)-N-hydroxyacrylamide; (E)-3-(5-acetamido-2-(3-chlorophenoxy)phenyl)-N-hydroxyacrylamide; (E)-N-(4-(3-chlorophenoxy)-3-(3-(hydroxyamino)-3-oxoprop-1-enyl)phenyl)nicotinamide; (E)-N-(4-(3-fluorophenoxy)-3-(3-(hydroxyamino)-3-oxoprop-1-enyl)phenyl)nicotinamide; (E)-N-(4-(3,4-dichlorophenoxy)-3-(3-(hydroxyamino)-3-oxoprop-1-enyl)phenyl)nicotinamide; (E)-3-(5-acetamido-2-(3-fluorophenoxy)phenyl)-N-hydroxyacrylamide; (E)-3-(2-(4-fluorophenoxy)-5-(methylsulfonamido)phenyl)-N-hydroxyacrylamide; (E)-3-(2-(3-chlorophenoxy)-5-(methylsulfonamido)phenyl)-N-hydroxyacrylamide; (E)-3-(2-(4-fluorophenoxy)-5-(pyridin-3-ylmethylamino)phenyl)-N-hydroxyacrylamide; (E)-3-(2-(3-chlorophenoxy)-5-(pyridin-3-ylmethylamino)phenyl)-N-hydroxyacrylamide; (E)-3-(2-(3-chlorophenoxy)-5-(pyridin-2-ylmethylamino)phenyl)-N-hydroxyacrylamide; (E)-3-(2-(4-fluorophenoxy)-5-(pyridin-2-ylmethylamino)phenyl)-N-hydroxyacrylamide; (E)-3-(2-(3-fluorophenoxy)-5-(pyridin-3-ylmethylamino)phenyl)-N-hydroxyacrylamide; (E)-3-(2-(3,4-dichlorophenoxy)-5-(pyridin-3-ylmethylamino)phenyl)-N-hydroxyacrylamide; (E)-3-(2-(3-fluorophenoxy)-5-(pyridin-2-ylmethylamino)phenyl)-N-hydroxyacrylamide; (E)-3-(2-(3,4-dichlorophenoxy)-5-(pyridin-2-ylmethylamino)phenyl)-N-hydroxyacrylamide; (E)-3-(2-(3-chlorophenoxy)-4-(pyridin-3-ylmethoxy)phenyl)-N-hydroxyacrylamide; (E)-3-(2-(3-chlorophenoxy)-4-(2-morpholinoethoxy)phenyl)-N-hydroxyacrylamide; (E)-3-(2-(3-chlorophenoxy)-4-(2-(4-methylpiperazin-1-yl)ethoxy)phenyl)-N-hydroxyacrylamide; (E)-3-(2-(3-chlorophenoxy)-4-(2-(dimethylamino)ethoxy)phenyl)-N-hydroxyacrylamide; (E)-3-(2-(3-chlorophenoxy)-4-(2-methoxyethoxy)phenyl)-N-hydroxyacrylamide; (E)-3-(2-(3-fluorophenoxy)-4-(2-methoxyethoxy)phenyl)-N-hydroxyacrylamide; (E)-3-(4-(2-acetamidoethoxy)-2-(3-chlorophenoxy)phenyl)-N-hydroxyacrylamide; (E)-3-(2-(3-fluorophenoxy)-4-(2-(dimethylamino)ethoxy)phenyl)-N-hydroxyacrylamide; (E)-3-(2-(3-fluorophenoxy)-4-(2-(4-methylpiperazin-1-yl)ethoxy)phenyl)-N-hydroxyacrylamide; (E)-3-(2-(4-fluorophenoxy)-4-(2-methoxyethoxy)phenyl)-N-hydroxyacrylamide; (E)-3-(2-(4-fluorophenoxy)-4-(pyridin-3-ylmethoxy)phenyl)-N-hydroxyacrylamide; (E)-3-(2-(4-fluorophenoxy)-4-(2-(dimethylamino)ethoxy)phenyl)-N-hydroxyacrylamide; (E)-3-(4-(2-acetamidoethoxy)-2-(4-fluorophenoxy)phenyl)-N-hydroxyacrylamide; (E)-3-(2-(4-fluorophenoxy)-4-(2-(4-methylpiperazin-1-yl)ethoxy)phenyl)-N-hydroxyacrylamide; (E)-3-(2-(3,4-dichlorophenoxy)-4-(2-methoxyethoxy)phenyl)-N-hydroxyacrylamide; (E)-3-(4-(2-acetamidoethoxy)-2-(3,4-dichlorophenoxy)phenyl)-N-hydroxyacrylamide; (E)-3-(2-(3,4-dichlorophenoxy)-4-(2-morpholinoethoxy)phenyl)-N-hydroxyacrylamide; and (E)-3-(2-(3,4-dichlorophenoxy)-4-(pyridin-3-ylmethoxy)phenyl)-N-hydroxyacrylamide; an active metabolite, pharmaceutically acceptable solvate, pharmaceutically acceptable salt, pharmaceutically acceptable N-oxide, or pharmaceutically acceptable prodrug thereof.

32. A pharmaceutical composition comprising (a) a compound of claim 1 or an active metabolite, pharmaceutically acceptable solvate, pharmaceutically acceptable salt, pharmaceutically acceptable N-oxide, or pharmaceutically acceptable prodrug thereof, and (b) a pharmaceutically acceptable diluent, excipient, or carrier.

33.-34. (canceled)

35. A method of treating T-cell lymphoma or leukemia in a mammal in need thereof, comprising administering to the mammal a pharmaceutical composition containing a therapeutically effective amount of a compound of claim 1.

36. The method of claim 35, further comprising administering to the mammal a second therapeutic agent, selected from among abarelix; aldesleukin; Aldesleukin; Alemtuzumab; alitretinoin; allopurinol; altretamine; amifostine; anastrozole; arsenic trioxide; asparaginase; azacitidine; bevacizumab; bexarotene; bleomycin; bortezomib; busulfan; busulfan; calusterone; capecitabine; carboplatin; carmustine; carmustine; celecoxib; cetuximab; chlorambucil; cisplatin; cladribine; clofarabine; cyclophosphamide; cytarabine; cytarabine liposomal; dacarbazine; dactinomycin; Darbepoetin alfa; dasatinib; daunorubicin liposomal; daunorubicin; daunorubicin; decitabine; denileukin; dexrazoxane; docetaxel; doxorubicin; doxorubicin liposomal; dromostanolone propionate; epirubicin; Epirubicin; Epoetin alfa; erlotinib; estramustine; etoposide phosphate; etoposide; exemestane; Filgrastim; floxuridine; fludarabine; fluorouracil; fulvestrant; gefitinib; gemcitabine; gemtuzumab ozogamicin; goserelin acetate; histrelin acetate; hydroxyurea; Ibritumomab Tiuxetan; idarubicin; ifosfamide; imatinib mesylate; interferon alfa 2a; Interferon alfa-2b; irinotecan; lenalidomide; letrozole; leucovorin; Leuprolide Acetate; levamisole; lomustine; meclorethamine, nitrogen mustard; megestrol acetate; melphalan; mercaptopurine; methotrexate; methoxsalen; mitomycin C; mitomycin C; mitotane; mitoxantrone; nandrolone phenpropionate; nelarabine; Nofetumomab; Oprelvekin; oxaliplatin; paclitaxel; paclitaxel; paclitaxel protein-bound particles; palifermin; pamidronate; panitumumab; pegademase; pegaspargase; Pegfilgrastim; pemetrexed disodium; pentostatin; pipobroman; plicamycin, mithramycin; porfimer sodium; procarbazine; quinacrine; Rasburicase; rituximab; sargramostim; Sargramostim; sorafenib; streptozocin; sunitinib maleate; tamoxifen; temozolomide; teniposide; testolactone; thalidomide; thioguanine; thiotepa; topotecan; toremifene; tositumomab; tositumomab/1-131 tositumomab; trastuzumab; tretinoin; Uracil Mustard; valrubicin; vinblastine; vincristine; vinorelbine; vorinostat; zoledronate; and zoledronic acid.

37.-38. (canceled)

39. A method of treating a disease or condition mediated by interleukin-1 beta (IL-1b) or IL-18 in a mammal, comprising administering to the mammal a therapeutically effective amount of a compound of claim 1, or a pharmaceutically acceptable salt, pharmaceutically acceptable N-oxide, pharmaceutically active metabolite, pharmaceutically acceptable prodrug, or pharmaceutically acceptable solvate thereof.

40. The method of claim 39, wherein the disease or condition is selected from among osteoarthritis, rheumatoid arthritis, septic arthritis, gout, pseudogout, juvenile arthritis, Still's disease, Ankylosing spondylitis, systemic lupus erythematosus (SLE), Henoch-Schnlein purpura, psoriatic arthritis, reactive arthritis (Reiter's syndrome), hemochromatosis, hepatitis, Wegener's granulomatosis, Familial Mediterranean fever (FMF), HIDS (hyperimmunoglobulinemia D and periodic fever syndrome), TRAPS (TNF-alpha receptor associated periodic fever syndrome), inflammatory bowel disease, Crohn's Disease, ulcerative colitis, recurrent fever, anemia, leukocytosis, asthma, chronic obstructive pulmonary disease, and myalgia.

41. The method of claim 40, further comprising administering to the mammal a second therapeutic agent, selected from among tacrolimus, cyclosporin, rapamicin, methotrexate, cyclophosphamide, azathioprine, mercaptopurine, mycophenolate, or FTY720, prednisone, cortisone acetate, prednisolone, methylprednisolone, dexamethasone, betamethasone, triamcinolone, beclometasone, fludrocortisone acetate, deoxycorticosterone acetate, aldosterone, aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502, JTE-522, L-745,337 and NS398, leflunomide, gold thioglucose, gold thiomalate, aurofin, sulfasalazine, hydroxychloroquinine, minocycline, infliximab, etanercept, adalimumab, abatacept, anakinra, interferon-β, interferon-γ, interleukin-2, allergy vaccines, antihistamines, antileukotrienes, beta-agonists, theophylline, and anticholinergics.

42.-43. (canceled)
